(19) United States
(12) Patent Application Publication
(10) Pub. No.: US 2026/0053925 A1
(43) Pub. Date: Feb. 26, 2026

(54) DOSING REGIMENS FOR CD19-DIRECTED CANCER IMMUNOTHERAPY

(71) Applicant: NKARTA, INC., South San Francisco, CA (US)

(72) Inventors: James Barnaby Trager, South San Francisco, CA (US); David Richard Shook, South San Francisco, CA (US); Kanya Lakshmi Rajangam, South San Francisco, CA (US); Carmel T. Chan, South San Francisco, CA (US); Mira Tohme, South San Francisco, CA (US); Joanne Tan, South San Francisco, CA (US)

(21) Appl. No.: 18/877,116

(22) PCT Filed: Jun. 29, 2023
(86) PCT No.: PCT/US2023/069403

§ 371 Date: Dec. 19, 2024

Related U.S. Application Data
(60) Provisional application No. 63/367,582, filed on Jul. 1, 2022.
(60) Provisional application No. 63/380,432, filed on Oct. 21, 2022.
(60) Provisional application No. 63/382,863, filed on Nov. 8, 2022.
(60) Provisional application No. 63/499,440, filed on May 1, 2023.

(51) Int. Cl. A61K 40/15; A61K 39/395; A61K 40/31; A61K 40/42; A61P 35/00

(52) U.S. Cl. CPC A61K 40/15; A61K 39/39558; A61K 40/31; A61K 40/4211; A61P 35/00; A61K 2239/21; A61K 2239/22; A61K 2239/38

(57) ABSTRACT

Several embodiments of the methods and compositions disclosed herein relate to immune cells that are engineered to express cytotoxic chimeric receptors and various dosing regimens for administering such cells. In several embodiments, the immune cells express a chimeric receptor that targets CD19 on tumor cells. In several embodiments, the cancer is a blood cancer, for example, B cell malignancy. In several embodiments, the immune cells engineered to express a CD 19-directed CAR are administered in combination (prior to, after or concurrently) with an antibody therapy, such as an anti-CD20 directed antibody.

### FIELD

Several embodiments disclosed herein relate to methods and compositions comprising genetically engineered cells for cancer immunotherapy. In several embodiments, the present disclosure relates to cells engineered to express chimeric antigen receptors directed to a particular tumor marker, such as CD19, and administration of such cells in accordance with certain dosing regimens to achieve successful cancer immunotherapy.

### BACKGROUND

As further knowledge is gained about various cancers and what characteristics a cancerous cell has that can be used to specifically distinguish that cell from a healthy cell, therapeutics are under development that leverage the distinct features of a cancerous cell. Immunotherapies that employ engineered immune cells are one approach to treating cancers.

### INCORPORATION BY REFERENCE OF MATERIAL IN SEQUENCE LISTING FILE

This application incorporates by reference the Sequence Listing contained in the following XML text file being submitted concurrently herewith: File name: NKT.088WO_ST26.xml; created on Jun. 29, 2023 and is 60,180 bytes in size.

### SUMMARY

Immunotherapy presents a new technological advancement in the treatment of disease, wherein immune cells are engineered to express certain targeting and/or effector molecules that specifically identify and react to diseased or damaged cells. This represents a promising advance due, at least in part, to the potential for specifically targeting diseased or damaged cells, as opposed to more traditional approaches, such as chemotherapy, where all cells are impacted, and the desired outcome is that sufficient healthy cells survive to allow the patient to live. One immunotherapy approach is the recombinant expression of chimeric antigen receptors, also referred to as CARs or chimeric receptors, in immune cells to achieve the targeted recognition and destruction of aberrant cells of interest.

In several embodiments, there is provided herein a population of genetically engineered natural killer (NK) cells for cancer immunotherapy, comprising a plurality of NK cells that have been expanded in culture, wherein the plurality of NK cells are engineered to express a chimeric antigen receptor comprising a CD19-targeting extracellular domain, a transmembrane domain, and a cytotoxic signaling complex. In several embodiments, the CD19-targeting extracellular domain binds to CD19. In several embodiments, the CD19-targeting extracellular domain binds to human CD19.

In several embodiments, there is provided a dosing regimen for cancer immunotherapy, comprising at least a first dosing cycle, wherein the first dosing cycle comprises a first dose of genetically engineered natural killer (NK) cells, a second dose of genetically engineered NK cells, and a third dose of genetically engineered NK cells, wherein the first dose is administered to a subject in need of cancer immunotherapy at a first time point, wherein the second dose is administered to the subject between 5-10 days after the first time point, and wherein the third dose is administered to the subject between 5-10 days after the second dose; wherein each of the first, second and third doses comprises about 1.5×10⁹ NK cells, wherein at least a portion of the genetically engineered NK cells is engineered to express a chimeric antigen receptor (CAR) that is directed against a CD19 tumor marker, wherein the first dosing cycle is initiated after the subject has undergone a lymphodepletion process in order to reduce native immune cell numbers. In some embodiments, the first dosing cycle is followed by one or more additional dosing cycle.

In several embodiments, there is provided a dosing regimen for cancer immunotherapy, comprising at least a first dosing cycle, wherein the first dosing cycle comprises a first dose of genetically engineered natural killer (NK) cells, a second dose of genetically engineered NK cells, and a third dose of genetically engineered NK cells, wherein the first dose is administered to a subject in need of cancer immunotherapy at a first time point, wherein the second dose is administered to the subject between 5-10 days after the first time point, and wherein the third dose is administered to the subject between 5-10 days after the second dose; wherein each of the first, second and third doses comprises about 1.5×10⁹ NK cells, wherein at least a portion of the genetically engineered NK cells is engineered to express a chimeric antigen receptor (CAR) that is directed against a CD19 tumor marker, wherein the first dosing cycle is initiated after the subject has undergone a lymphodepletion process in order to reduce native immune cell numbers, and wherein the first dosing cycle is optionally followed by one or more additional dosing cycle. In several embodiment, subjects exhibiting at least a partial response will receive at least one additional dosing cycle. Dosing cycles may continue, depending on the embodiment as long as the subject is exhibiting an anti-tumor response and tolerating the engineering NK cells. In several embodiments, a subject will not receive an additional dosing cycle if they are not responding (e.g., no tumor response) and/or if the therapy is not tolerated. However, as discussed herein, in several embodiments the disclosed dosing regimens have limited, or no, adverse impacts or toxicities. In several embodiments, a determination about receiving/administering an additional dosing cycle is made at an evaluation 30 days after the inception of a dosing cycle (whether that be the first dosing cycle, or a subsequent cycle). In several embodiments, not more than 5 additional cycles are given to a subject.

In several embodiments, the dosing regimens provided for herein further comprise administration of an additional therapeutic agent that targets a CD20 tumor marker.

In some such embodiments, at least about 1.0×10⁹ NK cells are administered at each dose in such a combination therapy. In some such embodiments, about 1.0×10⁹ NK cells are administered at each dose in such a combination therapy. In several embodiments, however, at least about 1.5×10⁹ NK cells are used in a combination therapy with an agent that targets CD20. In several embodiments, however, about 1.5×10⁹ NK cells are used in a combination therapy with an agent that targets CD20. Depending on the embodiment, the additional therapeutic agent is an antibody or a biosimilar. In several embodiments, the additional therapeutic agent is administered in an amount between about 150 mg/m² and about 500 mg/m². In several embodiments, the additional therapeutic agent is administered in an amount between about 200 mg/m² and about 400 mg/m². In several embodiments, the additional therapeutic agent is administered in an amount between about 350 mg/m² and about 425 mg/m². In several embodiments, the additional therapeutic agent is administered in an amount of about 375 mg/m². In several embodiments, the additional therapeutic agent is administered at least one time and at least 2 days prior to administration the first dose of a dosing cycle. In several embodiments, the additional therapeutic agent is administered one time and wherein the additional therapeutic is administered 3 days prior to administration the first dose of the dosing cycle. In several embodiments, the additional therapeutic agent is a biosimilar selected from rituximab-abbs, rituximab-arrx, and/or rituximab-pvvr. In several embodiments, the additional therapeutic agent is an anti-CD20 monoclonal antibody. In several embodiments, the anti-CD20 monoclonal antibody is selected from rituximab, ocrelizumab, ofatumumab, obinutuzumab, ibritumomab, ibritumomab or combinations thereof. In several embodiments, the anti-CD20 antibody is rituximab. In some embodiments, the anti-CD20 antibody is obinutuzumab.

Also provided for herein is a dosing regimen for cancer immunotherapy, comprising at least a first dosing cycle, wherein the first dosing cycle comprises a first dose of genetically engineered natural killer (NK) cells, a second dose of genetically engineered NK cells, and a third dose of genetically engineered NK cells, wherein the first dose is administered to a subject in need of cancer immunotherapy at a first time point, wherein the second dose is administered to the subject between 5-10 days after the first time point, wherein the third dose is administered to the subject between 5-10 days after the second dose; wherein each of the first, second and third doses comprises about 1.5×10⁹ NK cells, wherein at least a portion of the engineered NK cells is engineered to express a chimeric antigen receptor (CAR) that is directed against a CD19 tumor marker, wherein the first dosing cycle is initiated after the subject has undergone a lymphodepletion process comprising at least two doses of cyclophosphamide and fludarabine, wherein an anti-CD20 antibody is administered during the lymphodepletion process. In some embodiments, the first dosing cycle is followed by one or more additional dosing cycle.

Also provided for herein is a dosing regimen for cancer immunotherapy, comprising at least a first dosing cycle, wherein the first dosing cycle comprises a first dose of genetically engineered natural killer (NK) cells, a second dose of genetically engineered NK cells, and a third dose of genetically engineered NK cells, wherein the first dose is administered to a subject in need of cancer immunotherapy at a first time point, wherein the second dose is administered to the subject between 5-10 days after the first time point, wherein the third dose is administered to the subject between 5-10 days after the second dose; wherein each of the first, second and third doses comprises about 1.5×10⁹ NK cells, wherein at least a portion of the engineered NK cells is engineered to express a chimeric antigen receptor (CAR) that is directed against a CD19 tumor marker, wherein the first dosing cycle is initiated after the subject has undergone a lymphodepletion process comprising at least two doses of cyclophosphamide and fludarabine, wherein an anti-CD20 antibody is administered during the lymphodepletion process, and wherein the first dosing cycle is optionally followed by one or more additional dosing cycle.

In several embodiments, the NK cells are CAR-expressing NK cells. In several embodiments, each of the first, second and third doses comprises about 1.5×10⁹ CAR-expressing NK cells.

In some embodiments, among subjects treated according to the dosing regimen, the overall response rate (ORR) is at least about 50%, at least about 60%, at least about 70%, or at least about 80%. In some embodiments, among subjects treated according to the dosing regimen, the ORR is at least about 50%. In some embodiments, among subjects treated according to the dosing regimen, the ORR is at least about 60%. In some embodiments, among subjects treated according to the dosing regimen, the ORR is at least about 70%. In some embodiments, among subjects treated according to the dosing regimen, the ORR is at least about 80%.

In some embodiments, at least about 50%, at least about 60%, at least about 70%, or at least about 80% of subjects treated according to the dosing regimen exhibit a complete response (CR). In some embodiments, at least about 50% of subjects treated according to the dosing regimen exhibit a CR. In some embodiments, at least about 60% of subjects treated according to the dosing regimen exhibit a CR. In some embodiments, at least about 70% of subjects treated according to the dosing regimen exhibit a CR. In some embodiments, at least about 80% of subjects treated according to the dosing regimen exhibit a CR.

In some embodiments, if the subject exhibits a clinical response following the first dosing cycle, the dosing regimen comprises an additional dosing cycle. In some embodiments, if the subject exhibits a complete response (CR) following the first dosing cycle, the dosing regimen comprises an additional dosing cycle. In some embodiments, if the subject exhibits a clinical response following a dosing cycle and subsequently exhibits disease progression, the dosing regimen comprises an additional dosing cycle. In some embodiments, the dosing regimen comprises between one dosing cycle and five dosing cycles. In some embodiments, the dosing regimen consists of between one dosing cycle and five dosing cycles. In some embodiments, the dosing regimen consists of between one dosing cycle and five dosing cycles. In some embodiments, the dosing regimen consists of one dosing cycle. In some embodiments, the dosing regimen consists of two dosing cycles. In some embodiments, the dosing regimen consists of three dosing cycles. In some embodiments, the dosing regimen consists of four dosing cycles. In some embodiments, the dosing regimen consists of five dosing cycles. In some embodiments, the subject underdoes a lymphodepletion process prior to each dosing cycle.

In some embodiments, cells of the cancer do not express CD58 or express a mutated form of CD58. In some embodiments, cells of the cancer do not express CD58. In some embodiments, cells of the cancer express a mutated form of CD58. In some embodiments, prior to administration of the first dosing cycle to the subject, cells of the cancer are determined not to express CD58 or to express a mutated form of CD58. In some embodiments, prior to administration of the first dosing cycle to the subject, the subject has been selected for treatment with the dosing regimen based on cells of the cancer exhibiting a loss or mutation of CD58. In some embodiments, the mutation of CD58 comprises a loss of function mutation.

In some embodiments, one dose of each dosing cycle is administered to the subject on an outpatient basis. In some embodiments, each dose of each dosing cycle is administered to the subject on an outpatient basis.

Also provided herein is a method for the treatment of cancer, comprising administering to a subject with a cancer genetically engineered natural killer (NK) cells expressing a chimeric antigen receptor (CAR) that is directed against an antigen associated with or expressed by cells of the cancer, wherein cells of the cancer do not express CD58 or expresses a mutated form of CD58, and wherein the subject is relapsed and/or refractory to genetically engineered T cells expressing a CAR that is directed against the antigen.

Also provided herein is a method for the treatment of cancer, comprising administering genetically engineered natural killer (NK) cells expressing a chimeric antigen receptor (CAR) that is directed against CD19 to a subject with a cancer that does not express CD58 or expresses a mutated form of CD58.

Also provided herein is a method for the treatment of cancer, comprising: (a) identifying a subject having a cancer that does not express CD58 or expresses a mutated form of CD58; (b) selecting the identified subject for treatment with genetically engineered natural killer (NK) cells expressing a chimeric antigen receptor (CAR) that is directed against CD19; and (c) administering the genetically engineered NK cells to the selected subject.

In some embodiments, prior to identifying the subject, the method further comprises determining whether the cancer expresses CD58 or expresses a mutated form of CD58. In some embodiments, the subject was previously treated with genetically engineered T cells expressing a CAR that is directed against CD19 for the cancer. In some embodiments, the subject is relapsed and/or refractory to the genetically engineered T cells. In some embodiments, the administering comprises administering to the selected subject at least a first dosing cycle, wherein the first dosing cycle comprises a first dose of the genetically engineered NK cells, a second dose of the genetically engineered NK cells, and a third dose of the genetically engineered NK cells, wherein the first dose is administered to the subject at a first time point, wherein the second dose is administered to the subject between 5-10 days after the first time point, wherein the third dose is administered to the subject between 5-10 days after the second dose; and wherein each of the first, second and third doses comprises at least about 1.5×10⁹ NK cells.

Also provided herein is a method for the treatment of cancer, comprising: (a) selecting a subject for the treatment of cancer if cells of the cancer do not express CD58 or express a mutated form of CD58; (b) administering to the selected subject at least a first dosing cycle, wherein the first dosing cycle comprises a first dose of genetically engineered natural killer (NK) cells, a second dose of genetically engineered NK cells, and a third dose of genetically engineered NK cells, wherein the first dose is administered to a subject in need of cancer immunotherapy at a first time point, wherein the second dose is administered to the subject between 5-10 days after the first time point, wherein the third dose is administered to the subject between 5-10 days after the second dose; wherein each of the first, second and third doses comprise at least about 1.5×10⁹ NK cells, and wherein at least a portion of the genetically engineered NK cells is engineered to express a chimeric antigen receptor (CAR) that is directed against a CD19 tumor marker. In some embodiments, the first dosing cycle is initiated after the subject has undergone a lymphodepletion process to reduce native immune cell numbers. In some embodiments, the mutated form of CD58 comprises a loss of function.

Provided for herein, in several embodiments, is a method for the treatment of cancer, comprising administering to a subject having cancer a lymphodepletion regimen comprising at least two doses of cyclophosphamide and at least two doses of fludarabine, administering to the subject at least a first, a second, and a third dose of genetically engineered NK cells, wherein the first dose of genetically engineered NK cells is administered to the subject after the last dose fludarabine, wherein the second dose of genetically engineered NK cells is administered to the subject between 6-8 days after the first dose, wherein the third dose of genetically engineered NK cells is administered to the subject between 6-8 days after the second dose, wherein each of the first, second and third doses comprise about 1.5×10⁹ NK cells, and wherein the genetically engineered NK cells are allogeneic with respect to the subject and are engineered to express a chimeric antigen receptor (CAR) that binds CD19.

Also provided for herein is a method for the treatment of cancer, comprising administering to a subject having cancer a lymphodepletion regimen comprising at least two doses of cyclophosphamide and at least two doses of fludarabine, administering to the subject an agent that binds CD20, administering to the subject at least a first, a second, and a third dose of genetically engineered NK cells, wherein the first dose of genetically engineered NK cells is administered to the subject after the last dose fludarabine, wherein the second dose of genetically engineered NK cells is administered to the subject between 6-8 days after the first dose, wherein the third dose of genetically engineered NK cells is administered to the subject between 6-8 days after the second dose, wherein each of the first, second and third doses comprise at least 1.0×10⁹ NK cells, and wherein the genetically engineered NK cells are allogeneic with respect to the subject and are engineered to express a chimeric antigen receptor (CAR) that binds CD19. In several embodiments, about 1.5×10⁹ NK cells are administered at each of the three doses in a cycle.

Also provided for herein is the use of a population of engineered NK cells expressing a chimeric antigen receptor that targets CD19 for treating cancer, by administration of at least a first, a second, and a third dose of said genetically engineered NK cells, wherein the first dose of genetically engineered NK cells is administered to the subject after a final dose of a lymphodepletion process comprising at least two doses of cyclophosphamide and at least two doses of fludarabine, wherein the second dose is administered to the subject between 6-8 days after the first dose, wherein the third dose is administered to the subject between 6-8 days after the second dose, and wherein each of the first, second and third doses comprise about 1.5×10⁹ genetically engineered NK cells.

Additionally provided for herein is the use of a population of engineered NK cells expressing a chimeric antigen receptor that targets CD19 for treating cancer, by administration of at least a first, a second, and a third dose of said genetically engineered NK cells, wherein the first dose of genetically engineered NK cells is administered to the subject after a final dose of a lymphodepletion process comprising at least two doses of cyclophosphamide and at least two doses of fludarabine, wherein the first dose of genetically engineered NK cells is administered to the subject after administration of an agent that binds CD20, wherein the second dose is administered to the subject between 6-8 days after the first dose, wherein the third dose is administered to the subject between 6-8 days after the second dose, and wherein each of the first, second and third doses comprise at least 1.0×10⁹ genetically engineered NK cells. In several embodiments, each of the first, second and third doses comprise about 1.5×10⁹ genetically engineered NK cells.

In several embodiments, the NK cells are CAR-expressing NK cells. In several embodiments, each of the first, second and third doses comprises about 1.5×10⁹ CAR-expressing NK cells.

Also provided herein is use of genetically engineered natural killer (NK) cells expressing a chimeric antigen receptor (CAR) that is directed against an antigen associated with or expressed by cells of a cancer for treating a subject having the cancer, wherein cells of the cancer do not express CD58 or expresses a mutated form of CD58, and wherein the subject is relapsed and/or refractory to genetically engineered T cells expressing a CAR that is directed against the antigen. In some embodiments, the antigen is CD19.

Also provided herein is use of genetically engineered natural killer (NK) cells expressing a chimeric antigen receptor (CAR) that is directed against CD19 for treating a subject having a cancer, wherein the cancer does not express CD58 or expresses a mutated form of CD58.

Also provided herein is use of genetically engineered natural killer (NK) cells expressing a chimeric antigen receptor (CAR) that is directed against CD19 for treating a subject having a cancer, wherein the subject was selected for treatment based on having a cancer that does not express CD58 or expresses a mutated form of CD58.

In several embodiments, a dosing cycle (e.g., three doses of genetically engineered NK cells) is between about 14 days and about 35 days in duration. In several embodiments, the dosing cycle is about 21 days. In several embodiments, the dosing cycle is about 28 days.

In several embodiments, the lymphodepletion process comprises at least two doses of cyclophosphamide and at least two doses of fludarabine. In several embodiments, the lymphodepletion process comprises three doses of cyclophosphamide and three doses of fludarabine, wherein the first of the doses of cyclophosphamide and fludarabine are administered 5 days prior to the initiation of the dosing cycle, wherein the second of the doses of cyclophosphamide and fludarabine are administered 4 days prior to the initiation of the dosing cycle, and wherein the third of the doses of cyclophosphamide and fludarabine are administered 3 days prior to the initiation of the dosing cycle. In several embodiments, about two days are allowed to lapse between the third dose of cyclophosphamide and fludarabine and initiation of the dosing cycle.

In several embodiments, the cyclophosphamide is administered in an amount between about 100 and 600 mg/m² and the fludarabine is administered in an amount between about 10 and 60 mg/m². In several embodiments, the cyclophosphamide is administered in an amount between about 200 and 600 mg/m² and the fludarabine is administered in an amount between about 20 and 40 mg/m². In several embodiments, the cyclophosphamide is administered in an amount of about 300 mg/m². In several embodiments, the cyclophosphamide is administered in an amount of about 500 mg/m². In several embodiments, the fludarabine is administered in an amount of about 30 mg/m². In several embodiments, the cyclophosphamide is administered in an amount of about 300 mg/m² and the fludarabine is administered in an amount of about 30 mg/m².ln several embodiments, the cyclophosphamide is administered in an amount of about 500 mg/m² and the fludarabine is administered in an amount of about 30 mg/m².

In several embodiments, the first and second doses of genetically engineered NK cells are administered to the subject prior to the subject's native immune cell population recovering from the lymphodepletion process.

In several embodiments, the dosing regimen, methods and uses provided for are for treatment of a cancer or a tumor. In several embodiments, the dosing regimen, methods and uses provided for are for treatment of a cancer. In several embodiments, the dosing regimen, methods and uses provided for are for treatment of a blood cancer. In several embodiments, the cancer is a leukemia or a lymphoma. In several embodiments, the cancer is a B cell cancer. In several embodiments, the cancer is large B-cell lymphoma (LBCL). In several embodiments, the cancer is aggressive LBCL. In several embodiments, the cancer is a Non-Hodgkin lymphoma (NHL). In several embodiments, the cancer is diffuse large B-cell lymphoma (DLBCL), follicular lymphoma (FL), marginal zone lymphoma (MZL), mantle cell lymphoma (MCL), or B-cell acute lymphoblastic leukemia (B-ALL). In several embodiments, the cancer is diffuse large B-cell lymphoma (DLBCL). In several embodiments, the cancer is follicular lymphoma (FL). In several embodiments, the FL is high grade FL (e.g., FL grade 3b).

In several embodiments, the cancer is an indolent lymphoma (IL). In some embodiments, the FL is a grade 1, 2, or 3a FL. In several embodiments, the cancer is marginal zone lymphoma (MZL). In several embodiments, the cancer is mantle cell lymphoma (MCL). In several embodiments, the cancer is B-cell acute lymphoblastic leukemia (B-ALL). In several embodiments, the cancer is Waldenstr6 m macroglobulinemia (WM). In several embodiments, the cancer is chronic lymphocytic leukemia (CLL) or small lymphocytic lymphoma (SLL). In several embodiments, the cancer is CLL. In several embodiments, the cancer is SLL. In several embodiments, the cancer is primary mediastinal large B-cell lymphoma (PMBCL).

In several embodiments, the cancer is relapsed/refractory (R/R). In several embodiments, the cancer is a R/R NHL. In several embodiments, the cancer is a R/R B-ALL. In several embodiments, the cancer is a R/R LBCL. In several embodiments, the cancer is a R/R aggressive LBCL. In several embodiments, the cancer is a R/R MCL. In several embodiments, the cancer is a R/R IL. In several embodiments, the cancer is a R/R WM. In several embodiments, the cancer is a R/R CLL. In several embodiments, the cancer is a R/R SLL. In some embodiments, the subject has less than or equal to 5% peripheral blasts. In some embodiments, the subject has less than 5% peripheral blasts. In some embodiments, at the time of administration of the lymphodepleting therapy, the subject has less than or equal to 5% peripheral blasts. In some embodiments, at the time of administration of the first dose of the dosing cycle, the subject has less than or equal to 5% peripheral blasts. In some embodiments, at the time of administration of the lymphodepleting therapy, the subject has less than 5% peripheral blasts. In some embodiments, at the time of administration of the first dose of the dosing cycle, the subject has less than 5% peripheral blasts. In some embodiments, the subject does not have evidence of extramedullary disease. In some embodiments, the subject does not have other evidence of extramedullary disease. In some embodiments, the subject does not have evidence of extramedullary disease including lymphoblastic lymphoma. In some embodiments, the subject does not have other evidence of extramedullary disease including lymphoblastic lymphoma.

In several embodiments, the subject to be treated has received at least 1 but not more than 7 lines of previous therapy. In several embodiments, the subject has received at least 1 but not more than 4 lines of previous therapy. In several embodiments, the subject has received at least 1 line of previous therapy. In several embodiments, the subject has received at least 2 lines of previous therapy. In several embodiments, the subject has received at least 3 lines of previous therapy. In several embodiments, the subject has received at least 4 lines of previous therapy. In several embodiments, the subject has received 1 line of previous therapy. In several embodiments, the subject has received 2 lines of previous therapy. In several embodiments, the subject has received 3 lines of previous therapy. In several embodiments, the subject has received 4 lines of previous therapy.

In several embodiments, the subject to be treated has received a line of previous therapy. In several embodiments, the line of previous therapy comprises 1 previous line of therapy. In several embodiments, the line of previous therapy comprises 2 previous lines of therapy. In several embodiments, the line of previous therapy comprises 3 previous lines of therapy. In several embodiments, the line of previous therapy comprises 4 previous lines of therapy.

In several embodiments, a line of previous therapy comprises an anti-CD20 monoclonal antibody and a cytotoxic chemotherapy. In several embodiments, the cytotoxic therapy is anthracycline. A line of previous therapy comprises an inhibitor of Bruton's tyrosine kinase (BTKi). In several embodiments, the BTKi is ibrutinib. In several embodiments, a line of previous therapy comprises an inhibitor of Bcl-2. In several embodiments, the Bcl-2 inhibitor is venetoclax.

In several embodiments, a line of previous therapy comprises chimeric antigen receptor (CAR) T cells. In some embodiments, the subject is CAR T cell exposed. In some embodiments, the CAR T cells are autologous CAR T cells. In some embodiments, the subject is autologous CAR T cell exposed. In some embodiments, the CAR T cells target CD19. In some embodiments, a line of previous therapy comprises autologous anti-CD19 CAR T cells.

In several embodiments, a line of previous therapy does not comprise CAR T cells. In some embodiments, the subject is CAR T naive. In some embodiments, the subject is autologous CAR T naive.

In several embodiments, the first, second, and third doses of genetically engineered NK cells are administered to the subject within about 21 days of the first time point (e.g., the first dose). In several embodiments, the first, second, and third doses of genetically engineered NK cells are administered to the subject within about 14 days after the first time point (e.g., the first dose).

In several embodiments, the CAR comprises (a) an antigen-binding moiety that targets CD19; (b) a transmembrane domain; and (c) an intracellular signaling domain comprising an OX40 domain and a CD3zeta domain. In some embodiments, the antigen-binding moiety comprises a heavy chain variable region (VH) and a light chain variable region (VL), wherein the VH comprises a CDR-1, a CDR-2, and a CDR-3 comprising the amino acid sequences set forth in SEQ ID NOS: 45, 46, and 47, respectively; and the VL comprises a CDR-1, a CDR-2, and a CDR-3 comprising the amino acid sequences set forth in SEQ ID NOS: 48, 49, and 16, respectively. In some embodiments, the antigen-binding moiety comprises a heavy chain variable region (VH) and a light chain variable region (VL), wherein the VH comprises a CDR-1, a CDR-2, and a CDR-3 comprising the amino acid sequences set forth in SEQ ID NOS: 50, 23, and 24, respectively; and the VL comprises a CDR-1, a CDR-2, and a CDR-3 comprising the amino acid sequences set forth in SEQ ID NOS: 14, 15, and 16, respectively. In some embodiments, the antigen-binding moiety comprises a heavy chain variable region (VH) and a light chain variable region (VL), wherein the VH comprises the amino acid sequence set forth in SEQ ID NO: 21 and/or the VL comprises the amino acid sequence set forth in SEQ ID NO:13. In some embodiments, the antigen binding domain is an scFv comprising the amino acid sequence of SEQ ID NO:6.

In several embodiments, the genetically engineered NK cells express a chimeric receptor encoded by a polynucleotide having at least about 85%, at least about 90%, at least about 95%, or at least about 98% sequence identity to SEQ ID NO: 3. In several embodiments, genetically engineered NK cells express a chimeric receptor having at least about 85%, at least about 90%, at least about 95%, or at least about 98% sequence identity to SEQ ID NO: 4. In several embodiments, genetically engineered NK cells express a chimeric receptor comprising the amino acid sequence set forth in SEQ ID NO: 4. In several embodiments, genetically engineered NK cells express a chimeric receptor having at least about 85%, at least about 90%, at least about 95%, or at least about 98% sequence identity to SEQ ID NO: 43. In several embodiments, genetically engineered NK cells express a chimeric receptor comprising the amino acid sequence set forth in SEQ ID NO: 43. In several embodiments, the genetically engineered NK cells are also engineered to express membrane-bound interleukin 15 (mblL15). In several embodiments, the mblL15 has at least about 85%, at least about 90%, at least about 95%, or at least about 98% sequence identity to SEQ ID NO: 40. In several embodiments, the mblL15 comprises the amino acid sequence set forth in SEQ ID NO: 40. In several embodiments, the mblL15 has at least about 85%, at least about 90%, at least about 95%, or at least about 98% sequence identity to SEQ ID NO: 44. In several embodiments, the mblL15 comprises the amino acid sequence set forth in SEQ ID NO: 44.

In several embodiments, the dosing regimens, methods and uses do not result in cytokine release syndrome, immune effector cell-associated neurotoxicity syndrome (ICANS)/neurotoxicity, and/or graft versus host disease. In several embodiments, the dosing regimens, methods and uses do not result in cytokine release syndrome. In several embodiments, the dosing regimens, methods and uses do not result in immune effector cell-associated neurotoxicity syndrome (ICANS)/neurotoxicity. In several embodiments, the dosing regimens, methods and uses do not result in graft versus host disease.

In several embodiments, the genetically engineered NK cells are allogeneic with respect to the subject.

In several embodiments, the subject is a human. In several embodiments, the subject is at least 18 years of age.

In some embodiments, the subject has a 158V/158V CD16 genotype. In some embodiments, the subject has a 158F/1 58F CD16 genotype.

In some embodiments, the subject has an Eastern Cooperative Oncology Group (ECOG) Performance Status (PS) of less than or equal to 1. In some embodiments, the subject has an ECOG PS of less than or equal to 2. In some embodiments, the subject has an ECOG status of 0. In some embodiments, the subject has an ECOG status of 1. In some embodiments, the subject has an ECOG status of 2.

In some embodiments, if the subject exhibits a clinical response following a first dosing cycle, the subject is administered an additional dosing cycle. In some embodiments, if the subject exhibits a complete response (CR) following a first dosing cycle, the subject is administered an additional dosing cycle. In some embodiments, if the subject exhibits a clinical response following a dosing cycle and subsequently exhibits disease progression, the subject is administered an additional dosing cycle. In some embodiments, the subject is administered between one dosing cycle and five dosing cycles. In some embodiments, the subject is administered one dosing cycle. In some embodiments, the subject is administered two dosing cycles. In some embodiments, the subject is administered three dosing cycles. In some embodiments, the subject is administered four dosing cycles. In some embodiments, the subject is administered five dosing cycles. In some embodiments, the subject underdoes a lymphodepletion process prior to each dosing cycle.

In some embodiments, cells of the cancer do not express CD58 or express a mutated form of CD58. In some embodiments, cells of the cancer do not express CD58. In some embodiments, cells of the cancer express a mutated form of CD58. In some embodiments, prior to administration of the first dosing cycle to the subject, cells of the cancer are determined not to express CD58 or to express a mutated form of CD58. In some embodiments, prior to administration of the first dosing cycle to the subject, the subject has been selected for treatment with the dosing regimen based on cells of the cancer exhibiting a loss or mutation of CD58. In some embodiments, the mutation of CD58 comprises a loss of function mutation. In some embodiments, the mutation of CD58 is a loss of function mutation.

In some embodiments, one dose of genetically engineered NK cells is administered to the subject on an outpatient basis. In some embodiments, each dose of genetically engineered NK cells is administered to the subject on an outpatient basis.

### BRIEF DESCRIPTION OF THE DRAWINGS

FIG. 1A depicts a non-limiting schematic of a chimeric antigen receptor (CAR) construct (e.g., amino acid or nucleic acid) comprising a binding moiety for CD19.

FIG. 1B depicts a non-limiting schematic of a nucleic acid construct encoding the CAR of FIG. 1A as well as membrane-bound interleukin 15 (mblL15) (the mblL15 is expressed as a separate amino acid).

FIG. 2 depicts a non-limiting schematic of a 28-day cycle comprising three dosing events.

FIG. 3 shows tabulated data related to patient response rate to CD19-CAR NK cell administration according to a regimen provided for herein.

FIGS. 4A-4B show initial patient response data. FIG. 4A shows tumor imaging prior to inception of treatment. FIG. 4B shows tumor imaging after one cycle of a dosing regimen provided for herein.

FIG. 4C shows the maximum fold-change in cytokine levels per dosing cycle for subjects exhibiting complete response (CR) and subjects not exhibiting complete response (non-CR).

FIG. 4D shows the clinical responses of subjects treated with CD19 CAR-NK cells over time. CR, complete response; PR, partial response; SD, stable disease; PD, progressive disease.

FIGS. 5A-5B show data related to cytotoxicity of anti-CD20 antibodies against tumor cells. FIG. 5A shows cytotoxicity of rituximab (RTX) against Raji cells. FIG. 5B shows cytotoxicity of obinutuzumab (OBI) against Raji cells.

FIGS. 6A-6C show data related to cytotoxicity of combination therapies. FIG. 6A shows cytotoxicity of CD19-directed CAR NK cells against Raji cells when used in combination with 1 μg/ml rituximab (RTX) or obinutuzumab (OBI) in a 4-hour cytotoxicity assay. FIG. 6B shows cytotoxicity of CD19-directed CAR NK cells in combination with rituximab (RTX) against Raji cells. FIG. 6C shows cytotoxicity of CD19-directed CAR NK cells in combination with obinutuzumab (OBI) against Raji cells.

FIGS. 7A-7D show data related to cytotoxicity of combination therapies. FIG. 7A shows cytotoxicity of CD19-directed CAR NK cells in combination with rituximab (RTX; at 0.01 μg/ml) against Raji cells. FIG. 7B shows cytotoxicity of CD19-directed CAR NK cells in combination with obinutuzumab (OBI; at 0.01 μg/ml) against Raji cells. FIG. 7C shows cytotoxicity of CD19-directed CAR NK cells in combination with rituximab (RTX; at 10 μg/ml) against Raji cells with a rechallenge at day 3 of the coculture. FIG. 7D shows cytotoxicity of CD19-directed CAR NK cells in combination with obinutuzumab (OBI; at 10 μg/ml) against Raji cells with a rechallenge at day 3 of the coculture.

FIGS. 8A-8B show data related to determinations of whether a combination therapy functions in an antibody dependent cell-mediated cytotoxicity (ADCC)-dependent or ADCC-independent manner. FIG. 8A shows that the activity of CD19-directed CAR NK cells is independent of ADCC when targeting Raji cells. FIG. 8B shows that the activity of CD19-directed CAR NK cells is dependent on ADCC when targeting EHEB cells.

FIGS. 9A-9B show data related to the impact of anti-CD20 antibodies (1 μg/ml) on NK cell degranulation. FIG. 9A shows data related to the degranulation of NK cells, as measured by LAMP-1 staining. FIG. 9B shows LAMP-1 staining on NK cells stimulated with PMA and ionomycin.

FIGS. 10A-10B show data related to NK cell cytotoxicity against chronic lymphocytic leukemia primary cells with different CD16 polymorphisms. FIG. 10A shows data from a subject with the 158V CD16 polymorphism (158V/158V genotype) and cytotoxicity from a CD19 CAR NK cell alone, with rituximab (RTX; 1 μg/ml), or with mutated rituximab (RTX mutant; 1 μg/ml). FIG. 10B shows data from a subject with the 158F CD16 polymorphism (158F/158F genotype) and cytotoxicity from a CD19 CAR NK cell alone, with rituximab (1 μg/ml), or with mutated rituximab (1 μg/ml).

FIG. 11A shows cell surface expression of CD19 and CD58 in Nalm6 cells expressing CD19 and CD58 (WT), knocked out for CD19 (CD19KO), knocked out for CD58 (CD58KO), or knocked out for CD19 and CD58 (CD19KOCD58KO).

FIG. 11B shows CD2 binding in WT, CD19KO, CD58KO, and CD19KOCD58KO Nalm6 cells.

FIG. 11C shows cell surface expression of ULBP-4 in WT, CD19KO, CD58KO, and CD19KOCD58KO Nalm6 cells.

FIGS. 12A-B show the cytotoxicity of CD19 CAR T cells and CD19 CAR NK cells, respectively, against WT, CD19KO, and CD58KO Nalm6 target cells.

FIGS. 13A-C show the cytotoxicity of CD19 CAR NK cells against WT, CD19KO, and CD58KO Nalm6 target cells, respectively.

FIGS. 13D-F show the cytotoxicity of CD19 CAR T cells against WT, CD19KO, and CD58KO Nalm6 target cells, respectively.

FIG. 14 shows interferon-gamma production by CD19 CAR T cells co-cultured with WT, CD19KO, or CD58KO Nalm6 target cells.

FIG. 15A shows CD19KO Nalm6 tumor burden as measured by bioluminescence imaging (BLI) in mice treated with vehicle, CD19 CAR T cells, or CD19 CAR NK cells.

FIG. 15B shows WT and CD58KO Nalm6 tumor burden as measured by BLI in mice treated with vehicle or CD19 CAR T cells.

FIG. 15C shows WT and CD58KO Nalm6 tumor burden as measured by BLI in mice treated with vehicle or CD19 CAR NK cells.

FIG. 16 shows WT, CD19KO, and CD58KO Nalm6 tumor burden as measured by BLI in mice treated with CD19 CAR NK cells on Days 0, 7, and 14.

### DETAILED DESCRIPTION

Some embodiments of the methods and compositions provided herein relate to engineered immune cells and combinations of the same for use in immunotherapy. In several embodiments, the engineered cells are engineered in multiple ways, for example, to express a chimeric antigen receptor (CAR) that targets a tumor antigen.

The term “anticancer effect” refers to a biological effect which can be manifested by various means, including but not limited to, a decrease in tumor volume, a decrease in the number of cancer cells, a decrease in the number of metastases, an increase in life expectancy, decrease in cancer cell proliferation, decrease in cancer cell survival, and/or amelioration of various physiological symptoms associated with the cancerous condition.

### Cell Types

Some embodiments of the methods and compositions provided herein relate to a cell such as an immune cell. For example, an immune cell, such as an NK cell or a T cell, may be engineered to express a tumor-targeting CAR.

As opposed to traditional anti-cancer therapies such as surgical approaches, radiation therapy, chemotherapy, or combinations of these methods, targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In several embodiments, a patient's own immune cells, immune cells of a donor, or cells derived from a pluripotent cell, are modified to specifically eradicate that patient's type of cancer. Various types of immune cells can be used, such as T cells, Natural Killer (NK cells), or combinations thereof, as described in more detail below.

To facilitate cancer immunotherapies, there are also provided for herein polynucleotides, polypeptides, and vectors that encode CARs that comprise a target binding moiety (e.g., an extracellular binder of a ligand expressed by a cancer cell) operably coupled to a cytotoxic signaling complex. For example, some embodiments include a polynucleotide, polypeptide, or vector that encodes, for example an extracellular domain that is directed against a tumor marker, for example, CD19, to facilitate targeting of an immune cell to a CD19-expressing cancer and exerting cytotoxic effects on the cancer cell. Also provided are engineered immune cells (e.g., NK cells and/or T cells) expressing such CARs. Also provided are compositions (e.g., pharmaceutical compositions) comprising engineered immune cells (e.g., NK cells) expressing such CARs. Methods of treating cancer and other uses of such cells for cancer immunotherapy are also provided for herein.

### Engineered Cells for Immunotherapy

In several embodiments, cells of the immune system are engineered to have enhanced cytotoxic effects against target cells, such as tumor cells. For example, a cell of the immune system may be engineered to include a tumor-directed chimeric receptor and/or a tumor-directed CAR as described herein. In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those listed above or those that follow or are otherwise described herein may be engineered to express a chimeric antigen receptor, for example by providing to the cell a nucleic acid encoding the CAR. In several embodiments, the cells are optionally engineered to co-express a membrane-bound interleukin 15 (mblL15) domain. Thus, in several embodiments, the cells are engineered to express a CAR and a membrane-bound interleukin 15 (mblL15) domain. As discussed in more detail below, in several embodiments, the therapeutic cells, are further genetically modified enhance the cytotoxicity and/or persistence of the cells. In several embodiments, the genetic modification enhances the ability of the cell to resist signals emanating from the tumor microenvironment that would otherwise cause a reduced efficacy or shortened lifespan of the therapeutic cells.

### Monocytes for Immunotherapy

Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake cellular material, or entire cells, followed by digestion and destruction of the engulfed cellular material. In several embodiments, monocytes are used in connection with one or more additional engineered cells as disclosed herein. Several embodiments of the methods and compositions disclosed herein relate to monocytes engineered to express a CAR that targets a ligand on a tumor cell, for example, CD19, and optionally, a membrane-bound interleukin 15 (mblL15) domain. In several embodiments, monocytes are engineered to express a CAR that targets CD19. In several embodiments, monocytes are engineered to express a CAR that targets CD19 and a membrane-bound interleukin 15 (mblL15) domain.

### Lymphocytes for Immunotherapy

Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments, either from the same donor, or different donors). Several embodiments of the methods and compositions disclosed herein relate to lymphocytes engineered to express a CAR that targets a ligand on a tumor cell, for example, CD19, and optionally, a membrane-bound interleukin 15 (mblL15) domain. In several embodiments, lymphocytes are engineered to express a CAR that targets CD19. In several embodiments, lymphocytes are engineered to express a CAR that targets CD19 and a membrane-bound interleukin 15 (mblL15) domain.

### T Cells for Immunotherapy

T cells are distinguishable from other lymphocytes sub-types (e.g., B cells or NK cells) based on the presence of a T-cell receptor on the cell surface. T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a mixed pool of T cell subtypes is engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules. In several embodiments, there is provided a method of treating or preventing cancer or an infectious disease, comprising administering a therapeutically effective amount of T cells expressing the cytotoxic receptor complex and/or a homing moiety as described herein. In several embodiments, the engineered T cells are autologous cells, while in some embodiments, the T cells are allogeneic cells. Several embodiments of the methods and compositions disclosed herein relate to T cells engineered to express a CAR that targets a ligand on a tumor cell, for example, CD19, and optionally, a membrane-bound interleukin 15 (mblL15) domain. In several embodiments, T cells are engineered to express a CAR that targets CD19. In several embodiments, T cells are engineered to express a CAR that targets CD19 and a membrane-bound interleukin 15 (mblL15) domain.

### NK Cells for Immunotherapy

In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, the engineered NK cells are autologous cells, while in some embodiments, the NK cells are allogeneic cells. In several embodiments, the engineered NK cells are autologous cells. In several embodiments, the engineered NK cells are allogeneic cells. In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells). Several embodiments of the methods and compositions disclosed herein relate to NK cells engineered to express a CAR that targets a ligand on a tumor cell, for example, CD19, and optionally, a membrane-bound interleukin 15 (mblL15) domain. In several embodiments, immortalized NK cells are used and are subject to engineering, as disclosed herein. In several embodiments, primary NK cells are used and are subject to engineering, as disclosed herein. In some embodiments, the NK cells are derived from cell line NK-92. NK-92 cells are derived from NK cells, but lack major inhibitory receptors displayed by normal NK cells, while retaining the majority of activating receptors. Some embodiments of NK-92 cells described herein related to NK-92 cell engineered to silence certain additional inhibitory receptors, for example, SMAD3, allowing for upregulation of interferon-γ (IFNγ), granzyme B, and/or perforin production. Additional information relating to an NK-92 cell line is disclosed in WO 1998/49268 and U.S. Patent Application Publication No. 2002-0068044 and incorporated in their entireties herein by reference. NK-92 cells are used, in several embodiments, in combination with one or more of the other cell types disclosed herein. For example, in one embodiment, NK-92 cells are used in combination with NK cells as disclosed herein. In an additional embodiment, NK-92 cells are used in combination with T cells as disclosed herein. In several embodiments, NK cells are engineered to express a CAR that targets CD19. In several embodiments, NK cells are engineered to express a CAR that targets CD19 and a membrane-bound interleukin 15 (mblL15) domain.

### Hematopoietic Stem Cells for Cancer Immunotherapy

In some embodiments, hematopoietic stem cells (HSCs) are used in the methods of immunotherapy disclosed herein. In several embodiments, the cells are engineered to express a homing moiety and/or a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment. In several embodiments allogeneic HSCs are used, while in some embodiments, autologous HSCs are used. In several embodiments, HSCs are used in combination with one or more additional engineered cell type disclosed herein. Several embodiments of the methods and compositions disclosed herein relate to hematopoietic stem cells engineered to express a CAR that targets a ligand on a tumor cell, for example, CD19, and optionally, a membrane-bound interleukin 15 (mblL15) domain. In several embodiments, HSCs are engineered to express a CAR that targets CD19. In several embodiments, HSCs are engineered to express a CAR that targets CD19 and a membrane-bound interleukin 15 (mblL15) domain.

### Induced Pluripotent Stem Cells

In some embodiments, NK, T, or other immune cells derived from pluripotent stem cells (iPSCs) are used in the method of immunotherapy disclosed herein. In In some embodiments, induced pluripotent stem cells (iPSCs) are used in the method of immunotherapy disclosed herein. iPSCs are used, in several embodiments, to leverage their ability to differentiate and derive into non-pluripotent cells, including, but not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells comprising one or several genetic modifications at selected sites through differentiating iPSCs or less differentiated cells comprising the same genetic modifications at the same selected sites. In several embodiments, the iPSCs are used to generate iPSC-derived NK or T cells. In several embodiments, the cells are engineered to express a homing moiety and/or a cytotoxic receptor complex. In several embodiments, iPSCs are used in combination with one or more additional engineered cell type disclosed herein. Several embodiments of the methods and compositions disclosed herein relate to induced pluripotent stem cell-derived NK, T or other immune cells engineered to express a CAR that targets a ligand on a tumor cell, for example, CD19, and optionally, a membrane-bound interleukin 15 (mblL15) domain. In several embodiments, iPSCs are engineered to express a CAR that targets a ligand on a tumor cell, for example, CD19, and optionally, a membrane-bound interleukin 15 (mblL15) domain. In several embodiments, iPSCs are engineered to express a CAR that targets CD19. In several embodiments, iPSCs are engineered to express a CAR that targets CD19 and a membrane-bound interleukin 15 (mblL15) domain. In several embodiments, the engineered iPSCs are differentiated into NK, T, or other immune cells, such as for use in a composition or method provided herein.

### Chimeric Antigen Receptors

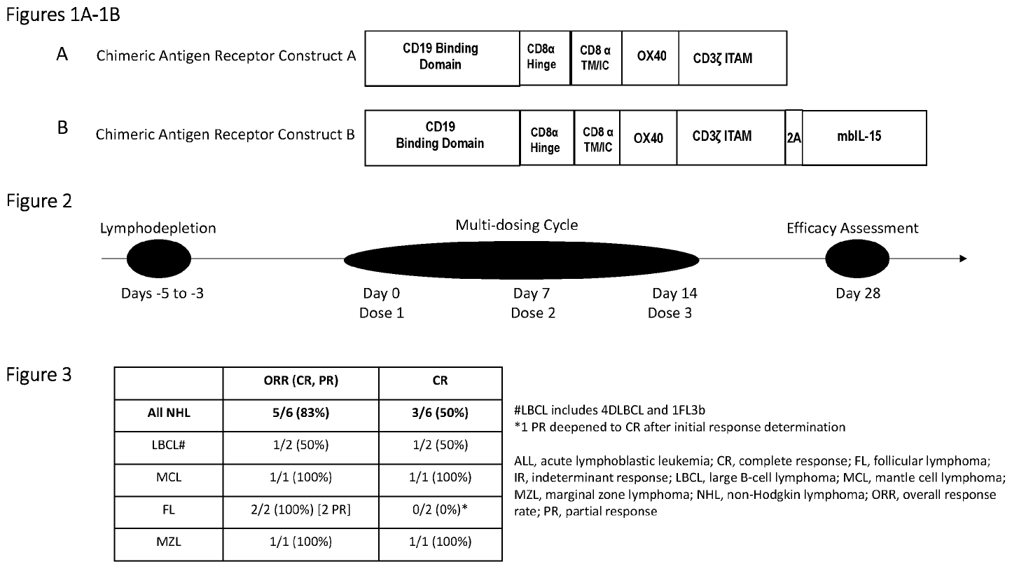

Some embodiments of the compositions and methods described herein relate to a chimeric receptor that includes an extracellular domain that comprises a tumor-binding domain (also referred to as an antigen-binding protein or antigen-binding domain) as described herein. The tumor-binding domain, depending on the embodiment, targets, for example, CD19. In several embodiments, the tumor-binding domain binds CD19. In several embodiments, the tumor-binding domain binds human CD19. The CAR construct according to several embodiments is schematically depicted in FIGS. 1A-1B. FIG. 1A shows a non-limiting CAR construct (e.g., amino acid or nucleic acid) comprising a CD19 binding domain, a CD8 alpha hinge and transmembrane domain, an OX40 co-stimulatory domain and a CD3 zeta signaling domain. FIG. 1B shows a non-limiting nucleic acid construct according to some embodiments, in which a membrane-bound interleukin15 (mblL15) is bicistronically encoded by the nucleic acid sequence encoding the CAR. In some embodiments, the mblL15 is bicistronically encoded by virtue of a bicistronic element between the nucleic acid sequence encoding the CAR and the nucleic acid sequence encoding the mblL15. It shall be appreciated that the translation of the nucleic acid of FIG. 1B would result in the CAR construct shown in FIG. 1A, with the mblL15 expressed separately on the cell. In some embodiments mblL15 is provided on a separate nucleic acid. In several embodiments, the CARs disclosed herein have the general structure of an extracellular antigen binding protein (that targets a cancer antigen, such as CD19), one or both of a hinge and a transmembrane domain, an optional costimulatory domain, and a signaling domain.

### Antigen-Binding Proteins

There are provided, in several embodiments, antigen-binding proteins. As used herein, the term “antigen-binding protein” shall be given its ordinary meaning, and shall also refer to a protein comprising an antigen-binding fragment that binds to an antigen and, optionally, a scaffold or framework portion that allows the antigen-binding fragment to adopt a conformation that promotes binding of the antigen-binding protein to the antigen. In some embodiments, the antigen is a cancer antigen or a fragment thereof. In some embodiments, the antigen-binding fragment comprises at least one CDR from an antibody that binds to the antigen. In some embodiments, the antigen-binding fragment comprises all three CDRs from the heavy chain of an antibody that binds to the antigen or from the light chain of an antibody that binds to the antigen. In still some embodiments, the antigen-binding fragment comprises all six CDRs from an antibody that binds to the antigen (three from the heavy chain and three from the light chain). In several embodiments, the antigen-binding fragment comprises one, two, three, four, five, or six CDRs from an antibody that binds to the antigen, and in several embodiments, the CDRs can be any combination of heavy and/or light chain CDRs. The antigen-binding fragment in some embodiments is an antibody fragment.

Non-limiting examples of antigen-binding proteins include antibodies, antibody fragments (e.g., an antigen-binding fragment of an antibody), antibody derivatives, and antibody analogs. Further specific examples include, but are not limited to, a single-chain variable fragment (scFv), a nanobody (e.g. VH domain of camelid heavy chain antibodies; VHH fragment,), a Fab fragment, a Fab′ fragment, a F(ab′)₂ fragment, a Fv fragment, a Fd fragment, and a complementarity determining region (CDR) fragment. These molecules can be derived from any mammalian source, such as human, mouse, rat, rabbit, or pig, dog, or camelid. Antibody fragments may compete for binding of a target antigen with an intact (e.g., native) antibody and the fragments may be roduced by the moIfication of Intact antibodies (e.g. enzymatic or chemical cleavage) or synthesized de novo using recombinant DNA technologies or peptide synthesis. The antigen-binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. In addition, peptide antibody mimetics (“PAMs”) can be used, as well as scaffolds based on antibody mimetics utilizing fibronectin components as a scaffold.

In some embodiments, the antigen-binding protein comprises one or more antibody fragments incorporated into a single polypeptide chain or into multiple polypeptide chains. For instance, antigen-binding proteins can include, but are not limited to, a diabody; an intrabody; a domain antibody (single VL or VH domain or two or more VH domains joined by a peptide linker); a maxibody (2 scFvs fused to Fc region); a triabody; a tetrabody; a minibody (scFv fused to CH3 domain); a peptibody (one or more peptides attached to an Fc region); a linear antibody (a pair of tandem Fd segments (VH-CH1-VH-CH1) which, together with complementary light chain polypeptides, form a pair of antigen binding regions); a small modular immunopharmaceutical; and immunoglobulin fusion proteins (e.g. IgG-scFv, IgG-Fab, 2scFv-IgG, 4scFv-IgG, VH-IgG, IgG-VH, and Fab-scFv-Fc).

In some embodiments, the antigen-binding protein has the structure of an immunoglobulin. As used herein, the term “immunoglobulin” shall be given its ordinary meaning, and shall also refer to a tetrameric molecule, with each tetramer comprising two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Within light and heavy chains, the variable (V) and constant regions I are joined by a “J” region of about 12 or more amino acids, with the heavy chain also including a “D” region of about 10 more amino acids. The variable regions of each light/heavy chain pair form the antibody binding site such that an intact immunoglobulin has two binding sites.

Immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

Human light chains are classified as kappa and lambda light chains. An antibody “light chain” refers to the smaller of the two types of polypeptide chains present in antibody molecules in their naturally occurring conformations. Kappa (κ) and lambda (λ) light chains refer to the two major antibody light chain isotypes. A light chain may include a polypeptide comprising, from amino terminus to carboxyl terminus, a single immunoglobulin light chain variable region (VL) and a single immunoglobulin light chain constant domain (CL).

Heavy chains are classified as mu (λ), delta (Δ), gamma (γ), alpha (α), and epsilon (E), and define the antibody's isotype as IgM, IgD, IgG, IgA, and IgE, respectively. An antibody “heavy chain” refers to the larger of the two types of polypeptide chains present in antibody molecules in their naturally occurring conformations, and which normally determines the class to which the antibody belongs. A heavy chain may include a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4).

The IgG-class is further divided into subclasses, namely, IgG1, IgG2, IgG3, and IgG4. The IgA-class is further divided into subclasses, namely IgA1 and IgA2. The IgM has subclasses including, but not limited to, IgM1 and IgM2. The heavy chains in IgG, IgA, and IgD antibodies have three domains (CH1, CH2, and CH3), whereas the heavy chains in IgM and IgE antibodies have four domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (e.g., between the light and heavy chain) and between the hinge regions of the antibody heavy chains.

In some embodiments, the antigen-binding protein is an antibody. The term “antibody”, as used herein, refers to a protein, or polypeptide sequence derived from an immunoglobulin molecule which specifically binds with an antigen. Antibodies can be monoclonal, or polyclonal, multiple or single chain, or intact immunoglobulins, and may be derived from natural sources or from recombinant sources. Antibodies can be tetramers of immunoglobulin molecules. The antibody may be “humanized”, “chimeric” or non-human. An antibody may include an intact immunoglobulin of any isotype, and includes, for instance, chimeric, humanized, human, and bispecific antibodies. An intact antibody will generally comprise at least two full-length heavy chains and two full-length light chains. Antibody sequences can be derived solely from a single species, or can be “chimeric,” that is, different portions of the antibody can be derived from two different species as described further below. Unless otherwise indicated, the term “antibody” also includes antibodies comprising two substantially full-length heavy chains and two substantially full-length light chains provided the antibodies retain the same or similar binding and/or function as the antibody comprised of two full length light and heavy chains. For example, antibodies having 1, 2, 3, 4, or 5 amino acid residue substitutions, insertions or deletions at the N-terminus and/or C-terminus of the heavy and/or light chains are included in the definition provided that the antibodies retain the same or similar binding and/or function as the antibodies comprising two full length heavy chains and two full length light chains. Examples of antibodies include monoclonal antibodies, polyclonal antibodies, chimeric antibodies, humanized antibodies, human antibodies, bispecific antibodies, and synthetic antibodies. There is provided, in some embodiments, monoclonal and polyclonal antibodies. As used herein, the term “polyclonal antibody” shall be given its ordinary meaning, and shall also refer to a population of antibodies that are typically widely varied in composition and binding specificity. As used herein, the term “monoclonal antibody” (“mAb”) shall be given its ordinary meaning, and shall also refer to one or more of a population of antibodies having identical sequences. Monoclonal antibodies bind to the antigen at a particular epitope on the antigen.

In some embodiments, the antigen-binding protein is a fragment or antigen-binding fragment of an antibody. The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CHI domains, linear antibodies, single domain antibodies such as sdAb (either vL or vH), camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23: 1126-1136, 2005). Antigen binding fragments can also be grafted into scaffolds based on polypeptides such as a fibronectin type Ill (Fn3) (see U.S. Pat. No. 6,703,199, which describes fibronectin polypeptide mini bodies). An antibody fragment may include a Fab, Fab′, F(ab′)₂, and/or Fv fragment that contains at least one CDR of an immunoglobulin that is sufficient to confer specific antigen binding to a cancer antigen (e.g., CD19). Antibody fragments may be produced by recombinant DNA techniques or by enzymatic or chemical cleavage of intact antibodies.

In some embodiments, Fab fragments are provided. A Fab fragment is a monovalent fragment having the VL, VH, CL and CH1 domains; a F(ab′)₂ fragment is a bivalent fragment having two Fab fragments linked by a disulfide bridge at the hinge region; a Fd fragment has the VH and CH1 domains; an Fv fragment has the VL and VH domains of a single arm of an antibody; and a dAb fragment has a VH domain, a VL domain, or an antigen-binding fragment of a VH or VL domain. In some embodiments, these antibody fragments can be incorporated into single domain antibodies, single-chain antibodies, maxibodies, minibodies, intrabodies, diabodies, triabodies, tetrabodies, v-NAR and bis-scFv. In some embodiments, the antibodies comprise at least one CDR as described herein.

There is also provided for herein, in several embodiments, single-chain variable fragments. As used herein, the term “single-chain variable fragment” (“scFv”) shall be given its ordinary meaning, and shall also refer to a fusion protein in which a VL and a VH region are joined via a linker (e.g., a synthetic sequence of amino acid residues) to form a continuous protein chain wherein the linker is long enough to allow the protein chain to fold back on itself and form a monovalent antigen binding site). For the sake of clarity, unless otherwise indicated as such, a “single-chain variable fragment” is not an antibody or an antibody fragment as defined herein. Diabodies are bivalent antibodies comprising two polypeptide chains, wherein each polypeptide chain comprises VH and VL domains joined by a linker that is configured to reduce or not allow for pairing between two domains on the same chain, thus allowing each domain to pair with a complementary domain on another polypeptide chain. According to several embodiments, if the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

In several embodiments, the antigen-binding protein comprises one or more CDRs. As used herein, the term “CDR” shall be given its ordinary meaning, and shall also refer to the complementarity determining region (also termed “minimal recognition units” or “hypervariable region”) within antibody variable sequences. The CDRs permit the antigen-binding protein to specifically bind to a particular antigen of interest. There are three heavy chain variable region CDRs (CDR-H1, CDR-H2 and CDR-H3) and three light chain variable region CDRs (CDR-L1, CDR-L2 and CDR-L3). The CDRs in each of the two chains typically are aligned by the framework regions to form a structure that binds specifically to a specific epitope or domain on the target protein. From N-terminus to C-terminus, naturally-occurring light and heavy chain variable regions both typically conform to the following order of these elements: FW1, CDR1, FW2, CDR2, FW3, CDR3, FW4. For heavy chain variable regions, the order is typically: FW-H1, CDR-H1, FW-H2, CDR-H2, FW-H3, CDR-H3, and FW-H4 from N-terminus to C-terminus. For light chain variable regions, the order is typically: FW-L1, CDR-L1, FW-L2, CDR-L2, FW-L3, CDR-L3, FW-L4 from N-terminus to C-terminus. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. Complementarity determining regions (CDRs) and framework regions (FR) of a given antibody may be identified using this system. Other numbering systems for the amino acids in immunoglobulin chains include IMGT® (the international ImMunoGeneTics information system; Lefranc et al, Dev. Comp. Immunol. 29:185-203; 2005) and Aho (Honegger and Pluckthun, J. Mol. Biol. 309(3):657-670; 2001). The binding domains disclosed herein may utilize CDRs defined according to any of these systems. For any given embodiment containing more than one CDR, the CDRs may be defined in accordance with any of Kabat, Chothia, extended, IMGT, Paratome, AbM, and/or conformational definitions, or a combination of any of the foregoing. Any of the CDRs, either separately or within the context of variable domains, can be interpreted by one of skill in the art under any of these numbering systems as appropriate. One or more CDRs may be incorporated into a molecule either covalently or noncovalently to make it an antigen-binding protein.

In several embodiments, the various components of the antigen-binding moiety are separated by a linker such as, a G₄S linker. In several embodiments, a G₄S linker comprises a sequence having at least 85%, 90%, 91%, 92%, 93%, 94%, 95%, 96%, 97%, 98%, 99%, or 100% identity to SEQ ID NO. 26. In several embodiments, a G₄S linker is encoded by a nucleic acid sequence having at least 85%, 90%, 91%, 92%, 93%, 94%, 95%, 96%, 97%, 98%, 99%, or 100% identity to SEQ ID NO. 25. In some embodiments, a (GₙS)ₓ linker is used (wherein the “n” represents the number of glycine residues followed by a serine, and “X” represents the number of GₙS repeats in a linker). Such linkers may also optionally be used elsewhere in the CAR, for example to provide the potential to separate the various component parts of the receptor complex along the polynucleotide, which can enhance expression, stability, and/or functionality of the receptor complex.

In several embodiments, the antigen-binding moiety comprises a binding moiety that targets CD19. In some embodiments, the antigen-binding moiety binds to CD19. In some embodiments, the antigen-binding moiety binds to human CD19. In several embodiments, the anti-CD19 binding moiety comprises a heavy chain variable region (VH) comprising heavy chain complementarity determining regions (CDRs) 1, 2, and 3 (HCDR1, HCDR2, and HCDR3), and a light chain variable region (VL) comprising light chain CDRs 1, 2, and 3 (LCDR1, LCDR2, and LCDR3).

In some embodiments, the HCDR1 comprises a sequence having at least 85%, 90%, 91%, 92%, 93%, 94%, 95%, 96%, 97%, 98%, 99%, or 100% identity to SEQ ID NO. 22. In some embodiments, the HCDR1 comprises the amino acid sequence set forth in SEQ ID NO. 22. In some embodiments, the HCDR2 comprises a sequence having at least 85%, 90%, 91%, 92%, 93%, 94%, 95%, 96%, 97%, 98%, 99%, or 100% identity to SEQ ID NO. 23. In some embodiments, the HCDR2 comprises the amino acid sequence set forth in SEQ ID NO. 23. In some embodiments, the HCDR3 comprises a sequence having at least 85%, 90%, 91%, 92%, 93%, 94%, 95%, 96%, 97%, 98%, 99%, or 100% identity to SEQ ID NO. 24. In some embodiments, the HCDR3 comprises the amino acid sequence set forth in SEQ ID NO. 24. In some embodiments, the HCDR1, HCDR2, and HCDR3, comprise the amino acid sequence set forth in SEQ ID NO. 22, 23, and 24, respectively. In some embodiments, the VH comprises a HCDR1, a HCDR2, and a HCDR3 comprising the amino acid sequence set forth in SEQ ID NOS. 22, 23, and 24, respectively. In some embodiments, the VH comprises a sequence having at least 85%, 90%, 91%, 92%, 93%, 94%, 95%, 96%, 97%, 98%, 99%, or 100% identity to SEQ ID NO. 21. In some embodiments, the VH comprises the amino acid sequence set forth in SEQ ID NO. 21. In some embodiments, the HCDR1 is encoded by a nucleic acid sequence having at least 85%, 90%, 91%, 92%, 93%, 94%, 95%, 96%, 97%, 98%, 99%, or 100% identity to SEQ ID NO. 18. In some embodiments, the HCDR2 is encoded by a nucleic acid sequence having at least 85%, 90%, 91%, 92%, 93%, 94%, 95%, 96%, 97%, 98%, 99%, or 100% identity to SEQ ID NO. 19. In some embodiments, the HCDR3 is encoded by a nucleic acid sequence having at least 85%, 90%, 91%, 92%, 93%, 94%, 95%, 96%, 97%, 98%, 99%, or 100% identity to SEQ ID NO. 20. In some embodiments, the VH is encoded by a nucleic acid sequence having at least 85%, 90%, 91%, 92%, 93%, 94%, 95%, 96%, 97%, 98%, 99%, or 100% identity to SEQ ID NO. 17.

In some embodiments, the LCDR1 comprises a sequence having at least 85%, 90%, 91%, 92%, 93%, 94%, 95%, 96%, 97%, 98%, 99%, or 100% identity to SEQ ID NO. 14. In some embodiments, the LCDR1 comprises the amino acid sequence set forth in SEQ ID NO. 14. In some embodiments, the LCDR2 comprises a sequence having at least 85%, 90%, 91%, 92%, 93%, 94%, 95%, 96%, 97%, 98%, 99%, or 100% identity to SEQ ID NO. 15. In some embodiments, the LCDR2 comprises the amino acid sequence set forth in SEQ ID NO. 15. In some embodiments, the LCDR3 comprises a sequence having at least 85%, 90%, 91%, 92%, 93%, 94%, 95%, 96%, 97%, 98%, 99%, or 100% identity to SEQ ID NO. 16. In some embodiments, the LCDR3 comprises the amino acid sequence set forth in SEQ ID NO. 16. In some embodiments, the LCDR1, LCDR2, and LCDR3, comprise the amino acid sequence set forth in SEQ ID NO. 14, 15, and 16, respectively. In some embodiments, the VL comprises a LCDR1, a LCDR2, and a LCDR3 comprising the amino acid sequence set forth in SEQ ID NOS. 14, 15, and 16, respectively. In some embodiments, the VL comprises a sequence having at least 85%, 90%, 91%, 92%, 93%, 94%, 95%, 96%, 97%, 98%, 99%, or 100% identity to SEQ ID NO. 13. In some embodiments, the VL comprises the amino acid sequence set forth in SEQ ID NO. 13. In some embodiments, the LCDR1 is encoded by a nucleic acid sequence having at least 85%, 90%, 91%, 92%, 93%, 94%, 95%, 96%, 97%, 98%, 99%, or 100% identity to SEQ ID NO. 10. In some embodiments, the LCDR2 is encoded by a nucleic acid sequence having at least 85%, 90%, 91%, 92%, 93%, 94%, 95%, 96%, 97%, 98%, 99%, or 100% identity to SEQ ID NO. 11. In some embodiments, the LCDR3 is encoded by a nucleic acid sequence having at least 85%, 90%, 91%, 92%, 93%, 94%, 95%, 96%, 97%, 98%, 99%, or 100% identity to SEQ ID NO. 12. In some embodiments, the VL is encoded by a nucleic acid sequence having at least 85%, 90%, 91%, 92%, 93%, 94%, 95%, 96%, 97%, 98%, 99%, or 100% identity to SEQ ID NO. 9.

In some embodiments, the VL comprises a CDR-L1, CDR-L2, and CDR-L3 comprising the amino acid sequences set forth in SEQ ID NOS. 14, 15, and 16, respectively; and the VH comprises a CDR-H1, a CDR-H2, and a CDR-H3 comprising the amino acid sequences set forth in SEQ ID NOS. 22, 23, and 24, respectively.

In some embodiments, the LCDR1 comprises a sequence having at least 85%, 90%, 91%, 92%, 93%, 94%, 95%, 96%, 97%, 98%, 99%, or 100% identity to SEQ ID NO. 48. In some embodiments, the LCDR1 comprises the amino acid sequence set forth in SEQ ID NO. 48. In some embodiments, the LCDR2 comprises a sequence having at least 85%, 90%, 91%, 92%, 93%, 94%, 95%, 96%, 97%, 98%, 99%, or 100% identity to SEQ ID NO. 49. In some embodiments, the LCDR2 comprises the amino acid sequence set forth in SEQ ID NO. 49. In some embodiments, the LCDR3 comprises a sequence having at least 85%, 90%, 91%, 92%, 93%, 94%, 95%, 96%, 97%, 98%, 99%, or 100% identity to SEQ ID NO. 16. In some embodiments, the LCDR3 comprises the amino acid sequence set forth in SEQ ID NO. 16. In some embodiments, the LCDR1, LCDR2, and LCDR3, comprise the amino acid sequence set forth in SEQ ID NO. 48, 49, and 16, respectively. In some embodiments, the VL comprises a LCDR1, a LCDR2, and a LCDR3 comprising the amino acid sequence set forth in SEQ ID NOS. 48, 49, and 16, respectively. In some embodiments, the VL comprises a sequence having at least 85%, 90%, 91%, 92%, 93%, 94%, 95%, 96%, 97%, 98%, 99%, or 100% identity to SEQ ID NO. 13. In some embodiments, the VL comprises the amino acid sequence set forth in SEQ ID NO. 13. In some embodiments, the VL comprises the CDR-L1, CDR-L2, and CDR-L3 of the VL sequence set forth in SEQ ID NO. 13. In some embodiments, the HCDR1 comprises a sequence having at least 85%, 90%, 91%, 92%, 93%, 94%, 95%, 96%, 97%, 98%, 99%, or 100% identity to SEQ ID NO. 45. In some embodiments, the HCDR1 comprises the amino acid sequence set forth in SEQ ID NO. 45. In some embodiments, the HCDR2 comprises a sequence having at least 85%, 90%, 91%, 92%, 93%, 94%, 95%, 96%, 97%, 98%, 99%, or 100% identity to SEQ ID NO. 46. In some embodiments, the HCDR2 comprises the amino acid sequence set forth in SEQ ID NO. 46. In some embodiments, the HCDR3 comprises a sequence having at least 85%, 90%, 91%, 92%, 93%, 94%, 95%, 96%, 97%, 98%, 99%, or 100% identity to SEQ ID NO. 47. In some embodiments, the HCDR3 comprises the amino acid sequence set forth in SEQ ID NO. 47. In some embodiments, the HCDR1, HCDR2, and HCDR3, comprise the amino acid sequence set forth in SEQ ID NO. 45, 46, and 47, respectively. In some embodiments, the VH comprises a HCDR1, a HCDR2, and a HCDR3 comprising the amino acid sequence set forth in SEQ ID NOS. 45, 46, and 47, respectively. In some embodiments, the VH comprises a sequence having at least 85%, 90%, 91%, 92%, 93%, 94%, 95%, 96%, 97%, 98%, 99%, or 100% identity to SEQ ID NO. 21. In some embodiments, the VH comprises the amino acid sequence set forth in SEQ ID NO. 21. In some embodiments, the VH comprises the CDR-H1, CDR-H2, and CDR-H3 of the VH sequence set forth in SEQ ID NO. 21.

In some embodiments, the VL comprises a CDR-L1, CDR-L2, and CDR-L3 comprising the amino acid sequences set forth in SEQ ID NOS. 48, 49, and 16, respectively; and the VH comprises a CDR-H1, a CDR-H2, and a CDR-H3 comprising the amino acid sequences set forth in SEQ ID NOS. 45, 46, and 47, respectively.

In some embodiments, the LCDR1 comprises a sequence having at least 85%, 90%, 91%, 92%, 93%, 94%, 95%, 96%, 97%, 98%, 99%, or 100% identity to SEQ ID NO. 14. In some embodiments, the LCDR1 comprises the amino acid sequence set forth in SEQ ID NO. 14. In some embodiments, the LCDR2 comprises a sequence having at least 85%, 90%, 91%, 92%, 93%, 94%, 95%, 96%, 97%, 98%, 99%, or 100% identity to SEQ ID NO. 15. In some embodiments, the LCDR2 comprises the amino acid sequence set forth in SEQ ID NO. 15. In some embodiments, the LCDR3 comprises a sequence having at least 85%, 90%, 91%, 92%, 93%, 94%, 95%, 96%, 97%, 98%, 99%, or 100% identity to SEQ ID NO. 16. In some embodiments, the LCDR3 comprises the amino acid sequence set forth in SEQ ID NO. 16. In some embodiments, the LCDR1, LCDR2, and LCDR3, comprise the amino acid sequence set forth in SEQ ID NO. 14, 15, and 16, respectively. In some embodiments, the VL comprises a LCDR1, a LCDR2, and a LCDR3 comprising the amino acid sequence set forth in SEQ ID NOS. 14, 15, and 16, respectively. In some embodiments, the VL comprises a sequence having at least 85%, 90%, 91%, 92%, 93%, 94%, 95%, 96%, 97%, 98%, 99%, or 100% identity to SEQ ID NO. 13. In some embodiments, the VL comprises the amino acid sequence set forth in SEQ ID NO. 13. In some embodiments, the VL comprises the CDR-L1, CDR-L2, and CDR-L3 of the VL sequence set forth in SEQ ID NO. 13. In some embodiments, the HCDR1 comprises a sequence having at least 85%, 90%, 91%, 92%, 93%, 94%, 95%, 96%, 97%, 98%, 99%, or 100% identity to SEQ ID NO. 50. In some embodiments, the HCDR1 comprises the amino acid sequence set forth in SEQ ID NO. 50. In some embodiments, the HCDR2 comprises a sequence having at least 85%, 90%, 91%, 92%, 93%, 94%, 95%, 96%, 97%, 98%, 99%, or 100% identity to SEQ ID NO. 23. In some embodiments, the HCDR2 comprises the amino acid sequence set forth in SEQ ID NO. 23. In some embodiments, the HCDR3 comprises a sequence having at least 85%, 90%, 91%, 92%, 93%, 94%, 95%, 96%, 97%, 98%, 99%, or 100% identity to SEQ ID NO. 24. In some embodiments, the HCDR3 comprises the amino acid sequence set forth in SEQ ID NO. 24. In some embodiments, the HCDR1, HCDR2, and HCDR3, comprise the amino acid sequence set forth in SEQ ID NO. 50, 23, and 24, respectively. In some embodiments, the VH comprises a HCDR1, a HCDR2, and a HCDR3 comprising the amino acid sequence set forth in SEQ ID NOS. 50, 23, and 24, respectively. In some embodiments, the VH comprises a sequence having at least 85%, 90%, 91%, 92%, 93%, 94%, 95%, 96%, 97%, 98%, 99%, or 100% identity to SEQ ID NO. 21. In some embodiments, the VH comprises the amino acid sequence set forth in SEQ ID NO. 21. In some embodiments, the VH comprises the CDR-H1, CDR-H2, and CDR-H3 of the VH sequence set forth in SEQ ID NO. 21.

In some embodiments, the VL comprises a CDR-L1, CDR-L2, and CDR-L3 comprising the amino acid sequences set forth in SEQ ID NOS. 14, 15, and 16, respectively; and the VH comprises a CDR-H1, a CDR-H2, and a CDR-H3 comprising the amino acid sequences set forth in SEQ ID NOS. 50, 23, and 24, respectively.

In some embodiments, the anti-CD19 binding moiety comprises a VH comprising a sequence having at least 85%, 90%, 91%, 92%, 93%, 94%, 95%, 96%, 97%, 98%, 99%, or 100% identity to SEQ ID NO. 21. In some embodiments, the anti-CD19 binding moiety comprises a VL comprising a sequence having at least 85%, 90%, 91%, 92%, 93%, 94%, 95%, 96%, 97%, 98%, 99%, or 100% identity to SEQ ID NO. 13. In some embodiments, the anti-CD19 binding moiety comprises a VH comprising a sequence having at least 85%, 90%, 91%, 92%, 93%, 94%, 95%, 96%, 97%, 98%, 99%, or 100% identity to SEQ ID NO. 21 and a VL comprising a sequence having at least 85%, 90%, 91%, 92%, 93%, 94%, 95%, 96%, 97%, 98%, 99%, or 100% identity to SEQ ID NO. 13. In some embodiments, the anti-CD19 binding moiety comprises a VH comprising the amino acid sequence set forth in SEQ ID NO. 21. In some embodiments, the anti-CD19 binding moiety comprises a VL comprising the amino acid sequence set forth in SEQ ID NO. 13. In some embodiments, the anti-CD19 binding moiety comprises a VH comprising the amino acid sequence set forth in SEQ ID NO. 21 and a VL comprising the amino acid sequence set forth in SEQ ID NO. 13.

Embodiments of the anti-CD19 binding moieties comprise various arrangements of the VH and VL disclosed herein. In some embodiments, the anti-CD19 binding moiety is a single-chain variable fragment (scFv). In some embodiments, the VH and VL are separated by a linker. In some embodiments, the linker comprises the sequence of SEQ ID NO: 26. In several embodiments, the linker is encoded by the nucleic acid of SEQ ID NO. 25. In some embodiments, the anti-CD19 binding moiety comprises a VH comprising the amino acid sequence set forth in SEQ ID NO. 21, a linker comprising the nucleic acid sequence set forth in SEQ ID NO. 26, and a VL comprising the amino acid sequence set forth in SEQ ID NO. 13.

In some embodiments of the anti-CD19 binding moieties, the VH is N-terminal of the VL. In some embodiments of the anti-CD19 binding moieties, the VL is N-terminal of the VH. In some embodiments, the anti-CD19 binding moiety comprises a sequence having at least 85%, 90%, 91%, 92%, 93%, 94%, 95%, 96%, 97%, 98%, 99%, or 100% identity to SEQ ID NO: 6. In some embodiments, the anti-CD19 binding moiety comprises the amino acid sequence set forth in SEQ ID NO: 6. In some embodiments, the anti-CD19 binding moiety is encoded by a nucleic acid comprising a sequence having at least 85%, 90%, 91%, 92%, 93%, 94%, 95%, 96%, 97%, 98%, 99%, or 100% identity to SEQ ID NO: 5.

In some embodiments, the antigen-binding proteins provided herein comprise one or more CDR(s) as part of a larger polypeptide chain. In some embodiments, the antigen-binding proteins covalently link the one or more CDR(s) to another polypeptide chain. In some embodiments, the antigen-binding proteins incorporate the one or more CDR(s) noncovalently. In some embodiments, the antigen-binding proteins may comprise at least one of the CDRs described herein incorporated into a biocompatible framework structure. In some embodiments, the biocompatible framework structure comprises a polypeptide or portion thereof that is sufficient to form a conformationally stable structural support, or framework, or scaffold, which is able to display one or more sequences of amino acids that bind to an antigen (e.g., CDRs, a variable region, etc.) in a localized surface region. Such structures can be a naturally occurring polypeptide or polypeptide “fold” (a structural motif), or can have one or more modifications, such as additions, deletions and/or substitutions of amino acids, relative to a naturally occurring polypeptide or fold. Depending on the embodiment, the scaffolds can be derived from a polypeptide of a variety of different species (or of more than one species), such as a human, a non-human primate or other mammal, other vertebrate, invertebrate, plant, bacteria or virus.

Depending on the embodiment, the biocompatible framework structures are based on protein scaffolds or skeletons other than immunoglobulin domains. In some such embodiments, those framework structures are based on fibronectin, ankyrin, lipocalin, neocarzinostain, cytochrome b, CP1 zinc finger, PST1, coiled coil, LACI-D1, Z domain and/or tendamistat domains.

### Transmembrane Domains

Some embodiments of the compositions and methods described herein relate to tumor antigen-directed CARs that comprise a transmembrane domain. Some embodiments include a transmembrane domain from NKG2D or another transmembrane protein. In several embodiments in which a transmembrane domain is employed, the portion of the transmembrane protein employed retains at least a portion of its normal transmembrane domain.

In several embodiments, however, the transmembrane domain comprises at least a portion of CD8, a transmembrane glycoprotein normally expressed on both T cells and NK cells. In several embodiments, the transmembrane domain comprises CD8α. In several embodiments, the transmembrane domain comprises a hinge and a transmembrane region. In several embodiments, the transmembrane domain comprises a CD8α hinge and a CD8α transmembrane region. In several embodiments, the transmembrane domain comprises a “hinge” (e.g., a CD8α hinge). In several embodiments, the “hinge” of CD8α is encoded by a nucleic acid sequence comprising the sequence set forth in SEQ ID NO: 27. In several embodiments, the “hinge” of CD8α is encoded by the nucleic acid sequence of SEQ ID NO: 27. In several embodiments, the CD8α hinge is truncated or modified and is encoded by a nucleic acid sequence that has at least 70%, at least 75%, at least 80%, at least 85%, at least 90%, at least 95% sequence identity with the CD8α having the sequence of SEQ ID NO: 27. In several embodiments, the “hinge” of CD8α comprises the amino acid sequence of SEQ ID NO: 28. In several embodiments, the CD8α can be truncated or modified, such that it has at least 70%, at least 75%, at least 80%, at least 85%, at least 90%, at least 95% sequence identity with the sequence of SEQ ID NO: 28.

In several embodiments, the transmembrane domain comprises a CD8α transmembrane region. In several embodiments, the CD8α transmembrane region is encoded by the nucleic acid sequence set forth in SEQ ID NO: 29. In several embodiments, the CD8α transmembrane region has the nucleic acid sequence of SEQ ID NO: 29. In several embodiments, the CD8α transmembrane region is truncated or modified and is encoded by a nucleic acid sequence that has at least 70%, at least 75%, at least 80%, at least 85%, at least 90%, at least 95% sequence identity with the CD8α having the sequence of SEQ ID NO: 29.

In several embodiments, the CD8α transmembrane region comprises the amino acid sequence of SEQ ID NO: 30. In several embodiments, the CD8α transmembrane region is truncated or modified and has at least 70%, at least 75%, at least 80%, at least 85%, at least 90%, at least 95% sequence identity with the CD8α having the sequence of SEQ ID NO: 30. In several embodiments, the transmembrane region includes a short portion of an intracellular region of a CD8α sequence (e.g., the C-terminal LTC (leucine-threonine-cysteine) motif in SEQ ID NO. 30).

In some embodiments, the transmembrane domain comprises a CD8α hinge and a CD8α transmembrane region. In some embodiments, the transmembrane domain comprises the amino acid sequences of SEQ ID NOS. 28 and 30. In some embodiments, the transmembrane domain comprises the amino acid sequence of SEQ ID NO. 51 or 52. In several embodiments, the transmembrane region includes a short portion of an intracellular region of a CD8α sequence (e.g., the C-terminal LTC (leucine-threonine-cysteine) motif in SEQ ID NO. 30). In some embodiments, the transmembrane domain comprises the amino acid sequence of SEQ ID NO. 52. In several embodiments, that motif is not present. In some embodiments, the transmembrane domain comprises the amino acid sequence of SEQ ID NO. 51.

### Signaling Domains

Some embodiments of the compositions and methods described herein relate to tumor antigen-directed CARs that include a signaling domain. For example, immune cells engineered according to several embodiments disclosed herein may comprise at least one subunit of the CD3 T cell receptor complex (or a fragment thereof). In several embodiments, the signaling domain comprises the CD3zeta subunit. In several embodiments, the CD3zeta is encoded by the nucleic acid sequence of SEQ ID NO: 33. In several embodiments, the CD3zeta can be truncated or modified, such that it is encoded by a nucleic acid that has at least 70%, at least 75%, at least 80%, at least 85%, at least 90%, at least 95% sequence identity with the CD3zeta having the sequence of SEQ ID NO: 33. In several embodiments, the CD3zeta domain comprises the amino acid sequence of SEQ ID NO: 34.

In several embodiments, the CD3zeta domain is truncated or modified and has at least 70%, at least 75%, at least 80%, at least 85%, at least 90%, at least 95% sequence identity with the CD3zeta domain having the sequence of SEQ ID NO: 34.

In several embodiments, unexpectedly enhanced signaling is achieved through the use of multiple signaling domains whose activities act synergistically. For example, in several embodiments, the signaling domain further comprises co-stimulatory intracellular signaling domain. For example, in several embodiments, the signaling domain further comprises an OX40 domain. In several embodiments, the OX40 domain is an intracellular signaling domain. In several embodiments, the OX40 intracellular signaling domain is encoded by the nucleic acid sequence of SEQ ID NO: 31. In several embodiments, the OX40 intracellular signaling domain can be truncated or modified, such that it is encoded by a nucleic acid that has at least 70%, at least 75%, at least 80%, at least 85%, at least 90%, at least 95% sequence identity with the sequence of SEQ ID NO: 31. In several embodiments, the OX40 intracellular signaling domain comprises the amino acid sequence of SEQ ID NO: 32. In several embodiments, the OX40 intracellular signaling domain is truncated or modified and has at least 70%, at least 75%, at least 80%, at least 85%, at least 90%, at least 95% sequence identity with the OX40 intracellular signaling domain having the sequence of SEQ ID NO: 32. In several embodiments, OX40 is used as the sole intracellular signaling domain in the construct, however, in several embodiments, OX40 can be used with one or more other signaling domains. For example, combinations of OX40 and CD3zeta are used in some embodiments. Thus, in some embodiments, the CAR comprises an anti-CD19 binding moiety, a CD8α transmembrane domain, a CD3zeta signaling domain, and an OX40 domain. In some embodiments, the CAR comprises the amino acid sequence set forth in SEQ ID NO. 43.

In some embodiments, an alternative co-stimulatory intracellular signaling domain is incorporated into the signaling domain. By way of further example, one or more of a CD28, OX40, or 4-1 BB intracellular signaling domain is used in combination with CD3zeta. By way of further example, combinations of CD28, OX40, 4-1 BB, and/or CD3zeta are used in some embodiments.

### Stimulatory Molecules

Some embodiments of the compositions and methods described herein relate to vectors encoding tumor antigen-directed CARs and a stimulatory molecule. In addition to the CAR comprising the various transmembrane domains and signaling domains (and the combination transmembrane/signaling domains), stimulatory molecules can be provided for expression by an immune cell, in several embodiments. These can be certain molecules that, for example, further enhance activity of the immune cells. Cytokines may be used in some embodiments. For example, certain interleukins, such as IL-2 and/or IL-15 as non-limiting examples, are used. In some embodiments, the immune cells for therapy are engineered to express such molecules as a secreted form. In additional embodiments, such stimulatory molecules are engineered to be membrane bound, acting as autocrine stimulatory molecules (or even as paracrine stimulators to neighboring cells).

In several embodiments, the NK cells disclosed herein are engineered to express interleukin 15 (IL15, IL-15). In some embodiments, the IL15 is expressed from a separate cassette on the construct comprising any one of the CARs disclosed herein. In some embodiments, the IL15 is expressed in the same cassette as any one of the CARs disclosed herein, optionally separated by a cleavage site, for example, a proteolytic cleavage site or a T2A, P2A, E2A, or F2A self-cleaving peptide cleavage site. In some embodiments, the IL-15 is expressed in the same cassette as any one of the CARs disclosed herein. In some embodiments, the IL15 is expressed in the same cassette as any one of the CARs disclosed herein, and the IL15 and CAR are separated by a cleavage site. In some embodiments, the cleavage site is a proteolytic cleavage site. In some embodiments, the cleavage site is a T2A, P2A, E2A, or F2A site. In some embodiments, the cleavage site is a T2A. In some embodiments, the IL15 is a membrane-bound IL15 (mblL15). In some embodiments, the mblL15 comprises a native IL15 sequence, such as a human native IL15 sequence, and at least one transmembrane domain. In some embodiments, the native IL15 sequence is encoded by a sequence having at least 85%, at least 90%, at least 95% sequence identity to SEQ ID NO: 37. In some embodiments, the native IL15 sequence comprises a peptide sequence having at least 85%, at least 90%, at least 95% sequence identity to SEQ ID NO: 38. In some embodiments, the native IL15 sequence comprises the amino acid sequence set forth in SEQ ID NO: 38.

In some embodiments, IL15 is membrane-bound by virtue of its coupling to at least one transmembrane domain. In some embodiments, the at least one transmembrane domain comprises a CD8 transmembrane domain. In some embodiments, the mblL15 may comprise additional components, such as a leader sequence and/or a hinge sequence. In some embodiments, the leader sequence is a CD8 leader sequence. In some embodiments, the hinge sequence is a CD8 hinge sequence. In some embodiments, the mbIL15 comprises an amino acid sequence having at least 85%, at least 90%, at least 95% sequence identity to SEQ ID NO: 44. In some embodiments, the mblL15 comprises the amino acid sequence set forth in SEQ ID NO: 44.

In some embodiments, the tumor antigen-directed CARs and/or tumor ligand-directed chimeric receptors are encoded by a polynucleotide that encodes for one or more cytosolic protease cleavage sites. Such sites are recognized and cleaved by a cytosolic protease, which can result in separation (and separate expression) of the various component parts of the receptor encoded by the polynucleotide. In some embodiments, the tumor antigen-directed CARs and/or tumor ligand-directed chimeric receptor are encoded by a polynucleotide that encodes for one or more self-cleaving peptides, for example a T2A cleavage site, a P2A cleavage site, an E2A cleavage site, and/or an F2A cleavage site. As a result, depending on the embodiment, the various constituent parts of an engineered cytotoxic receptor complex can be delivered to an NK cell or T cell in a single vector or by multiple vectors. Thus, as shown schematically, in the Figures, a construct can be encoded by a single polynucleotide, but also include a cleavage site, such that downstream elements of the constructs are expressed by the cells as a separate protein (as is the case in some embodiments with IL-15). In several embodiments, a T2A cleavage site is used. In several embodiments, a T2A cleavage site has the nucleic acid sequence of SEQ ID NO: 35. In several embodiments, a T2A cleavage site is encoded by the nucleic acid sequence of SEQ ID NO: 35. In several embodiments, T2A cleavage site can be truncated or modified, such that it is encoded by a nucleic acid sequence that has at least 70%, at least 75%, at least 80%, at least 85%, at least 90%, at least 95% sequence identity with the sequence of SEQ ID NO: 35. In several embodiments, the T2A cleavage site comprises the amino acid sequence of SEQ ID NO: 36. In several embodiments, the T2A cleavage site is truncated or modified and has at least 70%, at least 75%, at least 80%, at least 85%, at least 90%, at least 95% sequence identity with the T2A cleavage site having the sequence of SEQ ID NO: 36.

In several embodiments, NK cells are engineered to express membrane-bound interleukin 15 (mblL15). In such embodiments, mblL15 expression on the NK enhances the cytotoxic effects of the engineered NK cell by enhancing the proliferation and/or longevity of the NK cells. In several embodiments, the mblL15 is encoded by the same polynucleotide as the CAR. In some embodiments, IL15 is encoded by a polynucleotide comprising the sequence of SEQ ID NO: 37 and a sequence that encodes for a transmembrane domain. In some embodiments, IL15 comprises the amino acid sequence of SEQ ID NO: 38 coupled to an amino acid sequence of a transmembrane domain. In some embodiments, IL15 comprises the amino acid sequence of SEQ ID NO: 38 functionally coupled to an amino acid sequence of a transmembrane domain. In some embodiments, mblL15 is encoded by a polynucleotide comprising the sequence of SEQ ID NO: 39. In some embodiments, mblL15 comprises the amino acid sequence of SEQ ID NO: 44 (e.g., SEQ ID NO: 40). In some embodiments, mblL15 comprises the amino acid sequence of SEQ ID NO: 44. In several embodiments, mblL15 can be truncated or modified, such that it is encoded by a nucleic acid sequence that has at least 70%, at least 75%, at least 80%, at least 85%, at least 90%, at least 95% sequence identity with the sequence of SEQ ID NO: 39. In several embodiments, the mblL15 comprises the amino acid sequence of SEQ ID NO: 44 (e.g., SEQ ID NO: 40). In several embodiments, the mblL15 is truncated or modified and has at least 70%, at least 75%, at least 80%, at least 85%, at least 90%, at least 95% sequence identity with the sequence of SEQ ID NO: 44. In several embodiments, the mblL15 is truncated or modified and has at least 70%, at least 75%, at least 80%, at least 85%, at least 90%, at least 95% sequence identity with the sequence of SEQ ID NO: 44 (e.g., SEQ ID NO:40). Membrane-bound IL15 sequences are also described in PCT publications WO 2018/183385 and WO 2020/056045, each of which is hereby expressly incorporated by reference in its entirety.

### Chimeric Antigen Receptor Constructs

Some embodiments of the compositions and methods described herein relate to chimeric receptors, such as a CAR that targets (e.g., binds) CD19. The expression of these CARs in immune cells, such as genetically modified non-alloreactive T cells and/or NK cells, allows the targeting and destruction of particular target cells, such as cancerous cells. Non-limiting examples of such cytotoxic receptor complexes are discussed in more detail below.

In several embodiments, there is provided a polynucleotide (and the encoded amino acid) encoding a tumor binder/hinge-transmembrane domain/signaling complex. Various component parts may be used, according to embodiments disclosed herein.

In several embodiments, the polynucleotide further encodes an additional construct or molecule, for example a simulating molecule, like IL15. Thus, in some embodiments, the polynucleotide encodes a CAR and IL15 (e.g., membrane-bound IL15). In several embodiments, the polynucleotide therefore comprises, for example, a sequence encoding a T2A cleavage site. In some embodiments, the sequence encoding the T2A cleavage site is between the sequences encoding the CAR and IL15 (e.g., mblL15), such that the CAR and IL15 are bicistronically expressed.

In several embodiments, this CAR complex is encoded by a nucleic acid molecule comprising a sequence obtained from a combination of sequences disclosed herein, or comprises an amino acid sequence obtained from a combination of sequences disclosed herein. In several embodiments, the encoding nucleic acid sequence, or the amino acid sequence, comprises a sequence in accordance with one or more SEQ ID NOS as described herein, such as those included herein as examples of constituent parts. In several embodiments, the encoding nucleic acid sequence, or the amino acid sequence, comprises a sequence that shares at least about 90%, at least about 94%, at least about 95%, at least about 96%, at least about 97%, at least about 98%, or at least about 99%, sequence identity, homology and/or functional equivalence with a sequence resulting from the combination one or more SEQ ID NOS as described herein. It shall be appreciated that certain sequence variability, extensions, and/or truncations of the disclosed sequences may result when combining sequences, as a result of, for example, ease or efficiency in cloning (e.g., for creation of a restriction site). In several embodiments, the chimeric receptor comprises an amino acid sequence having at least 70%, 75%, 80%, 85%, 90%, 91%, 92%, 93%, 94%, 95%, 96%, 97%, 98%, 99%, or more, or a range defined by any two of the aforementioned percentages, identical to the sequence of one or more of the SEQ IDs provided for herein, or a portion thereof (e.g. a portion excluding the mblL15 sequence and/or self-cleaving peptide sequence).

In several embodiments, there is provided a polynucleotide encoding an CD19-binder/CD8α hinge/CD8α transmembrane domain/OX40/CD3zeta CAR (see FIG. 1A). The polynucleotide comprises or is composed of a CD19 binding domain, a CD8alpha hinge, a CD8α transmembrane domain, an OX40 domain, and a CD3zeta domain as described herein. In some embodiments, the CAR comprises, in order from N- to C-terminal, a CD19 binding domain, a CD8 alpha hinge, a CD8α transmembrane region, an OX40 co-stimulatory intracellular signaling domain, and a CD3zeta domain.

In several embodiments, this receptor complex is encoded by a nucleic acid molecule comprising the nucleic acid sequence of SEQ ID NO: 3. In several embodiments, the CD19 CAR is encoded by a nucleic acid that has at least 70%, at least 75%, at least 80%, at least 85%, at least 90%, at least 95% sequence identity with the sequence of SEQ ID NO: 3. In several embodiments, the CD19 CAR comprises the amino acid sequence of SEQ ID NO. 43 (e.g., SEQ ID NO: 4). In several embodiments, the CD19 CAR comprises the amino acid sequence of SEQ ID NO. 43. In several embodiments, the CD19 CAR comprises the amino acid sequence of SEQ ID NO: 4. In several embodiments, the CD19 CAR comprises an amino acid sequence that has at least 70%, at least 75%, at least 80%, at least 85%, at least 90%, at least 95% sequence identity with the sequence of SEQ ID NO: 43 (e.g., SEQ ID NO: 4). In several embodiments, while the CAR may vary from SEQ ID NO: 3, 4, or 43, the CAR retains, or in some embodiments, has enhanced, NK cell activating and/or cytotoxic function. Additionally, in several embodiments, this CD19 CAR construct can optionally be co-expressed on immune cells with, for example, mblL15, such as the mblL15 encoded by SEQ ID NO: 39. In several embodiments, the CD19 CAR is co-expressed with mblL15 encoded by a nucleic acid that has at least 70%, at least 75%, at least 80%, at least 85%, at least 90%, at least 95% sequence identity with the sequence of SEQ ID NO: 39. In several embodiments, the co-expressed mbIL15 comprises the amino acid sequence of SEQ ID NO: 44 (e.g., SEQ ID NO: 40). In several embodiments, the co-expressed mblL15 comprises the amino acid sequence of SEQ ID NO: 44. In several embodiments, the co-expressed mbIL15 comprises the amino acid sequence of SEQ ID NO: 40. In several embodiments, the co-expressed mblL15 has an amino acid sequence that has at least 70%, at least 75%, at least 80%, at least 85%, at least 90%, at least 95% sequence identity with the sequence of SEQ ID NO: 44 (e.g., SEQ ID NO: 40). Optionally the mblL15 can be introduced into a cell by a separate vector from that encoding the CAR. However, in several embodiments, the mblL15 is bicistronically encoded on the same nucleic acid sequence as the CD19 CAR. See, for example FIG. 1B. In several embodiments, the CD19 CAR and mblL15 are encoded by a nucleic acid of either SEQ ID NO: 1, 41 or 53. In several embodiments, the CD19 CAR and mblL15 are encoded by a nucleic acid of SEQ ID NO: 1. In several embodiments, the CD19 CAR and mblL15 are encoded by a nucleic acid of SEQ ID NO: 41. In several embodiments, the CD19 CAR and mblL15 are encoded by a nucleic acid of SEQ ID NO: 53. In several embodiments, the CD19 CAR and mblL15 are encoded by a nucleic acid that has at least 70%, at least 75%, at least 80%, at least 85%, at least 90%, at least 95% sequence identity with either the sequence of SEQ ID NO: 1, SEQ ID NO:41 or the sequence of SEQ ID NO: 53. In several embodiments, the CD19 CAR and mblL15 are encoded by a nucleic acid that has at least 70%, at least 75%, at least 80%, at least 85%, at least 90%, at least 95% sequence identity with the sequence of SEQ ID NO: 1. In several embodiments, the CD19 CAR and mblL15 are encoded by a nucleic acid that has at least 70%, at least 75%, at least 80%, at least 85%, at least 90%, at least 95% sequence identity with the sequence of SEQ ID NO: 41. In several embodiments, the CD19 CAR and mblL15 are encoded by a nucleic acid that has at least 70%, at least 75%, at least 80%, at least 85%, at least 90%, or at least 95% sequence identity with the sequence of SEQ ID NO: 53. In several embodiments, the co-encoded CD19 CAR and mblL15 have the amino acid sequence of SEQ NO: 2 or 42 (although they are ultimately expressed separately). In several embodiments, the co-encoded CD19 CAR and mblL15 comprise the amino acid sequence set forth in SEQ NO: 2. In several embodiments, the co-encoded CD19 CAR and mblL15 comprise the amino acid sequence set forth in SEQ NO: 42. In several embodiments, the co-encoded CD19 CAR and mblL15 comprises an amino acid sequence that has at least 70%, at least 75%, at least 80%, at least 85%, at least 90%, at least 95% sequence identity with the sequence of SEQ ID NO: 2 or 42 (although they are ultimately expressed separately). In several embodiments, the co-encoded CD19 CAR and mblL15 comprises an amino acid sequence that has at least 70%, at least 75%, at least 80%, at least 85%, at least 90%, at least 95% sequence identity with the sequence of SEQ ID NO: 2. In several embodiments, the co-encoded CD19 CAR and mblL15 comprises an amino acid sequence that has at least 70%, at least 75%, at least 80%, at least 85%, at least 90%, at least 95% sequence identity with the sequence of SEQ ID NO: 42. In several embodiments, the co-encoded CD19 CAR and mblL15 comprises the amino acid sequence of SEQ ID NO: 42.

Additional information about chimeric receptors for use in the presently disclosed methods and compositions can be found in U.S. Pat. Nos. 11,253,547, 11,141,436, 11,153,575, the entire contents of each of which is incorporated in its entirety by reference herein.

### Method of Treatment, Uses, Administration and Dosing

Some embodiments relate to a method of treating, ameliorating, inhibiting, or preventing cancer with a cell or immune cell comprising a chimeric antigen receptor, as disclosed herein. In some embodiments, the method includes treating or preventing cancer. In some embodiments, the method includes administering a therapeutically effective amount of immune cells expressing a tumor-directed chimeric antigen receptor as described herein. Examples of types of cancer that may be treated as such are described herein.

Disclosed herein are methods of treating cancer in a subject. In some embodiments, the methods comprise administering to the subject any one of the CD19 binding domains disclosed herein, any one of the CD19-directed CARs disclosed herein, or any one of the CAR-expressing cells disclosed herein, or any combination thereof.

Also disclosed herein are uses of any one of the CD19 binding domains disclosed herein, any one of the CD19-directed CARs disclosed herein, any one of the cells disclosed herein, or any combination thereof for the treatment of cancer.

Also disclosed herein are uses of any one of the CD19 binding domains disclosed herein, any one of the CD19-directed CARs disclosed herein, any one of the cells disclosed herein, or any combination thereof in the manufacture of a medicament for the treatment of cancer.

In certain embodiments, treatment of a subject with a genetically engineered cell(s) described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; and (xi) an enhancement, Improvement, supplementation, complementation, or augmentation of the prophylactic or therapeutic effect(s) of another therapy. Each of these comparisons are versus, for example, a different therapy for a disease, which includes a cell-based immunotherapy for a disease using cells that do not express the constructs disclosed herein. Advantageously, the engineered NK and/or T cells disclosed herein further enhance one or more of the above. In particular, it was surprisingly found that methods of treating subjects with dosing regimens as provided herein and described in the Working Examples (e.g., with NK cells engineered to express a CD19 CAR) result in unexpected efficacy and safety, including an efficacy and safety profile allowing for outpatient administration. Further, the inventors surprisingly discovered that the potency of engineered NK cells and compositions containing the same as described herein is not compromised when CD58 expression is absent in tumor cells, whereas the potency of engineered T cells is. CD58 is a co-stimulatory receptor that activates T cells and NK cells via its interaction with CD2 (Zhang et al., Front Immunol. (2021) 12: 705260). As CD58 loss or mutation is associated with poor responses to CD19 CAR-T therapies and reduced survival in clinical and preclinical models of blood cancers, the data provided herein demonstrate the unexpected finding that CD19 CAR NK cells do not lose potency in the same way that CD19 CAR T cells do against tumor cells lacking CD58 expression (Majzner et al., Blood (2020) 136 (Supplement 1): 53-54). Accordingly, and without wishing to be bound by theory, the engineered NK cells and related compositions, dosing regimens, methods, and uses as described herein, may provide superior antitumor activity than CD19 CAR T cells in patients with cancers exhibiting CD58 loss or mutation (e.g., a loss of function mutation).

Administration can be by a variety of routes, including, without limitation, intravenous, intra-arterial, subcutaneous, intramuscular, intrahepatic, intraperitoneal and/or local delivery to an affected tissue. The cells (in particular, NK cells and/or T cells) engineered to express a chimeric receptor complex described herein can be formulated for parenteral administration by injection, e.g., by bolus injection or infusion. As discussed in more detail, the cell therapies provided for herein can be delivered to a subject as a monotherapy, as a co-therapy with one or more additional anti-cancer agents or preparatory treatments.

Doses of immune cells such as NK and/or T cells can be readily determined for a given subject based on their body mass, disease type and state, and desired aggressiveness of treatment, but range, depending on the embodiments, from about 10⁹ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of immune cells such as NK and/or T cells is administered, for example between about 1×10⁸ cells/kg to about 1×10¹⁰ cells/kg.

In several embodiments, 1×10⁸ NK cells are administered (2×10⁶/kg for subject under 50 kg) three times over a 28-day cycle. In several embodiments, 3×10⁸ NK cells are administered (6×10⁶/kg for subject under 50 kg) three times over a 28-day cycle. In several embodiments, 1×10⁹ NK cells are administered (2×10⁷/kg for subject under 50 kg) three times over a 28-day cycle. In several embodiments, 1.5×10⁹ NK cells are administered (3×10⁷/kg for subject under 50 kg) three times over a 28-day cycle.

In several embodiments, 1×10⁸ CAR NK cells are administered (2×10⁶/kg for subject under 50 kg) three times over a 28-day cycle. In several embodiments, 3×10⁸ CAR NK cells are administered (6×10⁶/kg for subject under 50 kg) three times over a 28-day cycle. In several embodiments, 1×10⁹ CAR NK cells are administered (2×10⁷/kg for subject under 50 kg) three times over a 28-day cycle. In several embodiments, 1.5×10⁹ CAR NK cells are administered (3×10⁷/kg for subject under 50 kg) three times over a 28-day cycle.

Optionally, fewer doses (or more doses) may be used. For example, in several embodiments, 1.5×10⁸ NK cells are administered (3×10⁶/kg for a subject under 50 kg) two times over a 28-day cycle. In several embodiments, 4.5×10⁸ NK cells are administered two times over a 28-day cycle. In several embodiments, 1.5×10⁹ NK cells are administered two times over a 28-day cycle.

In several embodiments, 0.3×10⁹ NK cells are administered three times over a 28-day cycle. In several embodiments, 0.5×10⁹ NK cells are administered three times over a 28-day cycle. In several embodiments, 1.0×10⁹ NK cells are administered three times over a 28-day cycle. In several embodiments, 1.5×10⁹ NK cells are administered (3×10⁷/kg for a subject under 50 kg) three times over a 28-day cycle. In several embodiments, 3×10′ NK cells are administered three times over a 28-day cycle. In several embodiments, 1.5×1010 NK cells are administered three times over a 28-day cycle. In several embodiments, at least 4.5×10⁹ NK cells are administered over the cycle.

In several embodiments, 0.3×10⁹ CAR NK cells are administered three times over a 28-day cycle. In several embodiments, 0.5×10⁹ CAR NK cells are administered three times over a 28-day cycle. In several embodiments, 1.0×10⁹ CAR NK cells are administered three times over a 28-day cycle. In several embodiments, 1.5×10⁹ CAR NK cells are administered (3×10⁷/kg for a subject under 50 kg) three times over a 28-day cycle.

In several embodiments, 3×10⁹ CAR NK cells are administered three times over a 28-day cycle. In several embodiments, 1.5×10¹⁰ CAR NK cells are administered three times over a 28-day cycle. In several embodiments, at least 4.5×10⁹ CAR NK cells are administered over the cycle.

In some embodiments, a dose of NK cells of the dosing cycle is administered on an outpatient basis. In some embodiments, two doses of NK cells of the dosing cycle are administered on an outpatient basis. In some embodiments, each dose of NK cells of the dosing cycle is administered on an outpatient basis.

In several embodiments, the administration of engineered NK cells is preceded by one or more preparatory treatments. In several embodiments, the administration of engineered NK cells is preceded by lymphodepletion. In several embodiments, each dosing cycle is preceded by lymphodepletion. In several embodiments, a combination of chemotherapeutic agents is used for lymphodepletion. In several embodiments, a single chemotherapeutic agent is used for lymphodepletion. In several embodiments, wherein a combination of chemotherapeutic agents is used, agents with different mechanisms of actions are optionally used. In several embodiments, different classes of agents are optionally used.

In several embodiments, an antimetabolic agent is used. In several embodiments, the antimetabolic agent inhibits and/or prevents cell replication.

In several embodiments, cyclophosphamide, an alkylating agent that reduces tumor growth, is used in lymphodepletion. In several embodiments, the lymphodepletion comprises cyclophosphamide. In several embodiments, a dose of between about 200 and 600 mg/m² cyclophosphamide is administered, including doses of about 200 mg/m², about 225 mg/m², about 250 mg/m², about 275 mg/m², about 300 mg/m², about 325 mg/m², about 350 mg/m², about 400 mg/m², about 450 mg/m², about 475 mg/m², about 500 mg/m², about 525 mg/m², about 550 mg/m², about 600 mg/m², or about 700 mg/m², or any dose between those listed. In several embodiments, a dose of about 300 mg/m² cyclophosphamide is administered. In several embodiments, a dose of about 500 mg/m² cyclophosphamide is administered. In several embodiments, the dose of cyclophosphamide is given daily for days (e.g., prior to CAR-NK or CAR-T administration). In several embodiments, the dose of cyclophosphamide is given daily for at least about 2 days, about 3 days, about 4 days, about 5 days, about 6 days, or about 7 days (e.g., prior to CAR-NK or CAR-T administration). In several embodiments, the cyclophosphamide is given daily for 3 days. In several embodiments, if necessary, the dose can be split and given, for example, twice daily. In several embodiments, the cyclophosphamide is given daily for 3 days, starting 5 days prior to the first administration of a CD-19 CAR-expressing immune cell. In several embodiments, the cyclophosphamide is given at a dose of about 300 mg/m² daily for 3 days, starting 5 days prior to the first administration of a CD-19 CAR-expressing immune cell. In several embodiments, the cyclophosphamide is given at a dose of about 500 mg/m² daily for 3 days, starting 5 days prior to the first administration of a CD-19 CAR-expressing immune cell. In several embodiments, the cyclophosphamide is administered in combination with another agent.

In several embodiments, the additional agent is also an antimetabolite. In several embodiments, the additional agent inhibits one or more of DNA polymerase alpha, ribonucleotide reductase and/or DNA primase, thus inhibiting DNA synthesis. In several embodiments, the additional agent is fludarabine. In several embodiments, a dose of between about 5.0 mg/m²-about 200 mg/m² fludarabine is administered, including doses of about 5.0 mg/m², about 10.0 mg/m², about 15.0 mg/m², about 20.0 mg/m², about 25.0 mg/m², about 30.0 mg/m², about 35.0 mg/m², about 40.0 mg/m², about 45.0 mg/m², about 50.0 mg/m², about 60.0 mg/m², about 70.0 mg/m², about 80.0 mg/m², about 90.0 mg/m², about 100.0 mg/m², about 125.0 mg/m², about 150.0 mg/m², about 175.0 mg/m², about 200.0 mg/m², or any dose between those listed. In several embodiments, a dose of about 30 mg/m² fludarabine is administered. In several embodiments, the dose of fludarabine is given daily for at least about 2 days, about 3 days, about 4 days, about 5 days, about 6 days, or about 7 days. In several embodiments, the dose of fludarabine is given daily for about 3 days. In several embodiments, about 30 mg/m² fludarabine is given daily for about 3 days. In several embodiments, if necessary, the dose can be split and given, for example, twice daily.

In several embodiments, about 300 mg/m² cyclophosphamide and about 30 mg/m² fludarabine is each given daily for about 3 days. In several embodiments, prior to each dosing cycle, about 300 mg/m² cyclophosphamide and about 30 mg/m² fludarabine is each given daily for about 3 days. In several embodiments, about 500 mg/m² cyclophosphamide and about 30 mg/m² fludarabine is each given daily for about 3 days. In several embodiments, prior to each dosing cycle, about 500 mg/m² cyclophosphamide and about 30 mg/m² fludarabine is each given daily for about 3 days.

In several embodiments, the engineered cells expressing a CD19-directed CAR according to embodiments disclosed herein are administered in combination with an additional agent. For example, in several embodiments, another anticancer agent is administered (in addition to those used it the lymphodepletion process). In several embodiments, an antibody (e.g., monoclonal antibody or biosimilar) are used in conjunction with the engineered immune cells. In several embodiments, the antibody targets CD20. In several embodiments, the anti-CD20 antibody is rituximab. In some embodiments, biosimilar rituximab-abbs, rituximab-arrx, and/or rituximab-pvvr are used. In several embodiments, ocrelizumab, ofatumumab, obinutuzumab, ibritumomab, ibritumomab or combinations thereof are used. In several embodiments, the dose of the anti-CD20 antibody ranges from between about 150 mg/m² and about 500 mg/m², including about 150 mg/m², about 200 mg/m², about 250 mg/m², about 300 mg/m², about 350 mg/m², about 375 mg/m², about 400 mg/m², about 425 mg/m², about 450 mg/m², or about 500 mg/m² (or any dose between those listed). In several embodiments, the dose of the anti-CD20 antibody is about 375 mg/m². In some embodiments, the anti-CD20 antibody is rituximab and the dose is about 375 mg/m². In several embodiments, the dose of the anti-CD20 antibody is about 500 mg/m². In some embodiments, the anti-CD20 antibody is rituximab and the dose is about 500 mg/m². In several embodiments, the anti-CD20 antibody will be administered 1, 2, 3, or more times. In several embodiments, the anti-CD20 antibody will be administered 1, 2, 3, 4 or more days prior to administration of cells at the initial time point in a dosing cycle. In some embodiments, a single dose of 375 mg/m² rituximab is administered during the dosing cycle. In some embodiments, a single dose of 375 mg/m² rituximab is administered during the first dosing cycle, and a single dose of 500 mg/m² rituximab is administered during each subsequent dosing cycle. In some embodiments, the single dose of rituximab is administered prior to administration of the engineered immune cells, (e.g., about 3 days prior to administration). In some embodiments, the single dose of rituximab is administered about 3 days prior to administration of the engineered immune cells. In some embodiments, the single dose of rituximab is administered on about Day-3. In several embodiments, the anti-CD20 antibody will be administered 3 days prior to administration of cells.

In certain embodiments, a dose of a genetically engineered cell(s) described herein or composition thereof is administered to a subject every day, every other day, every couple of days, every third day, once a week, twice a week, three times a week, or once every two weeks. In other embodiments, two, three or four doses of a genetically engineered cell(s) described herein or composition thereof is administered to a subject every day, every couple of days, every third day, once a week or once every two weeks. In some embodiments, a dose(s) of a genetically engineered cell(s) described herein or composition thereof is administered for 2 days, 3 days, 5 days, 7 days, 14 days, 21 days, or 28 days. In certain embodiments, a dose of a genetically engineered cell(s) described herein or composition thereof is administered for 1 month, 1.5 months, 2 months, 2.5 months, 3 months, 4 months, 5 months, 6 months or more. In several embodiments, a dosing period is set and a certain number of doses is administered within that time period. For example, in several embodiments, a dosing cycle is 28 days in length with doses of engineered immune cells given on day 0, day 7, and day 14.

In several embodiments, a subject is subject to lymphodepletion at least one time prior to administration of genetically engineered cells as disclosed herein. In several embodiments, lymphodepletion is performed before one or more additional doses of engineered cells are administered. In several embodiments, a dosing cycle is used that comprises lymphodepletion followed by at least two doses of engineered cells as disclosed herein, with the two doses separated by a time interval. In several embodiments, the time interval is 2, 3, 4, 5, 6, 7, 8, 9, 10, 11, 12, 13, 14, 15, 16, 17, 18, 19, 20, 21, or more days (including intervals falling between the time marking a price interval since the last administration, e.g., 84 hours, or 3.5 days). In several embodiments, the dosing cycle itself is approximately 14, 21, 28, 35, 42 or more days. In several embodiments, three doses are administered, ˜1 week apart from each other. In several embodiments, two doses are administered ˜1 week apart from one another. In several embodiments, a subject receives a first dose on day 0 of the cycle, a second dose on day 7 of the cycle and a third dose on day 14 of the cycle. In several such embodiments, a 28-day cycle is used with primary outcome measures evaluated at day 28 (see e.g., FIG. 2). In several embodiments, lymphodepletion is performed prior to the inception of each dosing cycle, if subsequent dosing cycles are required (e.g., the subject requires further treatment). For example, in several embodiments, a subject undergoes lymphodepletion, receives a plurality of doses of engineered cells according to a cycle, is evaluated at the end of the cycle time and, if deemed necessary undergoes a second lymphodepletion followed by a second dosing cycle. In several embodiments, fludarabine/cyclophosphamide is used to achieve lymphodepletion. In several embodiments, cyclophosphamide (500 mg/m²) and fludarabine (30 mg/m²) are administered daily for 5 days. Depending on the embodiment, different concentrations may be used. In such embodiments where multiple dosing cycles are used, a first and a second dosing cycle need not be the same (e.g., a first cycle may have 2 doses, while a second uses three doses). Depending on the subject 1, 2, 3, 4, 5, 6, 7, 8, 9, 10, or more dosing cycles are performed.

Advantageously, in several embodiments, the therapies and dosing regimens provided for herein provide effective anti-cancer treatment without certain CAR-T cell toxicities, such as cytokine release syndrome, immune effector cell-associated neurotoxicity syndrome (ICANS) or neurotoxicity, or graft-versus host disease. In several embodiments, complete remission is achieved. In several embodiments, complete response (CR) is achieved. In several embodiments, partial response (PR) is achieved. In several embodiments, stable disease (SD) or limited progression of disease is accomplished.

Clinical outcomes can be assessed by any of the methods known in the art, including based on the Lugano classification with lymphoma response to immunomodulatory therapy criteria (LYRIC) refinement for subjects with non-Hodgkin lymphoma (NHL); the 2018 International Workshop on Chronic Lymphocytic Leukemia (iwCLL) guidelines for subjects with chronic lymphocytic leukemia (CLL) or small lymphocytic lymphoma (SLL); Version 1.2020 National Comprehensive Cancer Network (NCCN) for subjects with B-cell acute lymphoblastic leukemia (B-ALL); or 6^{th} International Workshop on Waldenstr6 m macroglobulinemia (WM) for subjects with WM. See Cheson et al., Blood (2016) 128(21):2489-96; Cheson et al., J Clin Oncol (2014) 32(27):3059-68; Hallek et al., Blood (2018) 131(25):2745-60; NCCN Guidelines for Acute Lymphoblastic Leukemia 1.2020; and Owen et al., Br J Haematol (2013) 160(2):171-6.

In some embodiments, also provided herein are nucleic acid and amino acid sequences that have sequence identity and/or homology of at least 80%, 85%, 90%, 95%, 96%, 97%, 98%, 99% (and ranges therein) as compared with the respective nucleic acid or amino acid sequences of SEQ ID NOS: 1-44 (or combinations of two or more of SEQ ID NOS: 1-44) and that also exhibit one or more of the functions as compared with the respective SEQ ID NOS: 1-44 (or combinations of two or more of SEQ ID NOS: 1-44) including but not limited to, (i) enhanced proliferation, (ii) enhanced activation, (iii) enhanced cytotoxic activity against cells presenting ligands to which NK cells harboring receptors encoded by the nucleic acid and amino acid sequences bind, (iv) enhanced homing to tumor or infected sites, (v) reduced off target cytotoxic effects, (vi) enhanced secretion of immunostimulatory cytokines and chemokines (including, but not limited to IFNg, TNFa, IL-22, CCL3, CCL4, and CCL5), (vii) enhanced ability to stimulate further innate and adaptive immune responses, and (viii) combinations thereof.

Additionally, in several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

In several embodiments, polynucleotides encoding the disclosed cytotoxic receptor complexes are mRNA. In some embodiments, the polynucleotide is DNA.

In some embodiments, the polynucleotide is operably linked to at least one regulatory element for the expression of the cytotoxic receptor complex.

Additionally provided, according to several embodiments, is a vector comprising the polynucleotide encoding any of the polynucleotides provided for herein, wherein the polynucleotides are optionally operatively linked to at least one regulatory element for expression of a cytotoxic receptor complex. In several embodiments, the vector is a retrovirus.

Further provided herein are engineered immune cells (such as NK and/or T cells) comprising the polynucleotide, vector, or cytotoxic receptor complexes as disclosed herein. Further provided herein are engineered NK cells comprising the polynucleotide, vector, or cytotoxic receptor complexes as disclosed herein. Further provided herein are compositions comprising a mixture of engineered immune cells (such as NK cells and/or engineered T cells), each population comprising the polynucleotide, vector, or cytotoxic receptor complexes as disclosed herein. Further provided herein are compositions comprising engineered NK cells comprising the polynucleotide, vector, or cytotoxic receptor complexes as disclosed herein.

### Subjects

Some embodiments of the compositions and methods described herein relate to administering immune cells comprising a chimeric antigen receptor to a subject with cancer.

In several embodiments, the subject has large B-cell lymphoma (LBCL). In several embodiments, the subject has aggressive LBCL. In several embodiments, the subject has Non-Hodgkin lymphoma (NHL). In several embodiments, the subject has diffuse large B-cell lymphoma (DLBCL), follicular lymphoma (FL), marginal zone lymphoma (MZL), mantle cell lymphoma (MCL), or B-cell acute lymphoblastic leukemia (B-ALL). In several embodiments, the subject has diffuse large B-cell lymphoma (DLBCL). In several embodiments, the subject has follicular lymphoma (FL). In several embodiments, the subject has high grade FL (e.g., FL grade 3b). In several embodiments, the subject has indolent lymphoma (IL). In several embodiments, the subject has grade 1, 2, or 3a FL. In several embodiments, the subject has marginal zone lymphoma (MZL). In several embodiments, the subject has mantle cell lymphoma (MCL). In several embodiments, the subject has B-cell acute lymphoblastic leukemia (B-ALL). In several embodiments, the subject has Waldenstr6 m macroglobulinemia (WM). In several embodiments, the subject has Chronic lymphocytic leukemia (CLL) or small lymphocytic lymphoma (SLL). In several embodiments, the subject has CLL. In several embodiments, the subject has SLL. In several embodiments, the subject has primary mediastinal large B cell lymphoma (PMBCL). In some embodiments, the cancer is a relapsed/refractory (r/r) cancer.

In some embodiments, the subject has marrow-localized disease (e.g., 55% peripheral blasts without other evidence of extramedullary disease including lymphoblastic lymphoma). In some embodiments, the subject has 55% peripheral blasts. In some embodiments, the subject has <5% peripheral blasts. In some embodiments, the subject has r/r B-ALL. In some embodiments, the subject has r/r B-ALL with 55% peripheral blasts. In some embodiments, the subject has r/r B-ALL with <5% peripheral blasts. In some embodiments, the subject does not have evidence of extramedullary disease. In some embodiments, the subject does not have other evidence of extramedullary disease. In some embodiments, the subject does not have evidence of extramedullary disease including lymphoblastic lymphoma. In some embodiments, the subject does not have other evidence of extramedullary disease including lymphoblastic lymphoma.

In some embodiments, the subject has measurable disease as defined by any of the methods for diagnosing and staging known in the art, including WHO 2016 classification for r/r B cell NHL or B-ALL (Quintanilla-Martinez, Hematological Oncology (2017) 35:37-4); Lugano classification for NHL (Cheson et al., J. Clin. Oncol (2014) 32(27):3059-68); iwCLL for CLL and SLL (Hallek et al., Blood (2018) 131(25):2745-60); and Second International Workshop on Waldenstr6 m Macroglobulinemia for WM (Owen et al., Semin Oncol (2003) 30(2):110-15).

In some embodiments, the subject has been treated with a previous line of therapy. In some embodiments, the subject is relapsed/refractory (R/R) to a previous line of therapy. In some embodiments, the previous line of therapy comprises one previous line of therapy. In some embodiments, the subject has MCL, the previous line of therapy is one previous line of therapy, and the one previous line of therapy is not CAR T cells. In some embodiments, the subject has WM, and the previous line of therapy is one previous line of therapy. In some embodiments, the previous line of therapy comprises two previous lines of therapy. In some embodiments, the previous line of therapy comprises three previous lines of therapy. In some embodiments, the previous line of therapy comprises four previous lines of therapy. In some embodiments, the subject did not respond to or relapsed within 12 months of completion of the prior line of therapy. In some embodiments, the subject did not respond to the prior line of therapy. In some embodiments, the subject relapsed within 12 months of completion of the prior line of therapy.

In some embodiments, the previous line of therapy comprises an inhibitor of Bruton's tyrosine kinase (BTKi). In some embodiments, the subject has been previously treated with a BTKi. In some embodiments, the subject is R/R to a BTKi. In some embodiments, the BTKi comprises ibrutinib. In some embodiments, the BTKi is ibrutinib. In some embodiments, the subject has been previously treated with ibrutinib. In some embodiments, the subject is R/R to ibrutinib.

In some embodiments, the previous line of therapy comprises a tyrosine kinase inhibitor. In some embodiments, the subject has Philadelphia chromosome (Ph+) B-ALL and the previous line of therapy comprises a tyrosine kinase inhibitor.

In some embodiments, the previous line of therapy comprises a Bcl-2 inhibitor. In some embodiments, the subject has been previously treated with a Bcl-2 inhibitor.

In some embodiments, the subject is R/R to a Bcl-2 inhibitor. In some embodiments, the Bcl-2 inhibitor comprises venetoclax. In some embodiments, the Bcl-2 inhibitor is venetoclax. In some embodiments, the subject has been previously treated with venetoclax. In some embodiments, the subject is R/R to venetoclax.

In some embodiments, the previous line of therapy comprises a BTKi and a Bcl-2 inhibitor. In some embodiments, the subject has been previously treated with a BTKi and a Bcl-2 inhibitor. In some embodiments, the subject is R/R to a BTKi and a Bcl-2 inhibitor. In some embodiments, the BTKi is ibrutinib. In some embodiments, the Bcl-2 inhibitor is venetoclax.

In some embodiments, the previous line of therapy comprises a CD20-targeted therapy and a cytotoxic chemotherapy (e.g., anthracycline). In some embodiments, the CD20-targeted therapy is an anti-CD20 antibody. In some embodiments, the anti-CD20 antibody is an anti-CD20 monoclonal antibody. In some embodiments, the anti-CD20 antibody comprises rituximab. In some embodiments, the anti-CD20 antibody is rituximab. In some embodiments, the cytotoxic chemotherapy comprises anthracycline. In some embodiments, the cytotoxic chemotherapy is anthracycline. In some embodiments, the subject has been previously treated with an anti-CD20 monoclonal antibody and a cytotoxic therapy (e.g., anthracycline). In some embodiments, the subject is R/R to an anti-CD20 monoclonal antibody and a cytotoxic therapy (e.g., anthracycline). In some embodiments, if the previous line of therapy comprises a CD20-targeted therapy, cells of the cancer are CD20+(e.g., as assessed locally).

In some embodiments, the previous line of therapy comprises a CD19-directed therapy. In some embodiments, the subject has been previously treated with a CD19-directed therapy. In some embodiments, the previous line of therapy comprises chimeric antigen receptor (CAR) T cells. In some embodiments, the subject has been previously treated with CAR T cells (CAR T exposed). In some embodiments, the subject has been previously treated with anti-CD19 CAR T cells. In some embodiments, the CAR T cells are autologous CAR T cells. In some embodiments, the subject has been previously treated with autologous anti-CD19 CAR T cells. In some embodiments, if the previous line of therapy comprises a CD19-directed therapy, cells of the cancer are CD19+(e.g., as assessed locally).

In some embodiments, the previous line of therapy does not comprise a CD19-directed therapy. In some embodiments, the subject has not been previously treated with a CD19-directed therapy. In some embodiments, the previous line of therapy does not comprise chimeric antigen receptor (CAR) T cells. In some embodiments, the subject has not been previously treated with CAR T cells (CAR T naive). In some embodiments, the subject has not been previously treated with autologous CAR T cells. In some embodiments, the subject has not been previously treated with anti-CD19 CAR T cells. In some embodiments, the subject has not been previously treated with autologous anti-CD19 CART T cells.

In some embodiments, the subject is a human. In some embodiments, the subject is an adult. In some embodiments, the subject is at least 18 years of age.

In some embodiments, the subject has an Eastern Cooperative Oncology Group Performance Status (ECOG) of 0, 1, or 2. In some embodiments, the subject has an Eastern Cooperative Oncology Group Performance Status (ECOG) of 0 or 1. In some embodiments, the subject has an ECOG of 0. In some embodiments, the subject has an ECOG of 1. In some embodiments, the subject has an ECOG of 2.

In some embodiments, the subject has adequate organ function. In some embodiments, adequate organ function comprises a platelet count≥30,000/μL. In some embodiments, adequate organ function comprises serum creatinine value≤1.5× upper limit of normal (ULN). In some embodiments, adequate organ function comprises total bilirubin value≤1.5×ULN or ≤3.0×ULN for subjects with hereditary benign hyperbilirubinemia. In some embodiments, adequate organ function comprises aspartate aminotransferase (AST)/serum glutamic-oxaloacetic transaminase (SGOT) value≤3×ULN and alanine aminotransferase (ALT)/serum glutamic pyruvic transaminase (SGPT) value≤3×ULN. In some embodiments, adequate organ function comprises baseline international normalized ratio (INR) 52 or activated partial thromboplastin time (aPTT) of≤2 times ULN. In some embodiments, adequate organ function comprises, the subject does not require oxygen therapy.

In some embodiments, the subject does not have Burkitt lymphoma. In some embodiments, the subject does not have primary central nervous system (CNS) lymphoma. In some embodiments, the subject does not have Richter's transformation to Hodgkin lymphoma.

### Cancer Types

Some embodiments of the compositions and methods described herein relate to administering immune cells comprising a tumor-directed chimeric antigen receptor and/or tumor-directed chimeric receptor to a subject with cancer.

Cancers derived from B-cell lineages are a worldwide healthcare burden. More than 500,000 new cases of non-Hodgkin lymphoma (NHL) (median age 69 years) and 50,000 new cases of acute lymphoblastic leukemia (ALL) (median age 16 years) are expected in the world each year (seer.cancer.gov, Smith Br J Cancer. 2015; 112(9):1575-84, Solomon, paper presented at: 11^{th} International Conference on Hematology & Hematological Oncology; Nov. 8-9, 2017). Despite progress in treatment, many patients diagnosed with these heterogeneous groups of cancers still succumb to their diseases. Approximately 30% to 50% of newly diagnosed patients with aggressive large cell lymphomas are not cured by first line treatment (Gisselbrecht J Clin Oncol. 2010 Sep. 20; 28(27):4184-90; Kenkre Curr Oncol Rep. 2008:10:393-403; Sehn Blood 2006; 109(5):1857-1861; Sinha Expert Opin Investig Drugs 2011 May 20(5):669-80). Similarly, more than half of adults with ALL will ultimately relapse (Malard 2020).

In some embodiments, the cancer is a hematologic malignancy. In some embodiments, the cancer is a leukemia or a lymphoma. In some embodiments, the lymphoma is a double hit/expressor lymphoma. In some embodiments, the lymphoma is a triple hit/expressor lymphoma. In some embodiments, the cancer comprises Richter's transformation.

Various embodiments provided for herein include treatment or prevention of various malignancies, such as non-Hodgkin lymphoma, B-cell lymphoma, diffuse large B-cell lymphoma, follicular lymphoma, chronic lymphocytic leukemia (CLL), chronic myelogenous leukemia (CML), mantle cell lymphoma, marginal zone lymphomas, Burkitt lymphoma, lymphoplasmacytic lymphoma, hairy cell leukemia, primary central nervous system lymphoma, primary intraocular lymphoma. In some embodiments, the cancer is non-Hodgkin lymphoma. In some embodiments, the cancer is B-cell lymphoma. In some embodiments, the cancer is diffuse large B-cell lymphoma. In some embodiments, the cancer is follicular lymphoma. In some embodiments, the cancer is chronic lymphocytic leukemia. In some embodiments, the cancer is chronic myelogenous leukemia. In some embodiments, the cancer is mantle cell lymphoma. In some embodiments, the cancer is marginal zone lymphoma. Additional types of cancer include, but are not limited to, Hodgkin lymphoma, acute myeloid leukemia (AML), acute lymphoblastic leukemia (ALL), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, cervical cancer, colon cancer, chronic myeloproliferative disorders, ductal carcinoma, endometrial cancer, esophageal cancer, gastric cancer, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, cells of the cancer do not express CD58 or express a mutated form of CD58. In some embodiments, cells of the cancer do not express CD58. In some embodiments, cells of the cancer express a mutated form of CD58. In some embodiments, the mutated form of CD58 comprises a loss of function mutation. In some embodiments, the mutation in CD58 is a loss of function mutation.

In some embodiments, the cancer is a B cell-derived NHL, such as an aggressive large B cell lymphoma (LBCL). In some embodiments, the LBCL is diffuse large B cell lymphoma (DLBCL) not otherwise specified; high grade B cell lymphoma; DLBCL derived from follicular lymphoma (FL) (FL grade 3b); DLBCL derived from Richter's transformation to DLBCL from chronic lymphocytic leukemia (CLL); primary mediastinal LBCL; and DLBCL derived from Waldenstrom macroglobulinemia (WM). In some embodiments, the cancer is a NHL. In some embodiments, the cancer is a LBCL. In some embodiments, the cancer is an aggressive LBCL. In some embodiments, the cancer is DLBCL. In some embodiments, the cancer is FL grade 3b.

In some embodiments, the cancer is an indolent lymphoma (IL). In some embodiments, the IL is a low-grade FL (FL grades 1, 2, and 3a), MCL, or MZL. In some embodiments, the IL is a low-grade FL (FL grades 1, 2, and 3a). In some embodiments, the IL is MCL. In In some embodiments, the IL is MZL. In some embodiments, the cancer is a low grade FL (FL grades 1, 2, and 3a). In some embodiments, the cancer is FL grade 1. In some embodiments, the cancer is FL grade 2. In some embodiments, the cancer is FL grade 3a. In some embodiments, the cancer is MCL. In some embodiments, the cancer is MZL.

In some embodiments, the cancer is CLL or SLL. In some embodiments, the cancer is CLL. In some embodiments, the cancer is SLL. In some embodiments, the cancer is B-ALL.

In some embodiments, the cancer is relapsed/refractory (R/R). In some embodiments, the cancer is R/R NHL. In some embodiments, the cancer is R/R LBCL. In some embodiments, the cancer is R/R CLL. In some embodiments, the cancer is R/R SLL. In some embodiments, the cancer is R/R B-ALL.

In some embodiments, cells of the cancer express CD19. In some embodiments, cells of the cancer express CD19 at the time of administration of a dose (e.g., the first dose) of genetically engineered NK cells. Expression of CD19 can be determined by any methods known in the art, including by flow cytometry.

In some embodiments, cells of the cancer express CD20. In some embodiments, cells of the cancer express CD20 at the time of administration of a dose (e.g., the first dose) of genetically engineered NK cells. Expression of CD20 can be determined by any methods known in the art, including by flow cytometry.

In some embodiments, the cancer has been previously treated with CAR T cells (CAR T exposed). In some embodiments, the cancer is relapsed/refractory to CAR T cells. In some embodiments, the CAR T cells are anti-CD19 CAR T cells. In some embodiments, the CAR T cells are autologous CAR T cells. In some embodiments, the CAR T cells are autologous anti-CD19 CAR T cells. Thus, in some embodiments, the cancer has been previously treated with autologous anti-CD19 CAR T cells. In some embodiments, the cancer is R/R to autologous anti-CD19 CAR T cells. In some embodiments, the cancer has not been previously treated with CAR T cells (CAR T naive). In some embodiments, the cancer has not been previously treated with anti-CD19 CAR T cells, optionally autologous anti-CD19 CAR T cells. Thus, in some embodiments, the cancer is not R/R to anti-CD19 CAR T cells.

In some embodiments, the cancer is aggressive LBCL that has not been previously treated with CAR T cells (CAR T naive), optionally autologous CAR T cells. In some embodiments, the cancer is aggressive LBCL that has not been previously treated with anti-CD19 CAR T cells (CAR T naive), optionally autologous anti-CD19 CAR T cells. In some embodiments, the cancer is a R/R LBCL that has not been previously treated with CAR T cells (CAR T naive), optionally autologous CAR T cells. In some embodiments, the cancer is a R/R LBCL that has not been previously treated with anti-CD19 CAR T cells (CAR T naive), optionally autologous anti-CD19 CAR T cells. In some embodiments, the cancer is a R/R NHL that has not been previously treated with CAR T cells (CAR T naive), optionally autologous CAR T cells. In some embodiments, the cancer is a R/R NHL that has not been previously treated with anti-CD19 CAR T cells (CAR T naive), optionally autologous anti-CD19 CAR T cells. In some embodiments, the cancer is MCL that has not been previously treated with CAR T cells (CAR T naive), optionally autologous CAR T cells. In some embodiments, the cancer is MCL that has not been previously treated with anti-CD19 CAR T cells (CAR T naive), optionally autologous anti-CD19 CAR T cells. In some embodiments, the cancer is an IL that has not been previously treated with CAR T cells (CAR T naive), optionally autologous CAR T cells. In some embodiments, the cancer is an IL that has not been previously treated with anti-CD19 CAR T cells (CAR T naive), optionally autologous anti-CD19 CAR T cells. In some embodiments, the cancer is B-ALL that has not been previously treated with CAR T cells (CAR T naive), optionally autologous CAR T cells. In some embodiments, the cancer is B-ALL that has not been previously treated with anti-CD19 CAR T cells (CAR T naive), optionally autologous anti-CD19 CAR T cells.

In some embodiments, the cancer is aggressive LBCL that has been previously treated with CAR T cells (CAR T exposed), optionally autologous CAR T cells. In some embodiments, the cancer is aggressive LBCL that has been previously treated with anti-CD19 CAR T cells (CAR T exposed), optionally autologous anti-CD19 CAR T cells. In some embodiments, the cancer is a R/R LBCL that has been previously treated with CAR T cells (CAR T exposed), optionally autologous CAR T cells. In some embodiments, the cancer is a R/R LBCL that has been previously treated with anti-CD19 CAR T cells (CAR T exposed), optionally autologous anti-CD19 CAR T cells. In some embodiments, the cancer is a R/R NHL that has been previously treated with CAR T cells (CAR T exposed), optionally autologous CAR T cells. In some embodiments, the cancer is a R/R NHL that has been previously treated with anti-CD19 CAR T cells (CAR T exposed), optionally autologous anti-CD19 CAR T cells.

In some embodiments, the cancer is MCL that has been previously treated with CAR T cells (CAR T exposed), optionally autologous CAR T cells. In some embodiments, the cancer is MCL that has been previously treated with anti-CD19 CAR T cells (CAR T exposed), optionally autologous anti-CD19 CAR T cells. In some embodiments, the cancer is an IL that has been previously treated with CAR T cells (CAR T exposed), optionally autologous CAR T cells. In some embodiments, the cancer is an IL that has been previously treated with anti-CD19 CAR T cells (CAR T exposed), optionally autologous anti-CD19 CAR T cells. In some embodiments, the cancer is B-ALL that has been previously treated with CAR T cells (CAR T exposed), optionally autologous CAR T cells. In some embodiments, the cancer is B-ALL that has been previously treated with anti-CD19 CAR T cells (CAR T exposed), optionally autologous anti-CD19 CAR T cells.

In some embodiments, the cancer is a LBCL (e.g., DLBCL) that has been previously treated with an anti-CD20 monoclonal antibody and a cytotoxic chemotherapy (e.g., anthracycline). In some embodiments, the cancer is a LBCL (e.g., DLBCL) that has been previously treated with an anti-CD20 monoclonal antibody and anthracycline. In some embodiments, the cancer is an IL that has been previously treated with an anti-CD20 monoclonal antibody and a cytotoxic chemotherapy (e.g., anthracycline). In some embodiments, the cancer is an IL that has been previously treated with an anti-CD20 monoclonal antibody and anthracycline.

In some embodiments, the cancer is a MCL, CLL, SLL, or WM that has been previously treated with an inhibitor of Bruton's tyrosine kinase (BTKi) (e.g., ibrutinib). In some embodiments, the cancer is a MCL that has been previously treated with a BTKi (e.g., ibrutinib). In some embodiments, the cancer is a MCL that has been previously treated with a BTKi (e.g., ibrutinib) and anti-CD19 CAR T cells. In some embodiments, the cancer is a CLL that has been previously treated with a BTKi (e.g., ibrutinib). In some embodiments, the cancer is a SLL that has been previously treated with a BTKi (e.g., ibrutinib). In some embodiments, the cancer is a WM that has been previously treated with a BTKi (e.g., ibrutinib).

In some embodiments, the cancer is a CLL or SLL that has been previously treated with a Bcl-2 inhibitor (e.g., venetoclax). In some embodiments, the cancer is a CLL that has been previously treated with a Bcl-2 inhibitor (e.g., venetoclax). In some embodiments, the cancer is a SLL that has been previously treated with a Bcl-2 inhibitor (e.g., venetoclax). In some embodiments, the cancer is a CLL or SLL that has been previously treated with a Bcl-2 inhibitor (e.g., venetoclax) and a BTKi (e.g., ibrutinib). In some embodiments, the cancer is a CLL that has been previously treated with a Bcl-2 inhibitor (e.g., venetoclax) and a BTKi (e.g., ibrutinib). In some embodiments, the cancer is a SLL that has been previously treated with a Bcl-2 inhibitor (e.g., venetoclax) and a BTKi (e.g., ibrutinib).

### Cancer Targets

Some embodiments of the compositions and methods described herein relate to immune cells comprising a chimeric receptor that targets (e.g., binds a cancer antigen, such as CD19, MICA, MICB, ULBP1, ULBP2, ULBP3, ULBP4, ULBP5, and/or ULBP6. In some embodiments, the chimeric receptor targets (e.g., binds) CD19, such as human CD19. Additional non-limiting examples of target antigens include: CD70, CD5; CD123; CD22; CD30; CD171; CS1 (also referred to as CD2 subset 1, CRACC, SLAMF7, CD319, and 19A24); TNF receptor family member B cell maturation (BCMA); CD38; DLL3; G protein coupled receptor class C group 5, member D (GPRC5D); epidermal growth factor receptor (EGFR) CD138; prostate-specific membrane antigen (PSMA); Fms Like Tyrosine Kinase 3 (FLT3); KREMEN2 (Kringle Containing Transmembrane Protein 2), ALPPL2, Claudin 4, Claudin 6, C-type lectin-like molecule-1 (CLL-1 or CLECLI); CD33; epidermal growth factor receptor variant III (EGFRviii); ganglioside G2 (GD2); ganglioside GD3 (aNeu5Ac(2-8)aNeu5Ac(2-3)bDGalp(I-4)bDGlcp(I-I)Cer)); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-IIRa); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (Fra or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(I-4)bDGlcp(I-I)Cer); transglutaminase 5 (TGS5); high molecular weight-melanoma associated antigen (HMWMAA); o-acetyl-GD2 ganglioside (OacGD2); tumor endothelial marker 1 (TEM1/CD248); tumor endothelial marker 7-related (TEM7R); claudin 6 (CLDN6); thyroid stimulating hormone receptor (TSHR); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCRI); adrenoceptor beta 3 (ADRB3); pannexin 3 (PANX3); G protein-coupled receptor 20 (GPR20); lymphocyte antigen 6 complex, locus K 9 (LY6K); Olfactory receptor 51 E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; 56urviving; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase; reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 IB 1 (CYPIB 1); CCCTC-Binding Factor (Zinc Finger Protein)-Like (BORIS or Brother of the Regulator oflmprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Gly cation Endproducts (RAGE-1); renal ubiquitous 1 (RUI1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECi2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLI), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GMI, PTK7, gpNMB, CDH1-CD324, DLL3, CD276/B7H3, III IRa, IL13Ra2, CD179b-IGLII, TCRgamma-delta, NKG2D, CD32 (FCGR2A), Tn ag, Timl-/HVCR1, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLVI-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsgl), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, Claudinl 8.2 (CLD18A2 or CLDN18A.2)), P-glycoprotein, STEAPI, Livl, Nectin-4, Cripto, gpA33, BST1/CD157, low conductance chloride channel, and the antigen recognized by TNT antibody.

### Non-Limiting Embodiments

Among the embodiments provided herein are:
1. A dosing regimen for cancer immunotherapy, comprising:
   at least a first dosing cycle, wherein the first dosing cycle comprises a first dose of genetically engineered natural killer (NK) cells, a second dose of genetically engineered NK cells, and a third dose of genetically engineered NK cells,
   wherein the first dose is administered to a subject in need of cancer immunotherapy at a first time point,
   wherein the second dose is administered to the subject between 5-10 days after the first time point,
   wherein the third dose is administered to the subject between 5-10 days after the second dose;
   wherein each of the first, second and third doses comprise at least about 1.5×10⁹ NK cells,
   wherein at least a portion of the genetically engineered NK cells is engineered to express a chimeric antigen receptor (CAR) that is directed against a CD19 tumor marker,
   wherein the first dosing cycle is initiated after the subject has undergone a lymphodepletion process in order to reduce native immune cell numbers.
2. The dosing regimen of Embodiment 1, wherein the first dosing cycle is followed by one or more additional dosing cycle.
3. The dosing regimen of Embodiment 1 or Embodiment 2, wherein, if the subject exhibits a clinical response, optionally a complete response (CR), following the first dosing cycle, the dosing regimen comprises an additional dosing cycle.
4. The dosing regimen of any one of Embodiments 1 to 3, wherein, if the subject exhibits a clinical response following a dosing cycle and subsequently exhibits disease progression, the dosing regimen comprises an additional dosing cycle.
5. The dosing regimen of any one of Embodiments 1 to 4, wherein the dosing regimen comprises between one dosing cycle and five dosing cycles.
6. The dosing regimen of any one of Embodiments 1 to 5, wherein the subject underdoes a lymphodepletion process prior to each dosing cycle.
7. The dosing regimen of any one of Embodiments 1 to 6, wherein each dosing cycle is between about 14 days and about 35 days.
8. The dosing regimen of any one of Embodiments 1 to 7, wherein each dosing cycle is about 21 days.
9. The dosing regimen of any one of Embodiments 1 to 7, wherein each dosing cycle is about 28 days.
10. The dosing regimen of any one of Embodiments 1 to 9, wherein the lymphodepletion process comprises at least two doses of cyclophosphamide and at least two doses of fludarabine.
11. The dosing regimen of Embodiment 10, wherein the lymphodepletion process comprises three doses of cyclophosphamide and three doses of fludarabine, wherein the first of the doses of cyclophosphamide and fludarabine are administered 5 days prior to the initiation of the first dosing cycle, wherein the second of the doses of cyclophosphamide and fludarabine are administered 4 days prior to the initiation of the first dosing cycle, and wherein the third of the doses of cyclophosphamide and fludarabine are administered 3 days prior to the initiation of the first dosing cycle.
12. The dosing regimen of Embodiment 10 or Embodiment 11, wherein about two days are allowed to lapse between the third of the doses of cyclophosphamide and fludarabine and initiation of the dosing cycle.
13. The dosing regimen of any one of Embodiments 10 to 12, wherein the cyclophosphamide is administered in an amount between about 100 and 600 mg/m² and the fludarabine is administered in an amount between about 10 and 60 mg/m².
14. The dosing regimen of any one of Embodiments 10 to 13, wherein the cyclophosphamide is administered in an amount between about 200 and 600 mg/m² and the fludarabine is administered in an amount between about 20 and 40 mg/m².
15. The dosing regimen of any one of Embodiments 10 to 14, wherein the cyclophosphamide is administered in an amount of about 500 mg/m² and the fludarabine is administered in an amount of about 30 mg/m².
16. The dosing regimen of any one of Embodiments 1 to 15, further comprising administration of a therapeutic agent that targets CD20.
17. The dosing regimen of any one of Embodiments 1 to 15, wherein the subject is administered a therapeutic agent that targets CD20.
18. The dosing regimen of Embodiment 16 or 17, wherein the therapeutic agent is an anti-CD20 monoclonal antibody.
19. The dosing regimen of Embodiment 18, wherein the anti-CD20 antibody is rituximab.
20. The dosing regimen of any one of Embodiments 16 to 19, wherein the therapeutic agent is administered in an amount between about 150 mg/m² and about 500 mg/m².
21. The dosing regimen of any one of Embodiments 16 to 20, wherein the therapeutic agent is administered in an amount of about 375 mg/m².
22. The dosing regimen of any one of Embodiments 16 to 21, wherein the therapeutic agent is administered to the subject at least one time and the at least one time is at least 2 days prior to administration of the first dose of the dosing cycle.
23. The dosing regimen according to any one of Embodiments 16 to 22, wherein the therapeutic agent is administered to the subject one time 3 days prior to administration of the first dose of the dosing cycle.
24. The dosing regimen of any one of Embodiments 1 to 23, wherein the first dose of genetically engineered NK cells is administered to the subject prior to the subject's native immune cell population recovering from the lymphodepletion process, optionally wherein the first and second doses of genetically engineered NK cells are administered to the subject prior to the subject's native immune cell population recovering from the lymphodepletion process.
25. The dosing regimen of any one of Embodiments 1 to 24, wherein the first dose of genetically engineered NK cells is administered to the subject about 2 to 5 days after completion of the lymphodepletion process.
26. The dosing regimen of any one of Embodiments 1 to 25, wherein the cancer is a blood cancer.
27. The dosing regimen of any one of Embodiments 1 to 26, wherein the cancer is a leukemia or a lymphoma.
28. The dosing regimen of any one of embodiments 1 to 27, wherein the cancer is a B cell cancer.
29. The dosing regimen of any one of embodiments 1 to 28, wherein the cancer is a Non-Hodgkin lymphoma (NHL).
30. The dosing regimen of any one of Embodiments 1 to 29, wherein the cancer is a large B-cell lymphoma (LBCL), optionally an aggressive LBCL.
31. The dosing regimen of any one of Embodiments 1 to 30, wherein the cancer is diffuse large B-cell lymphoma (DLBCL), follicular lymphoma (FL), marginal zone lymphoma (MZL), mantle cell lymphoma (MCL), Waldenstr6 m macroglobulinemia (WM), or B-cell acute lymphoblastic leukemia (B-ALL).
32. The dosing regimen of any one of Embodiments 1 to 28, wherein the cancer is a chronic lymphocytic leukemia (CLL) or a small lymphocytic lymphoma (SLL).
33. The dosing regimen of any one of Embodiments 1 to 32, wherein the cancer is a relapsed/refractory (R/R) cancer.
34. The dosing regimen of any one of Embodiments 1 to 33, wherein the subject has less than or equal to 5% peripheral blasts.
35. The dosing regimen of any one of Embodiments 1 to 34, wherein the subject has received at least 1 but not more than 7 lines of previous therapy, optionally wherein the subject has received at least 1 but not more than 4 lines of previous therapy.
36. The dosing regimen of any one of Embodiments 1 to 35, wherein the subject has received at least one line of previous therapy.
37. The dosing regimen of any one of Embodiments 1 to 36, wherein the subject has received at least two lines of previous therapy.
38. The dosing regimen of any one of Embodiments 35 to 37, wherein a line of previous therapy comprises an anti-CD20 monoclonal antibody and a cytotoxic chemotherapy, optionally wherein the cytotoxic therapy is anthracycline.
39. The dosing regimen of any one of Embodiments 35 to 38, wherein a line of previous therapy comprises chimeric antigen receptor-expressing T (CAR T) cells, optionally wherein a line of previous therapy comprises autologous anti-CD19 CAR T cells.
40. The dosing regimen of any one of Embodiments 35 to 38, wherein a line of previous therapy does not comprise CAR T cells, optionally wherein a line of previous therapy does not comprise autologous anti-CD19 CAR T cells.
41. The dosing regimen of any one of Embodiments 35 to 40, wherein a line of previous therapy comprises an inhibitor of Bruton's tyrosine kinase (BTKi), optionally wherein the BTKi is ibrutinib.
42. The dosing regimen of any one of Embodiments 35 to 41, wherein a line of previous therapy comprises an inhibitor of Bcl-2, optionally wherein the Bcl-2 inhibitor is venetoclax.
43. The dosing regimen of any one of Embodiments 1 to 42, wherein the second and third doses of genetically engineered NK cells are administered to the subject within about 21 days of the first time point.
44. The dosing regimen of any one of Embodiments 1 to 43, wherein the second and third doses of genetically engineered NK cells are administered to the subject within about 14 days after the first time point.
45. The dosing regimen of any one of Embodiments 1 to 44, wherein the CAR comprises:
   (a) an antigen-binding moiety that targets CD19;
   (b) a transmembrane domain; and
   (c) an intracellular signaling domain comprising an OX40 domain and a CD3zeta domain.
46. The dosing regimen of Embodiment 45, wherein the antigen-binding moiety comprises a heavy chain variable region (VH) and a light chain variable region (VL), wherein:
   the VH comprises a CDR-1, a CDR-2, and a CDR-3 comprising the amino acid sequences set forth in SEQ ID NOS: 45, 46, and 47, respectively; and the VL comprises a CDR-1, a CDR-2, and a CDR-3 comprising the amino acid sequences set forth in SEQ ID NOS: 48, 49, and 16, respectively;
   the VH comprises a CDR-1, a CDR-2, and a CDR-3 comprising the amino acid sequences set forth in SEQ ID NOS: 50, 23, and 24, respectively; and the VL comprises a CDR-1, a CDR-2, and a CDR-3 comprising the amino acid sequences set forth in SEQ ID NOS: 14, 15, and 16, respectively;
   the VH comprises the amino acid sequence set forth in SEQ ID NO: 21 and/or the VL comprises the amino acid sequence set forth in SEQ ID NO:13; and/or
   the antigen-binding domain is an scFv comprising the amino acid sequence of SEQ ID NO:6.
47. The dosing regimen of any one of Embodiments 1 to 46, wherein the genetically engineered NK cells are also engineered to express membrane-bound interleukin 15 (mblL15).
48. The dosing regimen of Embodiment 47, wherein the mblL15 has at least 95% sequence identity to SEQ ID NO: 44.
49. The dosing regimen of any one of Embodiments 1 to 48, wherein the dosing regimen does not result in cytokine release syndrome, immune effector cell-associated neurotoxicity syndrome (ICANS)/neurotoxicity, and/or graft versus host disease.
50. The dosing regimen of any one of Embodiments 1 to 49, wherein the engineered NK cells are allogeneic with respect to the subject.
51. The dosing regimen of any one of Embodiments 1 to 50, wherein the subject has a 158V/158V CD16 genotype.
52. The dosing regimen of any one of Embodiments 1 to 51, wherein the subject has a 158F/1 58F CD16 genotype.
53. A dosing regimen for cancer immunotherapy, comprising:
   at least a first dosing cycle, wherein the first dosing cycle comprises a first dose of genetically engineered natural killer (NK) cells, a second dose of genetically engineered NK cells, and a third dose of genetically engineered NK cells,
   wherein the first dose is administered to a subject in need of cancer immunotherapy at a first time point,
   wherein the second dose is administered to the subject between 5-10 days after the first time point,
   wherein the third dose is administered to the subject between 5-10 days after the second dose; wherein each of the first, second and third doses comprise at least 1.0×10⁹ NK cells,
   wherein at least a portion of the engineered NK cells is engineered to express a chimeric antigen receptor (CAR) that is directed against a CD19 tumor marker,
   wherein the first dosing cycle is initiated after the subject has undergone a lymphodepletion process comprising at least two doses of cyclophosphamide and fludarabine, and
   wherein an anti-CD20 antibody is administered during the lymphodepletion process.
54. The dosing regimen of embodiment 53, the first dosing cycle is followed by one or more additional dosing cycle.
55. The dosing regimen of Embodiment 53 or Embodiment 54, wherein, if the subject exhibits a clinical response, optionally a complete response (CR), following the first dosing cycle, the dosing regimen comprises an additional dosing cycle.
56. The dosing regimen of any one of Embodiments 53 to 55, wherein, if the subject exhibits a clinical response following a dosing cycle and subsequently exhibits disease progression, the dosing regimen comprises an additional dosing cycle.
57. The dosing regimen of any one of Embodiments 53 to 56, wherein the dosing regimen comprises between one dosing cycle and five dosing cycles.
58. The dosing regimen of any one of Embodiments 53 to 57, wherein the subject underdoes a lymphodepletion process prior to each dosing cycle.
59. The dosing regimen of any one of Embodiments 1 to 58, wherein each of the three doses of NK cells comprises about 1.5×10⁹ NK cells.
60. The dosing regimen of any one of Embodiments 53 to 59, wherein the lymphodepletion process comprises three doses of cyclophosphamide and three doses of fludarabine,
   wherein the first of the doses of cyclophosphamide and fludarabine are administered 5 days prior to the initiation of the dosing cycle,
   wherein the second of the doses of cyclophosphamide and fludarabine are administered 4 days prior to the initiation of the dosing cycle,
   wherein the third of the doses of cyclophosphamide and fludarabine are administered 3 days prior to the initiation of the dosing cycle, and
   wherein the anti-CD20 antibody is administered on the same day as the third dose of cyclophosphamide and fludarabine.
61. The dosing regimen of any one of Embodiments 53 to 60, wherein the cyclophosphamide is administered in an amount between about 300 and 600 mg/m², wherein the fludarabine is administered in an amount between about 20 and 40 mg/m², wherein the anti-CD20 antibody is administered in an amount between about 350 mg/m² and about 425 mg/m², and wherein each of the three doses of NK cells comprise at least 1.5×109 NK cells.
62. The dosing regimen of any one of Embodiments 53 to 61, wherein the anti-CD20 antibody comprises rituximab or obinutuzumab.
63. The dosing regimen of any one of Embodiments 1 to 62, wherein cells of the cancer do not express CD58 or express a mutated form of CD58.
64. The dosing regimen of any one of Embodiments 1 to 63, wherein, prior to administration of the first dosing cycle to the subject, cells of the cancer are determined not to express CD58 or to express a mutated form of CD58.
65. The dosing regimen of any one of Embodiments 1 to 64, wherein, prior to administration of the first dosing cycle to the subject, the subject has been selected for treatment with the dosing regimen based on cells of the cancer exhibiting a loss or mutation of CD58.
66. The dosing regimen of any one of Embodiments 1 to 65, wherein one dose of each dosing cycle is administered to the subject on an outpatient basis, optionally wherein each dose of each dosing cycle is administered to the subject on an outpatient basis.
67. The dosing regimen of any one of Embodiments 1 to 66, wherein, among subjects treated according to the dosing regimen, the overall response rate (ORR) is at least about 50%, at least about 60%, at least about 70%, or at least about 80%.
68. The dosing regimen of any one of Embodiments 1 to 67, wherein at least about 50%, at least about 60%, at least about 70%, or at least about 80% of subjects treated according to the dosing regimen exhibit a complete response (CR).
69. A method for the treatment of cancer, comprising administering, to a subject having a cancer, genetically engineered natural killer (NK) cells expressing a chimeric antigen receptor (CAR) that is directed against an antigen associated with or expressed by cells of the cancer, wherein:
   the cancer does not express CD58 or expresses a mutated form of CD58; and
   the subject is relapsed and/or refractory to genetically engineered T cells expressing a CAR directed against the antigen.
70. The method of embodiment 69, wherein the antigen is CD19.
71 A method for the treatment of cancer, comprising administered, to a subject having a cancer, genetically engineered natural killer (NK) cells expressing a chimeric antigen receptor (CAR) that is directed against CD19, wherein the cancer does not express CD58 or expresses a mutated form of CD58.
72. The method of any one of embodiments 69 to 71, further comprising:
   determining that the cancer that does not express CD58 or expresses a mutated form of CD58;
   identifying the subject as having a cancer that does not express CD58 or expresses a mutated form of CD58; and/or
   selecting the identified subject for treatment with the genetically engineered NK cells.
73. The method of embodiment 71 or embodiment 72, wherein the subject was previously treated with genetically engineered T cells expressing a CAR that is directed against CD19 for the cancer, optionally wherein the subject is relapsed and/or refractory to the genetically engineered T cells.
74. The method of any one of Embodiments 69 to 73, wherein the genetically engineered NK cells are administered in a dosing regimen comprising at least a first dosing cycle, wherein the first dosing cycle comprises a first dose of the genetically engineered NK cells, a second dose of the genetically engineered NK cells, and a third dose of the genetically engineered NK cells,
   wherein the first dose is administered to the subject at a first time point,
   wherein the second dose is administered to the subject between 5-10 days after the first time point,
   wherein the third dose is administered to the subject between 5-10 days after the second dose; and
   wherein each of the first, second and third doses comprises at least about 1.5×10⁹ NK cells, optionally at least about 1.5×10⁹ CAR-expressing NK cells.
75. A method for the treatment of cancer, comprising:
   (a) selecting a subject for the treatment of cancer if cells of the cancer do not express CD58 or express a mutated form of CD58;
   (b) administering to the selected subject at least a first dosing cycle, wherein the first dosing cycle comprises a first dose of genetically engineered natural killer (NK) cells, a second dose of genetically engineered NK cells, and a third dose of genetically engineered NK cells,
   wherein the first dose is administered to a subject in need of cancer immunotherapy at a first time point,
   wherein the second dose is administered to the subject between 5-10 days after the first time point,
   wherein the third dose is administered to the subject between 5-10 days after the second dose;
   wherein each of the first, second and third doses comprise at least about 1.5×10⁹ NK cells, and
   wherein at least a portion of the genetically engineered NK cells is engineered to express a chimeric antigen receptor (CAR) that is directed against a CD19 tumor marker.
76. The method of embodiment 74 or embodiment 75, wherein the first dosing cycle is initiated after the subject has undergone a lymphodepletion regimen to reduce native immune cell numbers.
77. A method for the treatment of cancer, comprising:
   administering to a subject having cancer a lymphodepletion regimen comprising at least two doses of cyclophosphamide and at least two doses of fludarabine;
   administering to the subject a dosing cycle comprising at least a first, a second, and a third dose of genetically engineered NK cells,
   wherein the first dose of genetically engineered NK cells is administered to the subject after the last dose of fludarabine,
   wherein the second dose of genetically engineered NK cells is administered to the subject between 6-8 days after the first dose of genetically engineered NK cells,
   wherein the third dose of genetically engineered NK cells is administered to the subject between 6-8 days after the second dose of genetically engineered NK cells;
   wherein each of the first, second and third doses of genetically engineered NK cells comprises about 1.5×10⁹ NK cells, and
   wherein the genetically engineered NK cells are allogeneic with respect to the subject and are engineered to express a chimeric antigen receptor (CAR) that binds CD19.
78. A method for the treatment of cancer, comprising:
   administering to a subject having cancer a lymphodepletion regimen comprising at least two doses of cyclophosphamide and at least two doses of fludarabine;
   administering to the subject an agent that binds CD20;
   administering to the subject a dosing cycle comprising at least a first, a second, and a third dose of genetically engineered NK cells,
   wherein the first dose of genetically engineered NK cells is administered to the subject after the last dose of fludarabine,
   wherein the second dose of genetically engineered NK cells is administered to the subject between 6-8 days after the first dose of genetically engineered NK cells,
   wherein the third dose of genetically engineered NK cells is administered to the subject between 6-8 days after the second dose of genetically engineered NK cells;
   wherein each of the first, second and third doses of genetically engineered NK cells comprises at least 1.0×10⁹ NK cells, and
   wherein the genetically engineered NK cells are allogeneic with respect to the subject and are engineered to express a chimeric antigen receptor (CAR) that binds CD19.
79. The method of any one of EmbodimentEmbodiments 74, 76, and 78, wherein each of the first, second and third doses comprises at least 1.5×10⁹ NK cells, optionally at least 1.5×10⁹ CAR-expressing NK cells.
80. The method of any one of Embodiments 76 to 79, wherein the lymphodepletion regimen comprises three doses of cyclophosphamide and three doses of fludarabine, wherein the first of the doses of cyclophosphamide and fludarabine are administered 5 days prior to the first dose of genetically engineered NK cells, wherein the second of the doses of cyclophosphamide and fludarabine are administered 4 days prior to the of genetically engineered NK cells, and wherein the third of the doses of cyclophosphamide and fludarabine are administered 3 days prior to the initiation of the dosing cycle.
81. The method of any one of Embodiments 77 to 80, wherein the cyclophosphamide is administered in an amount between about 100 and 600 mg/m² and the fludarabine is administered in an amount between about 20 and 40 mg/m².
82. The method of any one of Embodiments 77 to 81, wherein the cyclophosphamide is administered in an amount of about 500 mg/m² and the fludarabine is administered in an amount of about 30 mg/m².
83. The method of any one of Embodiments 77 to 82, wherein the agent that binds CD20 is an anti-CD20 monoclonal antibody, wherein the anti-CD20 monoclonal antibody is administered three days before the first dose of genetically engineered NK cells is administered to the subject, and wherein the anti-CD20 monoclonal antibody is administered in an amount between about 350 mg/m² and about 425 mg/m².
84. The method of Embodiment 83, wherein the wherein the anti-CD20 monoclonal antibody comprises rituximab or obinutuzumab.
85. The method of any one of Embodiments 69 to 84, wherein the cancer is a blood cancer.
86. The method of any one of Embodiments 69 to 85, wherein the cancer is a leukemia or a lymphoma.
87. The method of any one of Embodiments 69 to 86, wherein the cancer is a B cell cancer.
88. The method of any one of Embodiments 69 to 87, wherein the cancer is a Non-Hodgkin lymphoma (NHL).
89. The method of any one of Embodiments 69 to 88, wherein the cancer is a large B-cell lymphoma (LBCL), optionally an aggressive LBCL.
90. The method of any one of Embodiments 69 to 89, wherein the cancer is diffuse large B-cell lymphoma (DLBCL), follicular lymphoma (FL), marginal zone lymphoma (MZL), mantle cell lymphoma (MCL), Waldenstr6 m macroglobulinemia (WM), or B-cell acute lymphoblastic leukemia (B-ALL).
91. The method of any one of Embodiments 69 to 87, wherein the cancer is a chronic lymphocytic leukemia (CLL) or a small lymphocytic lymphoma (SLL).
92. The method of any one of Embodiments 69 to 91, wherein the cancer is a relapsed/refractory (R/R) cancer.
93. The method of any one of Embodiments 69 to 92, wherein the subject has less than or equal to 5% peripheral blasts.
94. The method of any one of Embodiments 69 to 93, wherein the CAR comprises:
   (a) an antigen-binding moiety that targets CD19;
   (b) a transmembrane domain; and
   (c) an intracellular signaling domain comprising an OX40 domain and a CD3zeta domain.
95. The method of Embodiment 94, wherein the antigen-binding moiety comprises a heavy chain variable region (VH) and a light chain variable region (VL), wherein:
   the VH comprises a CDR-1, a CDR-2, and a CDR-3 comprising the amino acid sequences set forth in SEQ ID NOS: 45, 46, and 47, respectively; and the VL comprises a CDR-1, a CDR-2, and a CDR-3 comprising the amino acid sequences set forth in SEQ ID NOS: 48, 49, and 16, respectively;
   the VH comprises a CDR-1, a CDR-2, and a CDR-3 comprising the amino acid sequences set forth in SEQ ID NOS: 50, 23, and 24, respectively; and the VL comprises a CDR-1, a CDR-2, and a CDR-3 comprising the amino acid sequences set forth in SEQ ID NOS: 14, 15, and 16, respectively;
   the VH comprises the amino acid sequence set forth in SEQ ID NO: 21 and/or the VL comprises the amino acid sequence set forth in SEQ ID NO:13; and/or
   the antigen-binding domain is an scFv comprising the amino acid sequence of SEQ ID NO:6.
96. The method of any one of Embodiments 69 to 95, wherein the genetically engineered NK cells are also engineered to express membrane-bound interleukin 15 (mblL15).
97. The method of embodiment 96, wherein the mblL15 has at least 95% sequence identity to SEQ ID NO: 44.
98. The method of any one of Embodiments 74 to 97, wherein administration of the dosing cycle does not result in cytokine release syndrome, immune effector cell-associated neurotoxicity syndrome (ICANS)/neurotoxicity, and/or graft versus host disease.
99. The method of any one of Embodiments 75 to 98, wherein cells of the cancer do not express CD58 or express a mutated form of CD58.
100. The method of any one of Embodiments 77 to 99, wherein: (a) prior to administration of a first dose of genetically engineered NK cells to the subject, cells of the cancer have been determined not to express CD58 or to express a mutated form of CD58; and/or (b) the method further comprises selecting the subject for treatment based on cells of the cancer exhibiting a loss of or mutation in CD58.
101. The method of any one of Embodiments 74 to 100, wherein one dose of the dosing cycle is administered to the subject on an outpatient basis, optionally wherein each dose of the dosing cycle is administered to the subject on an outpatient basis.
102. Use of a population of engineered NK cells expressing a chimeric antigen receptor that targets CD19 for treating cancer, by administration of a dosing cycle comprising at least a first, a second, and a third dose of said genetically engineered NK cells,
   wherein the first dose of genetically engineered NK cells is administered to the subject after a final dose of a lymphodepletion regimen is administered to the subject, the lymphodepletion regimen comprising at least two doses of cyclophosphamide and at least two doses of fludarabine,
   wherein the second dose of genetically engineered NK cells is administered to the subject between 6-8 days after the first dose of genetically engineered NK cells,
   wherein the third dose of genetically engineered NK cells is administered to the subject between 6-8 days after the second dose of genetically engineered NK cells, and
   wherein each of the first, second and third doses of genetically engineered NK cells comprises about 1.5×10⁹ genetically engineered NK cells.
103. Use of a population of engineered NK cells expressing a chimeric antigen receptor that targets CD19 for treating cancer, by administration of a dosing cycle comprising at least a first, a second, and a third dose of said genetically engineered NK cells,
   wherein the first dose of genetically engineered NK cells is administered to the subject after a final dose of a lymphodepletion regimen is administered to the subject, the lymphodepletion regimen comprising at least two doses of cyclophosphamide and at least two doses of fludarabine,
   wherein the first dose of genetically engineered NK cells is administered to the subject after administration of an agent that binds CD20,
   wherein the second dose of genetically engineered NK cells administered to the subject between 6-8 days after the first dose,
   wherein the third dose of genetically engineered NK cells is administered to the subject between 6-8 days after the second dose, and
   wherein each of the first, second and third doses of genetically engineered NK cells comprises about 1.5×10⁹ genetically engineered NK cells.
104. The use of Embodiment 102 or Embodiment 103, wherein each of the first, second and third doses of genetically engineered NK cells comprises at least 1.5×10⁹ NK cells, optionally at least 1.5×10⁹ CAR-expressing NK cells.
105. The use of any one of Embodiments 102 to 104, wherein the lymphodepletion regimen comprises three doses of cyclophosphamide and three doses of fludarabine, wherein the first of the doses of cyclophosphamide and fludarabine are administered 5 days prior to the first dose of genetically engineered NK cells, wherein the second of the doses of cyclophosphamide and fludarabine are administered 4 days prior to the first dose of genetically engineered NK cells, and wherein the third of the doses of cyclophosphamide and fludarabine are administered 3 days prior to the first dose of genetically engineered NK cells.
106. The use of any one of Embodiments 102 to 105, wherein the agent that binds CD20 is administered 3 days prior to the first dose of genetically engineered NK cells.
107. The use of any one of Embodiments 102 to 106, wherein the cyclophosphamide is administered in an amount between about 100 and 600 mg/m² and the fludarabine is administered in an amount between about 10 and 60 mg/m².
108. The use of any one of Embodiments 102 to 107, wherein the cyclophosphamide is administered in an amount between about 300 and 600 mg/m² and the fludarabine is administered in an amount between about 20 and 40 mg/m².
109. The use of any one of Embodiments 102 to 108, wherein the cyclophosphamide is administered in an amount of about 500 mg/m² and the fludarabine is administered in an amount of about 30 mg/m².
110. The use of any one of Embodiments 102 to 119, wherein the agent that binds CD20 is an anti-CD20 monoclonal antibody selected from rituximab, obinutuzumab, and combinations thereof, and wherein the anti-CD20 monoclonal antibody is administered in an amount between about 350 mg/m² and about 425 mg/m².
111. The use of Embodiment 110, wherein the anti-CD20 monoclonal antibody comprises rituximab, and wherein the rituximab is administered in an amount of about 375 mg/m².
112. The use of any one of Embodiments 102 to 111, wherein the cancer is a blood cancer.
113. The use of any one of Embodiments 102 to 112, wherein the cancer is a B cell cancer.
114. The use of any one of Embodiments 120 to 113, wherein the cancer is a leukemia or a lymphoma.
115. The use of any one of Embodiments 102 to 114, wherein the cancer is a Non-Hodgkin lymphoma (NHL).
116. The use of any one of Embodiments 102 to 115, wherein the cancer is a large B-cell lymphoma (LCBL), optionally an aggressive LBCL.
117. The use of any one of Embodiments 102 to 116, wherein the cancer is diffuse large B-cell lymphoma (DLBCL), follicular lymphoma (FL), marginal zone lymphoma (MZL), mantle cell lymphoma (MCL), Waldenstrom macroglobulinemia (WM), or B-cell acute lymphoblastic leukemia (B-ALL).
118. The use of any one of Embodiments 102 to 114, wherein the cancer is a chronic lymphocytic leukemia (CLL) or a small lymphocytic lymphoma (SLL).
119. The use of any one of Embodiments 102 to 118, wherein the cancer is a relapsed/refractory (R/R) cancer.
120. The use of any one of Embodiments 102 to 119, wherein the subject has less than or equal to 5% peripheral blasts.
121. The use of any one of Embodiments 102 to 120, wherein the CAR comprises:
   (a) an antigen-binding moiety that targets CD19;
   (b) a transmembrane domain; and
   (c) an intracellular signaling domain comprising an OX40 domain and a CD3zeta domain.
122. The use of Embodiment 121, wherein the antigen-binding moiety comprises a heavy chain variable region (VH) and a light chain variable region (VL), wherein:
   the VH comprises a CDR-1, a CDR-2, and a CDR-3 comprising the amino acid sequences set forth in SEQ ID NOS: 45, 46, and 47, respectively; and the VL comprises a CDR-1, a CDR-2, and a CDR-3 comprising the amino acid sequences set forth in SEQ ID NOS: 48, 49, and 16, respectively;
   the VH comprises a CDR-1, a CDR-2, and a CDR-3 comprising the amino acid sequences set forth in SEQ ID NOS: 50, 23, and 24, respectively; and the VL comprises a CDR-1, a CDR-2, and a CDR-3 comprising the amino acid sequences set forth in SEQ ID NOS: 14, 15, and 16, respectively;
   the VH comprises the amino acid sequence set forth in SEQ ID NO: 21 and/or the VL comprises the amino acid sequence set forth in SEQ ID NO:13; and/or
   the antigen-binding domain is an scFv comprising the amino acid sequence of SEQ ID NO:6.
123. The use of any one of Embodiments 102 to 122, wherein the genetically engineered NK cells are also engineered to express membrane-bound interleukin 15 (mblL15).
124. The use of Embodiment 123, wherein the mblL15 has at least 95% sequence identity to SEQ ID NO: 44.
125. The use of any one of Embodiments 102 to 124, wherein the dosing cycle does not result in cytokine release syndrome, immune effector cell-associated neurotoxicity syndrome (ICANS)/neurotoxicity, and/or graft versus host disease.
126. Use of a population of engineered NK cells expressing a chimeric antigen receptor that targets CD19 for treating cancer in a subject, by intravenous administration of a dosing cycle comprising at least three sequential doses of genetically engineered NK cells,
   wherein the first dose of genetically engineered NK cells is administered to the subject at a first time point and comprises at least 1.5×10⁹ engineered NK cells,
   wherein the second dose of genetically engineered NK cells is administered to the subject between 6-8 days after the first dose of genetically engineered NK cells and comprises at least 1.5×10⁹ engineered NK cells,
   wherein the third dose of genetically engineered NK cells is administered to the subject between 6-8 days after the second dose of genetically engineered NK cells and comprises at least 1.5×10⁹ engineered NK cells, and
   wherein the engineered NK cells express a CD19 CAR having at least 95% sequence identity to SEQ ID NO: 43.
127. The use of Embodiment 126, further comprising administering at least one agent that targets CD20 prior to the first dose of genetically engineered NK cells.
128. The use of Embodiment 127, wherein the agent that targets CD20 comprises rituximab.
129. Use of genetically engineered natural killer (NK) cells expressing a chimeric antigen receptor (CAR) that is directed against an antigen associated with or expressed by cells of a cancer for treating a subject having the cancer, wherein:
   the cancer does not express CD58 or expresses a mutated form of CD58; and
   the subject is relapsed and/or refractory to genetically engineered T cells expressing a CAR directed against the antigen.
130. Use of genetically engineered natural killer (NK) cells expressing a chimeric antigen receptor (CAR) that is directed against CD19 for treating a subject having a cancer, wherein the cancer does not express CD58 or expresses a mutated form of CD58.
131. The use of Embodiment 129 or Embodiment 130, wherein the genetically engineered NK cells are for administration in a dosing regimen comprising at least a first dosing cycle, wherein the first dosing cycle comprises a first dose of the genetically engineered NK cells, a second dose of the genetically engineered NK cells, and a third dose of the genetically engineered NK cells,
   wherein the first dose is administered to the subject at a first time point,
   wherein the second dose is administered to the subject between 5-10 days after the first time point,
   wherein the third dose is administered to the subject between 5-10 days after the second dose; and
   wherein each of the first, second and third doses comprises at least about 1.5×10⁹ NK cells, optionally at least about 1.5×10⁹ CAR-expressing NK cells.

### EXAMPLES

The following are non-limiting descriptions of experimental methods and materials that will be used in examples disclosed below.

### Example 1—First Dosing Regimen for NK Cell Immunotherapy

As discussed in more detail herein, certain cancer types express selected markers in an elevated manner. In several embodiments, cytotoxic receptor constructs are generated according to sequences disclosed herein in order to specifically target a given cancer. For example, many cancers express elevated levels of CD19, including Non-Hodgkin's lymphoma. Thus, as discussed in detail above, in several embodiments, CD19-targeting CAR constructs are provided. In several embodiments, the polynucleotides encoding those constructs are engineered to bi-cistronically express mblL15 (e.g., SEQ ID NO: 1 or SEQ ID NO: 53). A dosing regimen was tested to evaluate the efficacy of cells expressing such constructs. The dosing regimen employed NK cells (from healthy donors) engineered to express the CD19 CAR. In several embodiments, the engineered NK cells express the cytotoxic receptor encoded by SEQ ID NO: 3 (including degeneracies or codon-optimized versions of SEQ ID NO: 3). In several embodiments, the engineered NK cells express the cytotoxic receptor comprising the amino acid sequence of SEQ ID NO: 43 (e.g., SEQ ID NO: 4) (and, optionally mblL15 comprising the amino acid sequence of SEQ ID NO: 44 (e.g., SEQ ID NO: 40).

The dosing regimen was designed to evaluate engineered NK cells that are administered three times in a 28-day dosing cycle to treat Non-Hodgkin's lymphoma. All subjects had relapsed/refractory CD19+B-cell malignancies including large B-cell lymphoma (LBCL, including diffuse large B-cell lymphoma (DLBCL) and follicular lymphoma grade 3b (FL3b)), mantle cell lymphoma (MCL), follicular lymphoma (FL), and marginal zone lymphoma (MZL); had received two or more prior lines of therapy; had an ECOG status of 0 or 1; and were CAR T cell therapy naive. The dosing cycle was preceded by a conditioning phase during which subjects underwent lymphodepletion (using cyclophosphamide (300 mg/m²) and fludarabine (30 mg/m²) at day-5, day-4, and day-3). At day 0 the subjects received the first dose of 1×10⁹ CD19 CAR-NK cells. Dose 2 was administered at day 7 and dose 3 was administered at day 14. At day 28, outcome measures were assessed.

Primary endpoints included: (1) incidence, nature, and severity of treatment related adverse events will be evaluated with an adverse event defined as any unfavorable and unintended sign including clinically significant abnormal laboratory findings, symptom or disease measured 30 days after the last dose of the NK cells; and (2) proportion of subjects experiencing dose-limiting toxicities (DLTs) of the NK cells, with DLTs defined as adverse events attributable to treatment that occur during Cycle 1 and meet protocol specified criteria measured 28 days from the first dose of NK cells.

Secondary outcome measures included evaluation of: (1) pharmacokinetic parameters in the context of the immune system, including but not limited to maximum concentration (Cₘₐₓ), time to reach maximum concentration (Tₘₐₓ), area under the concentration-time curve (AUC), half-life (t_{1/2}), and duration of persistence of the CD19 CAR-NK cells in the peripheral blood and other target tissues such as bone marrow; (2) humoral and cellular immunogenicity against the CD19 CAR-NK cells; (3) changes in serum cytokine levels such as interferon-gamma (IFN-γ) and other host responses to CD19 CAR-NK cells in peripheral; (4) best overall response rates in dose finding and safety lead-in cohorts; and/or (5) other antitumor measurements, which may include duration of response (DOR), time-to-first response, time-to-best response, bridge-to-transplant rate, event-free survival (EFS), progression free survival (PFS), and overall survival (OS) using standard disease specific response assessment criteria.

After assessing the primary endpoints, it was determined the CD19 CAR NKs were well tolerated at the 1×10⁹ dose (3 doses over 28 days). No DLTs were identified. Myelosuppression (consistent with lymphodepletion) was the most common higher-grade toxicity. One subject (of six subjects) experienced a grade 1 infusion reaction with a transient fever. No CAR T-like cytotoxicities were detected.

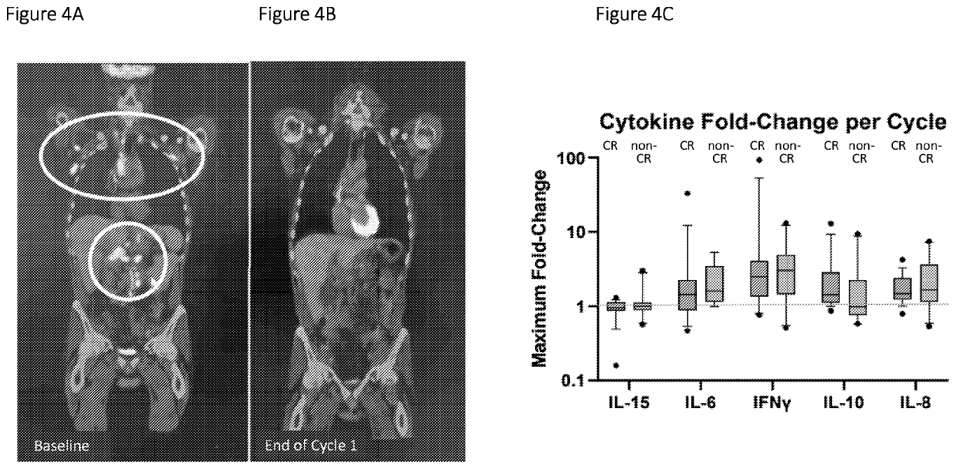

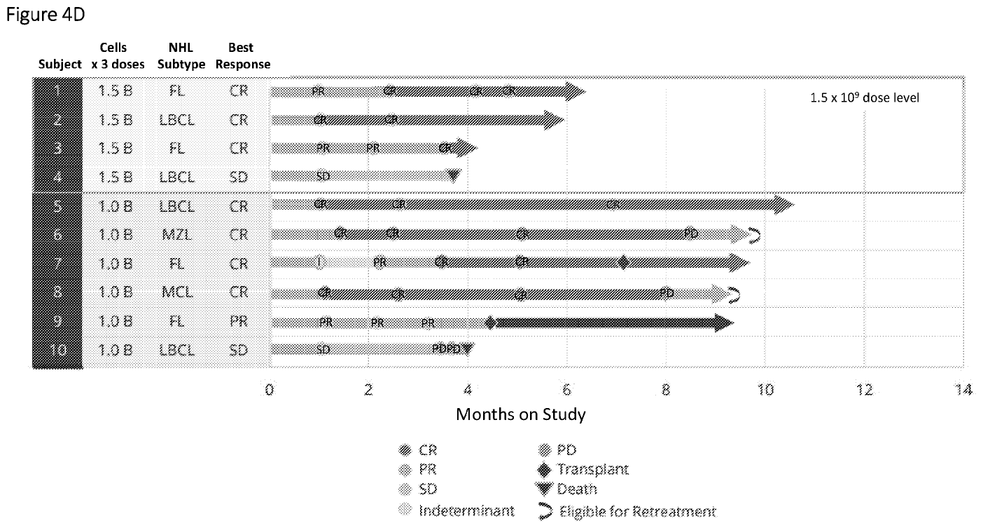

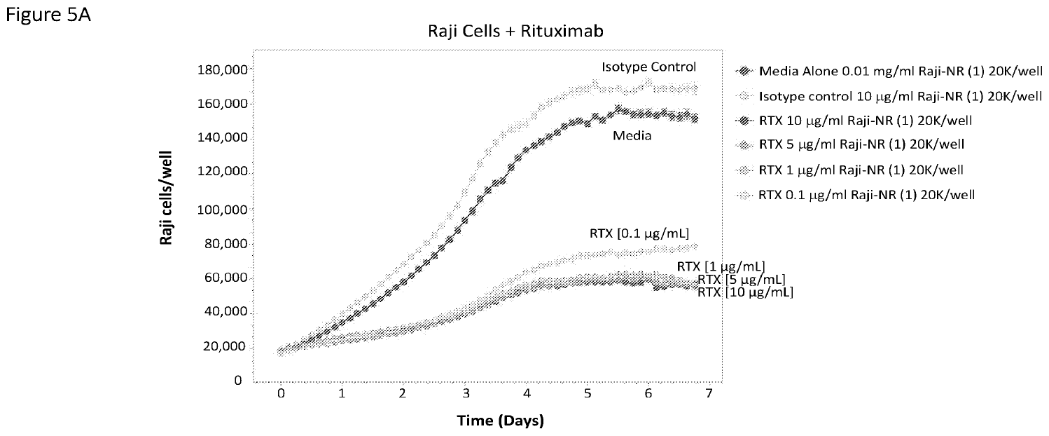

FIG. 3 shows data summarizing the overall response rate in 6 NHL patients treated according to this dosing regimen. When considered as a whole, over 80% of NHL patients demonstrated complete response (CR) or partial response (PR). Three of six subjects showed complete response when all NHL subtypes were considered, with 100% of the subjects having marginal zone lymphoma (MZL) and mantle cell lymphoma (MCL) showing complete response. FIGS. 4A-4B show data from a 53-year-old male subject with extensive DLBCL who had relapsed after R-EPOCH (Rituximab, Etoposide phosphate, Prednisone, vincristine sulfate (Oncovin®), Cyclophosphamide, and doxorubicin hydrochloride (Hydroxydaunorubicin)) and R-ICE (Rituximab, Ifosfamide, Carboplatin, and Etoposide phosphate) treatments. These data demonstrated that engineered immune cells (e.g., NK cells) expressing a CD19-directed CAR offer effective treatment of NHL, including various subtypes of NHL.

An additional 4 subjects were treated according to the same dosing regimen, except that each of the three doses contained 1.5×10⁹ CD19 CAR-NK cells. The 1.5×10⁹ cell dose was well tolerated, with no immune effector cell-associated neurotoxicity syndrome (ICANS)/neurotoxicity, GvHD, or DLTs observed. No treatment-related adverse events leading to discontinuation of treatment were observed. One subject had a Grade≥3 infection. As with the 1×10⁹ dose, myelosuppression consistent with lymphodepletion was the most common Grade≥3 toxicity and was manageable. A minority of patients experienced transient and manageable infusion-related effects (fever within 8 hours that resolved within 24 hours), but no association between symptoms and response was observed. No>Grade 3 CRS was observed. Cytokine elevation was generally modest across all subjects, and no observation was observed between elevated serum cytokines and clinical response (FIG. 4C; CR on left and non-CR on right for each cytokine).

Table E1 shows data summarizing the responses from subjects treated with the 1×10⁹ and 1.5×10⁹ cell doses. Subjects were allowed multiple dosing cycles to deepen responses, and subjects exhibiting a CR were allowed to receive an additional dosing cycle as consolidation cycle. Subjects that exhibited an initial clinical benefit and subsequent disease progression were allowed to receive retreatment.

TABLE E1
Clinical Responses in NHL Subjects

| | 1 × 10⁹ cells × 3 doses | | 1.5 × 10⁹ cells × 3 doses | |
|---|---|---|---|---|
| | ORR (CR, PR) | CR | ORR (CR, PR) | CR |
| ALL NHL | 5/6 (83%) | 4/6 (67%) | 3/4 (75%) | 3/4 (75%) |
| LBCL# | 1/2 | 1/2 | 1/2 | 1/2 |
| MCL | 1/1 | 1/1 | — | — |
| FL | 2/2 | 1/2* | 2/2 | 2/2 |
| MZL | 1/1 | 1/1 | — | — |

#LBCL includes DLBCL and FL3b
*PR deepened to CR after initial response determination
ALL, acute lymphoblastic leukemia; CR, complete response; DLBCL, diffuse large B-cell lymphoma; FL, follicular lymphoma; FL3b, follicular lymphoma grade 3b; LBCL, large B-cell lymphoma; MCL, mantle cell lymphoma; MZL, marginal zone lymphoma; NHL, non-Hodgkin lymphoma; ORR, overall response rate; PR, partial response.

The overall response rate (ORR) was 8/10 (80%) for the 1×10⁹ and 1.5×10⁹ cell dose levels, with 7 of those subjects achieving a CR. 4 of the 7 subjects achieved a CR after a single dosing cycle, and 3 subjects with initial PR deepened to CR after additional cycles (including one subject with FL treated with the 1×10⁹ cell dose level). 6 subjects received a consolidation cycle after achieving CR. The median interval between treatment cycles was 8 days, with lymphodepletion provided at the beginning of each 28-day cycle. 40% of eligible patients received the CD19 CAR-NK cells in the outpatient setting after the first cycle.

Follow-up analyses revealed multiple patients with response durability beyond 6 months, and one subject with follicular lymphoma (FL) treated with the 1×10⁹ dose had a PR that subsequently deepened to a CR. Peak concentration of cells trended higher in patients achieving a CR. Retreatment was planned for 2 subjects exhibiting an initial clinical response and subsequent disease progression. Nearly every subject who received CD19 CAR-NK cells exhibited a reduction in tumor size. A summary of clinical responses over time is shown by the swimmer plot in FIG. 4D.

In summary, the data are consistent with a finding that successive rounds of dosing cycles with CD19 CAR-NK cells are feasible and effective to achieve clinical responses.

### Example 2—Second Dosing Regimen for NK Cell Immunotherapy

As discussed in more detail herein, certain cancer types express selected markers in an elevated manner. In several embodiments, cytotoxic receptor constructs are generated according to sequences disclosed herein in order to specifically target a given cancer. For example, many cancers express elevated levels of CD19, including Non-Hodgkin's lymphoma (including various subtypes of NHL). Thus, as discussed in detail above, in several embodiments, CD19-targeting CAR constructs are provided. In several embodiments, the polynucleotides encoding those constructs are engineered to bi-cistronically express mblL15 (e.g., SEQ ID NO: 1 or SEQ ID NO: 53). A dosing regimen will be tested to evaluate the efficacy of cells expressing such constructs. In several embodiments, the dosing regimen will employ NK cells (from healthy donors) engineered to express the CD19-CAR. In several embodiments, the engineered NK cells will express the cytotoxic receptor encoded by SEQ ID NO: 3 (including degeneracies or codon-optimized versions of SEQ ID NO: 3). In several embodiments, the engineered NK cells will express the cytotoxic receptor comprising the amino acid sequence of SEQ ID NO: 43 (e.g., SEQ ID NO: 4) (and, optionally mblL15 comprising the amino acid sequence of SEQ ID NO: 44 (e.g., SEQ ID NO: 40)).

The dosing regimen was designed to evaluate engineered NK cells administered three times in a 28-day dosing cycle to treat Non-Hodgkin's lymphoma (NHL), including LBCL (e.g., aggressive LBCL). Subjects with LBCL include those that are CD19 CAR T cell therapy naive and experienced. The dosing cycle was preceded by a conditioning phase during which a subject underwent lymphodepletion (using cyclophosphamide (500 mg/m²) and fludarabine (30 mg/m²) at day-5, day-4, and day-3). At day 0, each subject received the first dose of 1.5×10⁹ CD19 CAR-NK cells. Dose 2 was administered at day 7 and dose 3 was administered at day 14 (both dose 2 and 3 are 1.5×10⁹ CD19 CAR-NK cells). At approximately day 28, outcome measures were assessed.

Primary endpoints included: (1) incidence, nature, and severity of treatment related adverse events will be evaluated, with an adverse event defined as any unfavorable and unintended sign including clinically significant abnormal laboratory findings, symptom or disease, measured 30 days after last dose of the NK cells; and (2) proportion of subjects experiencing dose-limiting toxicities (DLTs) of the NK cells, with DLTs defined as adverse events attributable to treatment that occur during Cycle 1 and meet protocol specified criteria, measured 28 days from first dose of NK cells.

Secondary outcome measures included evaluation of: (1) pharmacokinetic parameters in the context of the immune system, including but not limited to maximum concentration (Cₘₐₓ), time to reach maximum concentration (Tₘₐₓ), area under the concentration-time curve (AUC), half-life (t_{1/2}), and duration of persistence of the CD19 CAR-NK cells in the peripheral blood and other target tissues such as bone marrow; (2) humoral and cellular immunogenicity against the CD19 CAR-NK cells; (3) changes in serum cytokine levels such as interferon-gamma (IFN-γ) and other host responses to CD19 CAR-NK cells in peripheral; (4) best overall response rates in dose finding and safety lead-in cohorts; and/or (5) other antitumor measurements, which may include duration of response (DOR), time-to-first response, time-to-best response, bridge-to-transplant rate, event-free survival (EFS), progression free survival (PFS), and overall survival (OS) using standard disease specific response assessment criteria.

It is believed that the administration of three doses of engineered NK cells expressing a CD19-targeting CAR and also expressing mblL15 will be favorably tolerated and show limited adverse events. It is also believed that the administration of three doses of engineered NK cells expressing a CD19-targeting CAR and also expressing mblL15 will result in limited DLTs. It is believed that the increased concentration of cyclophosphamide will enhance the anti-cancer effects of the engineered NK cells. It is believed that the NK cells will show an extended half-life as well as enhanced duration of persistence. It is believed that the NK cells will induce limited host immune response and a clinically meaningful objective response rate (e.g.,) reductions in tumor burden.

### Example 3—Basis for Combination Dosing Regimens

Combination therapies using therapeutic agents having non-mutually exclusive (or mutually exclusive) mechanisms of action may allow for synergistic activities and enhanced cancer immunotherapies. As discussed in more detail herein, certain cancer types express selected markers in an elevated manner. In several embodiments, cytotoxic receptor constructs are generated according to sequences disclosed herein in order to specifically target a given cancer. For example, many cancers express elevated levels of CD19, including Non-Hodgkin's lymphoma (including various subtypes of NHL). In several embodiments, anti-CD20 antibodies (or biosimilars) are utilized in conjunction with engineered NK cells as provided for herein to enhance the anti-tumor effect. In several embodiments, the anti-CD20 antibody is rituximab, obinutuzumab, or combinations thereof. Anti-CD20 antibodies (e.g., monoclonal antibodies like rituximab or obinutuzumab) can function to impart cytotoxic effects in various ways. For example, direct binding of CD20 monoclonal antibodies to a tumor cell may initiate crosslinking of multiple CD20 molecules, resulting in cell-death via induction of non-classical apoptosis. Likewise, binding of the antibodies may result in activation of the complement system, resulting in complement-dependent cytotoxicity. Additionally, immune effector cells (e.g., NK cells) may recognize opsonized tumor cells by FcγRs expressed on the immune effector cells, thereby initiating antibody dependent cell-mediated cytotoxicity (ADCC). Also antibody-initiated complement activation may lead to deposition of complement cleavage fragments, which may enhance tumor killing through complement receptors, known as complement-enhanced ADCC. Additionally, FcγR may serve as a crosslinking platform to enhance antigen signaling in the tumor cells.

To investigate the possible enhanced effectiveness of a CD19-CD20 targeting combination therapy, the effectiveness of various compositions disclosed herein was tested on cell lines derived from B cell malignancies expressing CD19 and CD20. These included, as non-limiting examples, Raji lymphoblast-like cells (Burkitt lymphoma), DOHH-2 (follicular centroblastic/centrocytic lymphoma), and EHEB (B-CLL chronic lymphocytic leukemia). Cytotoxicity and ADCC were measured in 4-hour and extended assays when CD19 CAR NK cells were cultured with tumor cells in the presence or absence of anti-CD20 monoclonal antibodies (non-limiting examples of which are rituximab and obinutuzumab.

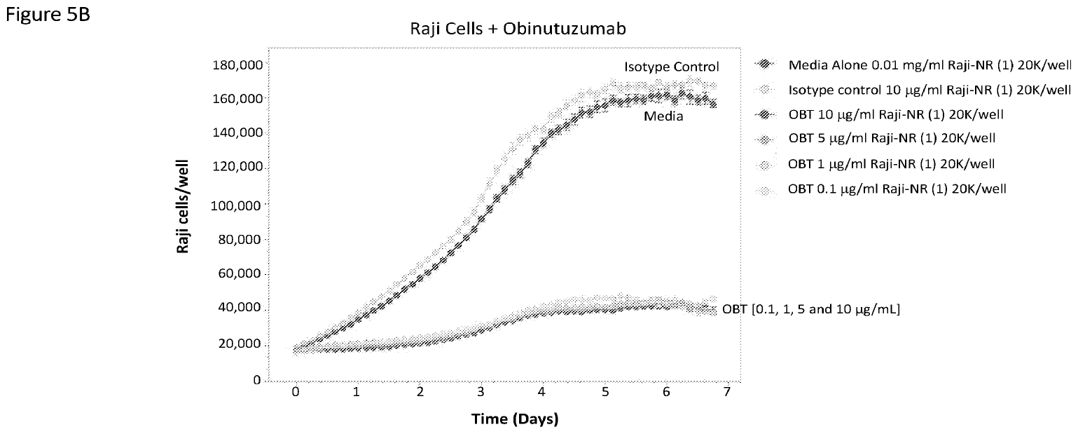

FIG. 5A shows a plot of cytotoxicity of rituximab at various concentrations against Raji tumor cells in vitro. Raji cells were cultured with rituximab at the indicated concertation over 7 days in culture. FIG. 5B shows analogous data for obinutuzumab. These data show that both of these anti-CD20 antibodies (despite the Type I mechanism of action of rituximab and the Type II mechanism of action of obinutuzumab) are active against Raji tumor cells (as a non-limiting example of a B-cell tumor). Both antibodies exhibited control of tumor growth over the assay, with only the lowest concentration (0.1 μg/mL of rituximab allowing a modest increase in tumor cell number.

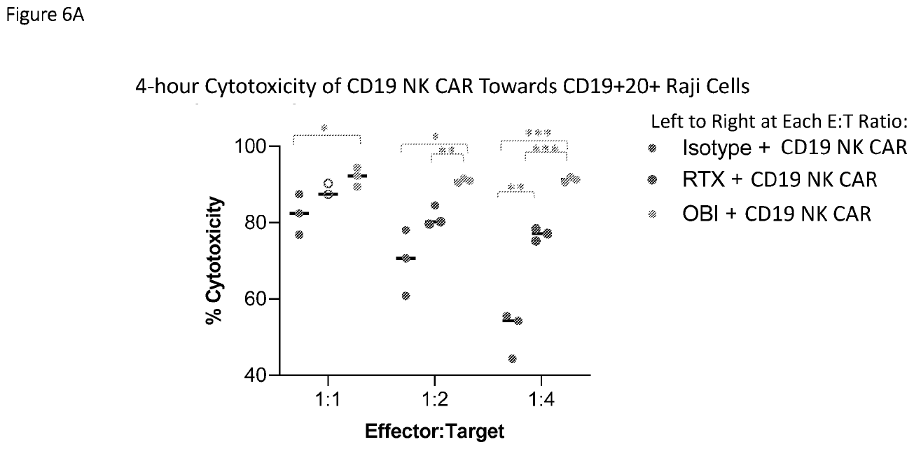

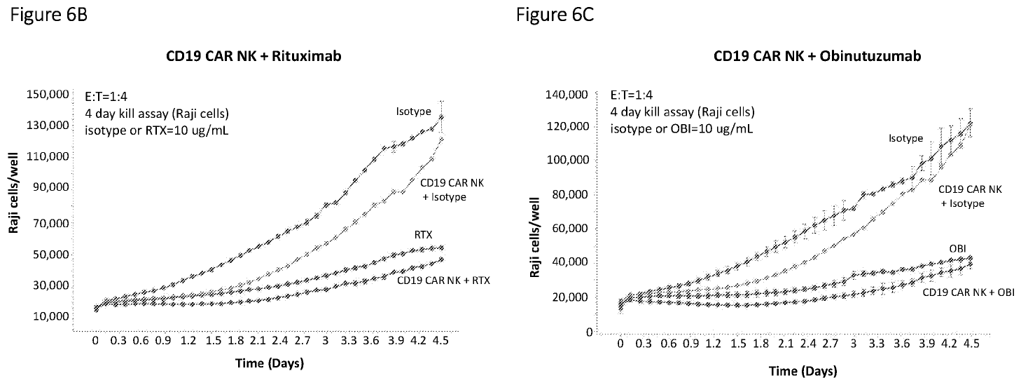

To investigate the possible enhancements (e.g., synergy between the agents) using a combination therapy a co-culture was performed in which Raji cells were co-cultured with an anti-CD20 antibody as well as NK cells engineered to express a CD19-targeting CAR, as provided for herein, for a duration of four hours. FIG. 6A shows summary data of the resultant cytotoxicity, with Effector:Target (E:T) ratios of 1:1 (left) 1:2 (center) or 1:4 (right). In each dot plot, the left-most group is CD19 CAR NK cells and an antibody isotype control, the middle is CD19 CAR NK cells with rituximab (1 μg/ml), and the right is CD19 CAR NK cells with obinutuzumab (1 μg/ml). As shown in FIG. 6A, combination therapy with CD19 CAR NK cells and either of the two non-limiting examples of CD20 antibodies appears to increase the cytotoxic effects of CD19 NK CAR against CD19+/CD20+ target cells, such as the non-limiting example Raji cells used here. As the E:T ratio decreased, the contribution of the antiCD20 antibodies became more discernable. While the isotype control group at 1:4 E:T was still able to kill-50% of the target cells, the addition of rituximab increased the cytotoxicity to nearly the same level as that seen in the 1:2 E:T group (˜80%). Addition of obinutuzumab surprisingly resulted in even more increased cytotoxicity of CD19 NK CAR (˜90%; about the same as the 1:1 E:T isotype control. N=3, Representative Donor is shown, E:T=1:4. *p<0.05, ** p<0.01, *** p<0.001, unpaired t-test). These data show that a combination approach of targeting CD19 and CD20 result in unexpected enhancements in cytotoxicity.

Building on this initial data, longer duration experiments were performed. FIG. 6B shows the cytotoxicity profile of rituximab (at 10 μg/mL) in combination with CD19 CAR NK cells in a 4 day kill assay using Raji cells at a 1:4 E:T ratio. As expected, the isotype antibody control and CD19 CAR NK cells (because of the low E:T ratio) allowed Raji cell expansion. Rituximab alone seemed to initially prevent Raji cell expansion, with modest increases over the course of the assay. The combination of CD19 CAR NKs and rituximab exhibited the most robust control of tumor growth. Similar results were seen using obinutuzumab and CD19 CAR NKs (see FIG. 6C).

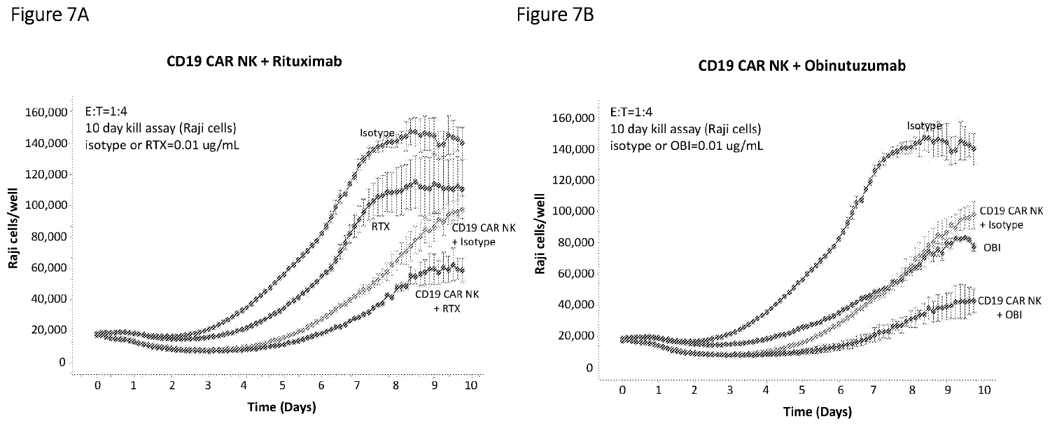

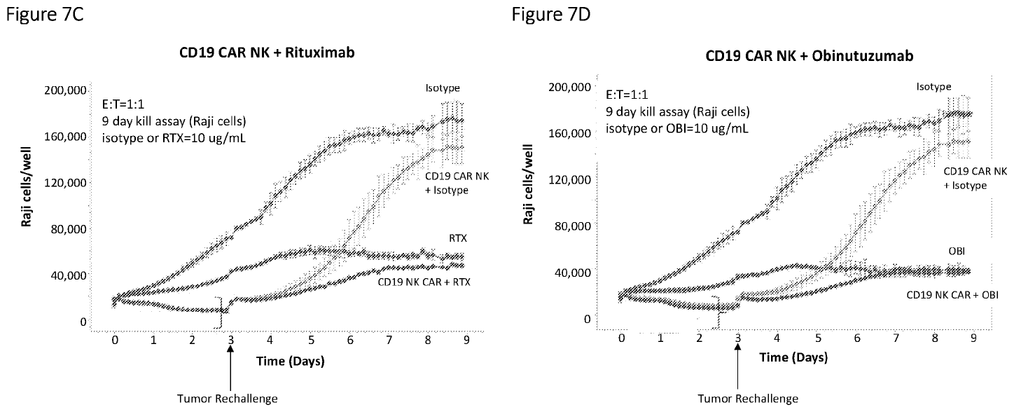

Interestingly, a further enhancement of the combination was observed when a lower concentration of either rituximab or obinutuzumab was used in combination with CD19 CAR NK cells with a 10-day co-culturing assay. These data are shown in FIGS. 7A and 7B, respectively. The antibodies were used at 0.01 μg/mL in these assays, again with a 1:4 E:T ratio. In contrast to the data of FIGS. 6B-6C, where the combination of the antibodies and NK cells yielded similar control of tumor growth as compared to antibodies alone, these data show a marked separation between those groups. The combination of the respective anti-CD20 antibodies and CD19 CAR NK cells markedly improved tumor control, evidencing potential synergy between the two agents, made particularly surprising in view of a reduction in the concentration of the antibody.

Further experiments were done to assess the efficacy of combination therapies when CD19 CAR NK cells were rechallenged with additional Raji cells. At Day 3 after inception of co-culture at a 1:1 E:T ratio, 10×10³ additional Raji cells were added to the CD19 CAR NK cells and cultured for an additional 6 days (total of 9 days) with either an isotype control antibody, rituximab, or obinutuzumab (each at 10 μg/ml). As shown in FIG. 7C, both rituximab alone and rituximab in combination with CD19 CAR NK cells exhibited effective control of tumor cell expansion, with the combination therapy providing modestly greater control than the CD20 antibody alone. Likewise, in FIG. 7D, both obinutuzumab and obinutuzumab in combination with CD19 CAR NK cells exhibited effective control of tumor cell expansion.

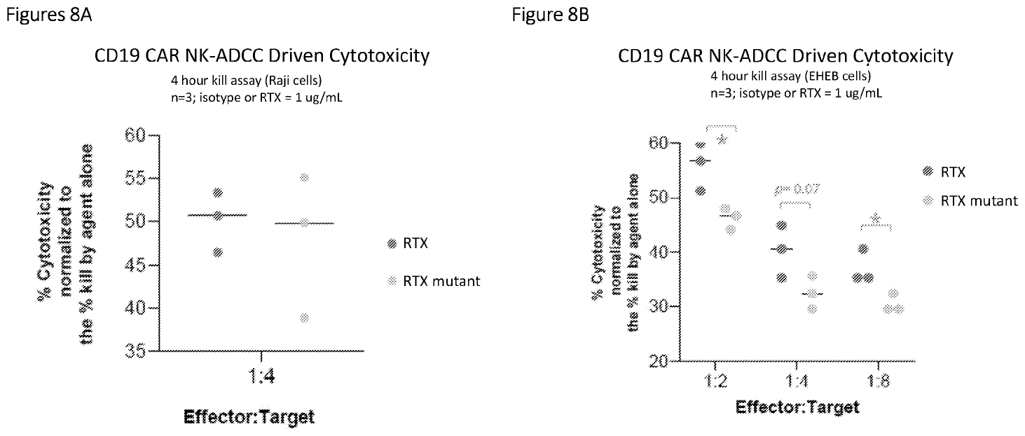

In additional initial experiments into the potential mechanisms of action, assays were performed using a normal rituximab as well as a mutant rituximab. The mutant antibody has, within the constant region of the human IgG1, asparagine residues that comprise putative glycosylation sites are substituted by glutamine residues. This results in a non-glycosylated antibody which exhibits compromised ADCC function. FIG. 8A shows the normalized cytotoxic effect of CD19 CAR NKs when used in combination with normal rituximab (left) or mutant rituximab (right). This assay used Raji cells at 1:4 E:T ratio and a rituximab concentration of 1 μg/mL. As can be seen, the cytotoxicity of the CD19 CAR NK cells is unchanged, indicating that, at least for this cell type, the NK cells kill independently of ADCC as a mechanism of action. In contrast, FIG. 8B shows corresponding data (although at various E:T ratios) using an EHEB tumor cell line. Some tumor cells exhibit resistance to certain therapies based on their mechanism of action, for example by resistance to complement-dependent cytotoxicity. This means that knowledge of which mechanisms are at play with a given therapy could be helpful in terms of performing an in vitro screen prior to administering a therapy. FIG. 8B shows data related ADCC disruption and cytotoxicity against EHEB cells. As shown, at each E:T ratio, the use of the mutant form of rituximab significantly decreases the degree of cytotoxicity exhibited by the CD19 CAR NK cells. These data suggest that screens for various tumor susceptibility prior to inception of a combination therapy could be useful to predict the success of the therapy.

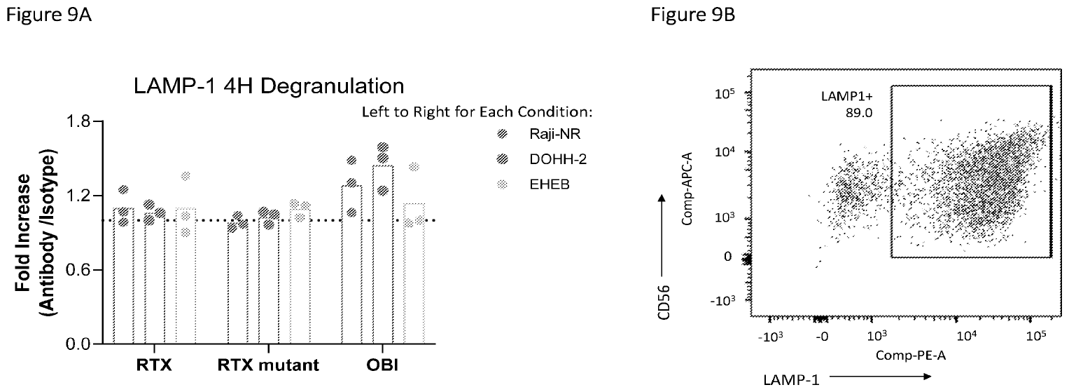

Further investigation was performed with respect to the mechanism of action(s) at play in combination therapies. CD19 CAR NK cells were co-cultured at a 1:1 ratio of target cells (either Raji (lymphoblast-like), DOHH-2 (follicular lymphoma) or EHEB (B-Lymphoblastoid) cells) in the presence of 1 μg/mL of anti-CD20 antibodies (rituximab, mutant rituximab as in FIG. 8, or obinutuzumab) and stained for LAMP-1 after 4 hours. LAMP-1 (also known as CD107a) is a marker of NK cell degranulation. Increases in LAMP-1 expression cells in the antibody groups was normalized to the isotype-antibody group. FIG. 9A shows LAMP-1 expression from 3 donors after co-culture with one of the various tumor cell types. These data show that obinutuzumab appears to enhance NK cell degranulation, regardless of the tumor type. A combination of phorbol myristate acetate (PMA) and ionomycin was used to activate NK cells (in the absence of tumor cells). NK cell LAMP-1 expression was measured. As shown in FIG. 9B, the stimulation of NK cells with this combination of agents induced a substantial increase in LAMP-1 expression, with nearly 90% of the NK cells expressing elevated LAMP-1 levels. Like PMA/ionomycin, tumor cells induce NK cell activation and degranulation, which appears to be enhanced by the addition of the non-limiting example anti-CD20 antibody obinutuzumab, supporting the principle of combination therapy and enhanced cytotoxicity, as provided for herein.

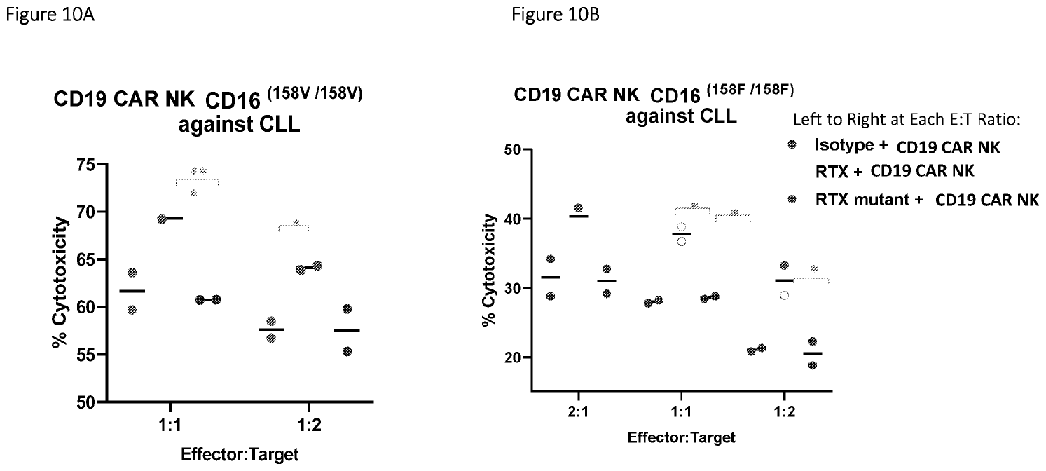

It is known that the non-limiting examples of CD20-targeted antibodies used in these experiments can mediate antibody-dependent cellular cytotoxicity (ADCC), which is a key effector mechanism of NK cells and is facilitated by the Fc receptor, CD16a. A common polymorphism is found in CD16 that affects affinity for Fc and can influence ADCC responses. Specifically, the polymorphism at position 158 of phenylalanine (158F) or valine (158V) results in CD16 with decreased or increased affinity for Fc, respectively. To determine the potential impact of the CD16 polymorphism on combination therapy, leukemic cells from 2 CLL subjects were incubated with CD19 CAR NK cells from donors expressing the 158V higher affinity CD16 variant (FIG. 10A) or the 158F lower affinity CD16 variant (FIG. 10B) in the presence of 1 μg/mL of anti-CD20 antibodies (rituximab or mutant rituximab as in FIG. 8) or corresponding irrelevant isotype control. Co-cultures were for 4 hours at the indicated E:T ratios. Data shown are an average of % cytotoxicity from the two CLL subjects. Data is presented as the normalized frequency of target cells remaining, where target cells+ isotype control without effector cells is =100%. *p<0.05, ***p<0.001, unpaired t-test. As can be seen from FIGS. 10A and 10B, the addition of rituximab to CD19 CAR NK cells enhanced the cytotoxic activity of the CD19 CAR NK cells as compared to control or when a mutant rituximab is used. This increase occurs irrespective of whether the CD16 variant is the high or low affinity variant. While the high affinity CD16 variant NK cells exhibited greater overall cytotoxicity, even the low affinity variant NK cells exhibited significant increases in combination with rituximab over isotype control at 1:1 and 1:2 E:T ratios. Together these data support the use of anti-CD20 antibodies in combination with CD19 CAR NK cells in order to provide enhanced therapies for treating B cell malignancies.

### Example 4—Combination Dosing Regimen for NK Cell Immunotherapy

As discussed in more detail herein, certain cancer types express selected markers in an elevated manner. In several embodiments, cytotoxic receptor constructs are generated according to sequences disclosed herein in order to specifically target a given cancer. For example, many cancers express elevated levels of CD19, including Non-Hodgkin's lymphoma (including various subtypes of NHL). Thus, as discussed in detail above, in several embodiments, CD19-targeting CAR constructs are provided. In several embodiments, the polynucleotides encoding those constructs are engineered to bi-cistronically express mblL15 (e.g., SEQ ID NO: 1 or SEQ ID NO: 53). A dosing regimen will be tested to evaluate the efficacy of cells expressing such constructs. In several embodiments, the dosing regimen will employ NK cells engineered to express the CD19-CAR. While this study will employ haplomatched NK cells, as discussed herein, in several embodiments, the NK cells will be off the shelf allogeneic engineered NK cells (derived from an unrelated donor) and may optionally be compared against matched doses of haplo-matched related donor-derived engineered NK cells. In several embodiments, the engineered NK cells will express the cytotoxic receptor encoded by SEQ ID NO: 3 (including degeneracies or codon-optimized versions of SEQ ID NO: 3). In several embodiments, the engineered NK cells will express the cytotoxic receptor comprising the amino acid sequence of SEQ ID NO: 43 (e.g., SEQ ID NO: 4) (and, optionally mblL15 comprising the amino acid sequence of SEQ ID NO: 44 (e.g., SEQ ID NO: 40). In addition, tumor cells may express elevated levels of another marker (e.g., not CD19, which is targeted by the CAR mentioned above) that can be targeted by another agent. For example, several tumor types the show increased CD19 also exhibit elevated levels of CD20. Thus, in some embodiments, a CD19-targeting CAR NK cell population is administered in combination with a CD20-targeting agent, which could be an antibody (such as a monoclonal antibody like rituximab or Obinutuzumab) or a CD20-targeting CAR NK (and/or T) cell.

The dosing regimen will be designed to evaluate engineered NK cells that will be administered three times in a 28-day dosing cycle to treat Non-Hodgkin's lymphoma, including LBCL (e.g., aggressive LBCL). The dosing regimen will be optionally administered on an outpatient basis. Subjects include those that are CD19 CAR T cell therapy naive and experienced. The dosing cycle will be preceded by a conditioning phase during which a subject will undergo lymphodepletion (using cyclophosphamide (500 mg/m²) and fludarabine (30 mg/m²) at day-5, day-4, and day-3). Three days prior to the first administration of CD19-targeting CAR NK cells (day-3), subjects will receive a single dose of an anti-CD20 monoclonal antibody, e.g. rituximab at a dose of 375 mg/m²). At day 0, each subject will receive the first dose of 1.5×10⁹ CD19 CAR-NK cells. Dose 2 will be administered at day 7 and dose 3 will be administered at day 14 (both dose 2 and 3 are 1.5×10⁹ CD19 CAR-NK cells). At approximately day 28, outcome measures will be assessed.

Primary Endpoints will include: (1) incidence, nature, and severity of treatment related adverse events will be evaluated. An adverse event is any unfavorable and unintended sign including clinically significant abnormal laboratory findings, symptom or disease. This was measured 30 days after last dose of the NK cells and (2) proportion of subjects experiencing dose-limiting toxicities (DLTs) of the NK cells, with DLTs defined as adverse events attributable to treatment that occur during Cycle 1 and meet protocol specified criteria. This will be measured 28 days from first dose of NK cells.

Secondary outcome measures will include evaluation of: (1) pharmacokinetic parameters in the context of the immune system, including but not limited to maximum concentration (Cₘₐₓ), time to reach maximum concentration (Tₘₐₓ), area under the concentration-time curve (AUC), half-life (t_{1/2}), and duration of persistence of the CD19 CAR-NK cells in the peripheral blood and other target tissues such as bone marrow; (2) humoral and cellular immunogenicity against the CD19 CAR-NK cells; (3) changes in serum cytokine levels such as interferon-gamma (IFN-γ) and other host responses to CD19 CAR-NK cells in peripheral; (4) best overall response rates in dose finding and safety lead-in cohorts; and/or (5) other antitumor measurements, which may include duration of response (DOR), time-to-first response, time-to-best response, bridge-to-transplant rate, event-free survival (EFS), progression free survival (PFS), and overall survival (OS) using standard disease specific response assessment criteria.

It is believed that the administration of three doses of engineered NK cells expressing a CD19-targeting CAR and also expressing mblL15 will be favorably tolerated and show limited adverse events. It is also believed that the administration of three doses of engineered NK cells expressing a CD19-targeting CAR and also expressing mblL15 will result in limited DLTs. It is believed that the increased concentration of cyclophosphamide will enhance the anti-cancer effects of the engineered NK cells. It is believed that the increased concentration of cyclophosphamide will enhance the anti-cancer effects of the engineered NK cells. It is also believed that the use of the anti-CD20 antibody will enhance the overall therapeutic outcome based on different, but synergistic, mechanism of action as compared to the CAR NK cells. It is believed that the NK cells will show an extended half-life as well as enhanced duration of persistence. It is believed that the NK cells will induce limited host immune response and a clinically meaningful objective response rate (e.g.,) reductions in tumor burden.

This is a prophetic example.

### Example 5—Treatment of CD58 Knockout Cells with CD19 CAR NK Cells

The potency of natural killer (NK) cells and T cells expressing non-limiting embodiments of a CD19-directed CAR was assessed in vivo and in vitro against target tumor cells knocked out for CD19, CD58, or CD19 and CD58.

Natural killer (NK) cells were isolated via immunoaffinity from peripheral blood mononuclear cells (PBMCs) from healthy human donors. The isolated NK cells were transduced with a viral vector encoding a CD19-directed CAR and a membrane-bound interleukin-15 (mblL15; e.g., SEQ ID NO:44), separated by a T2A (e.g., SEQ ID NO:36). The CD19-directed CAR contained an anti-CD19 scFv (e.g., SEQ ID NO:6), a CD8 alpha hinge (e.g., SEQ ID NO:28) and transmembrane region (e.g., SEQ ID NO:30), and an intracellular signaling domain containing an OX40 co-stimulatory region (e.g., SEQ ID NO:32) and CD3zeta (e.g., SEQ ID NO:34). Separately, T cells were isolated via immunoaffinity from PBMCs from healthy human donors and subsequently transduced with a viral vector encoding a CD19-directed CAR. The CD19-directed CAR contained the FMC63 anti-CD19 scFv, a CD8 alpha transmembrane domain, and an intracellular signaling domain containing a 4-1BB co-stimulatory region and CD3zeta.

Nalm6/Luc2.eGFP target tumor cells were stably knocked out for CD19 (CD19KO), CD58 (CD58KO), or CD19 and CD58 (CD19KOCD58KO) via genetic editing with Cas9 and commercially available CD19- and CD58-targeting gRNA sequences. Unedited Nalm6/Luc2.eGFP cells expressing CD19 and CD58 (WT) served as controls.

### A. Characterization of CD19 and CD58 Knockout Tumor Cells

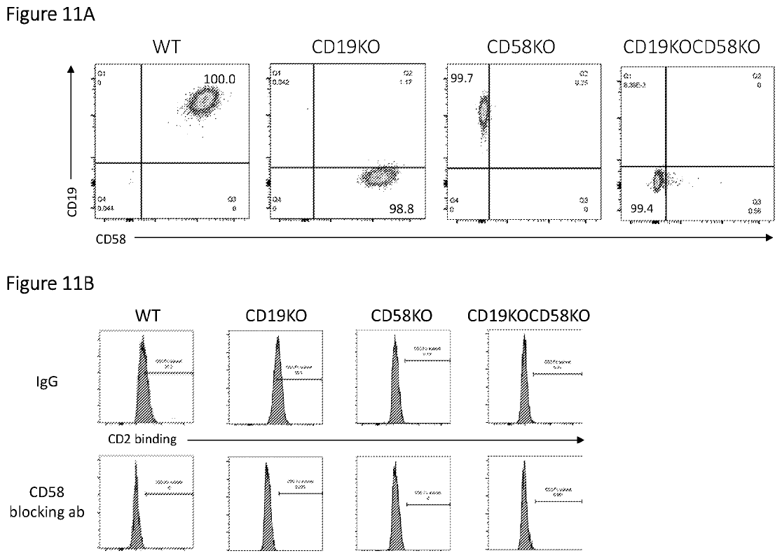

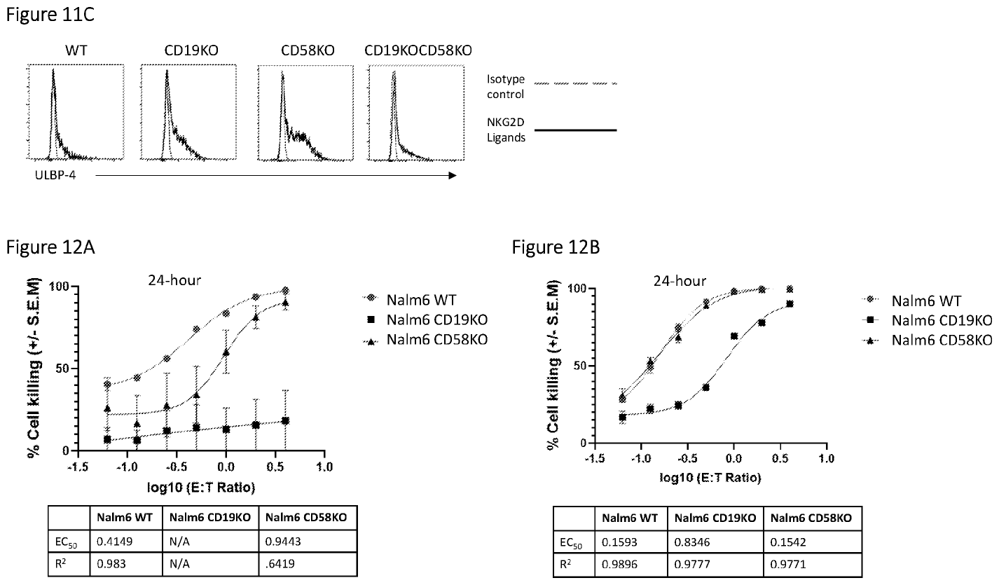

Flow cytometric analysis revealed cell surface expression of CD19 and CD58 to be absent in cells stably knocked out for CD19 and CD58, respectively, including after prolonged culture of the cells (representative images shown in FIG. 11A). Binding of CD2 was assessed in CD19KO, CD58KO, and CD19/CD58 KO cells by flow cytometry. Briefly, target cells were first incubated with IgG (to allow for CD2-CD58 binding) or a CD58 blocking antibody (to block CD2-CD58 binding), then subsequently incubated with a biotin-labeled CD2-Fc fusion protein. CD2 binding was detected by staining with a PE anti-biotin antibody. CD2 binding was observed to be absent in CD58KO and CD19KOCD58KO cells, as well as in CD19KO and WT cells incubated in the presence of the CD58 blocking antibody (representative images shown in FIG. 11B).

Cell surface expression of NKG2D ligands was assessed by flow cytometry in target cells. Staining with anti-MICA, MICB, ULBP-1, ULBP-3, and ULBP-2/5/6 antibodies revealed expression of these ligands to be comparable among WT, CD19KO, CD58KO, and CD19KOCD58KO cells (data not shown). Expression of ULBP-4 was observed to be increased in CD19KO and CD58KO cells, as compared to CD19KOCD58KO and WT cells (representative images shown in FIG. 11C).

Karyotyping and cell identification assays of Nalm6/Luc2.eGFP WT, CD19KO, CD58KO, and CD19KOCD58KO cells showed that the WT cells were genetically similar to the CD19KO, CD58KO, and CD1 9KOCD58KO cells

Together, these data indicate that CD19 and CD58, separately or in combination, can be stably knocked out in target tumor cells without significant effects on aspects of gross phenotype and genotype.

B. In Vitro Cytotoxicity of CD19 CAR NK and CD19 CAR T Cells against CD19 and CD58 Knockout Tumor Cells

CD19 CAR-expressing NK and T cell populations incubated with target tumor cells at various effector:target (E:T) ratios, and cytotoxicity against the target cells was evaluated by BrightGlo® and Incucyte® assays at various timepoints.

Knockout of CD19 effectively abolished the cytotoxicity of CD19 CAR T cells against target tumor cells in 24- and 96-hour BrightGlo® assays (FIG. 12A; 96-hour time point not shown). CD58 knockout was also observed to significantly decrease the cytotoxicity of CD19 CAR T cells at the 24- and 96-hour time points across almost all E:T ratios tested (FIG. 12A; 96-hour time point not shown). These data are consistent with findings by others that in vitro and in vivo CD19 CAR T cell potency is compromised by CD58 loss, and CD58 loss or mutation is associated with worse outcomes in large B-cell lymphoma (LBCL) patients (Majzner et al., Blood (2020) 136(Suppl. 1):53-54).

By contrast, while CD19 knockout was observed to significantly decrease the cytotoxicity of CD19 CAR NK cells against target tumor cells in a 24-hour BrightGlo® assay, CD58KO was not observed to affect the cytotoxicity of the CD19 CAR NK cells (FIG. 12B).

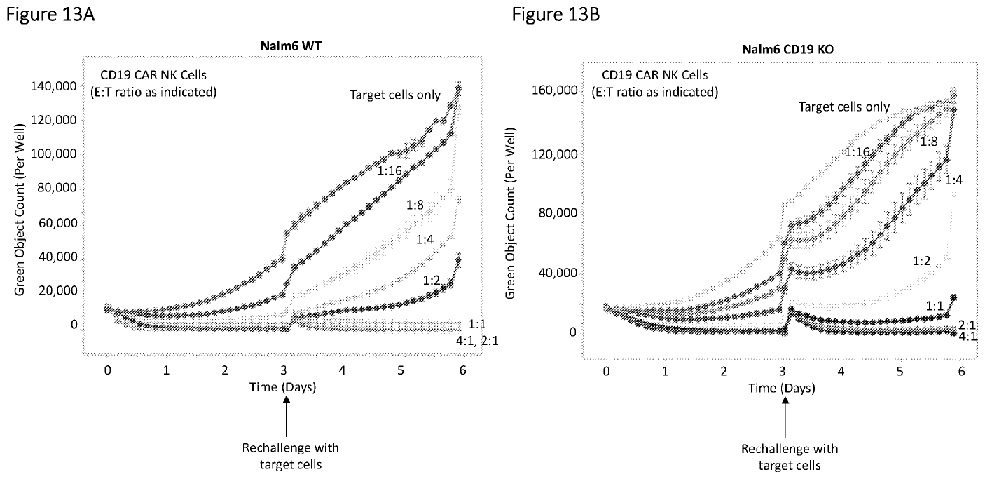

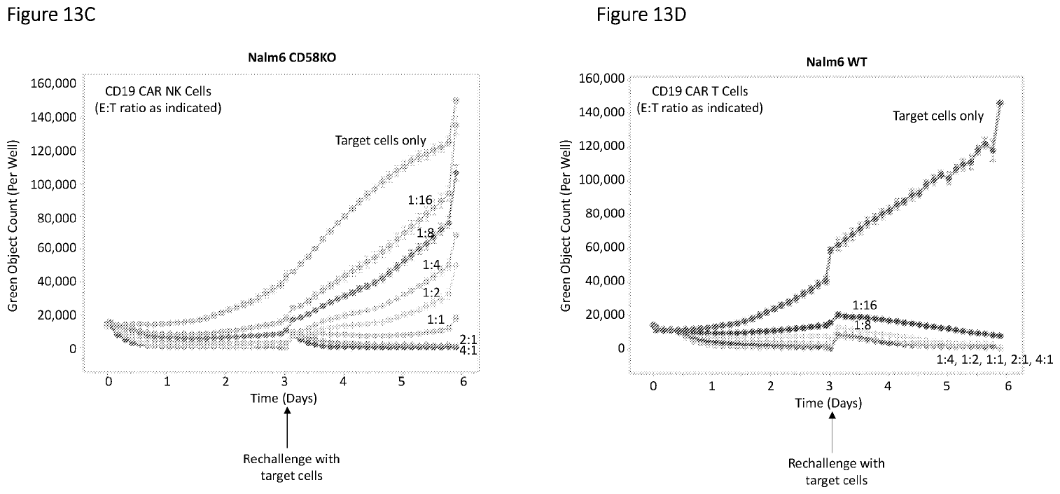

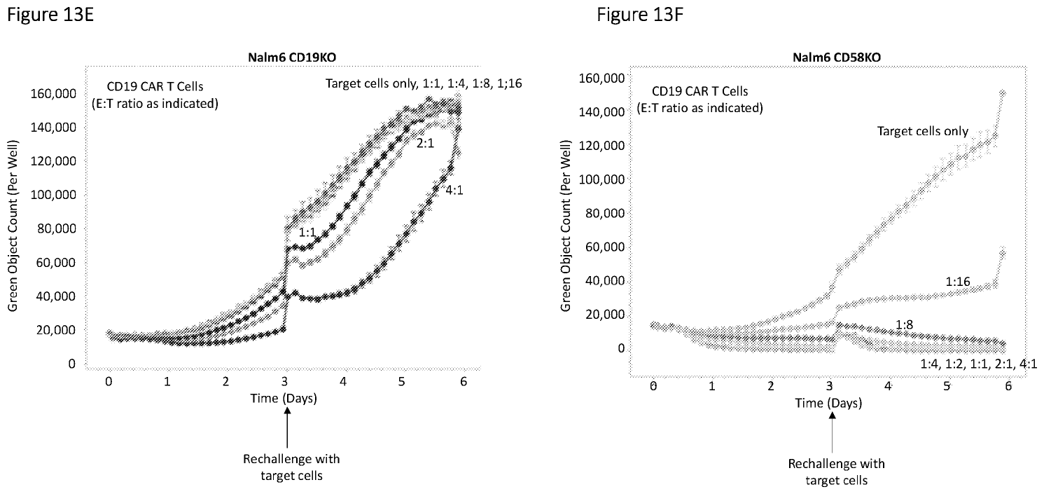

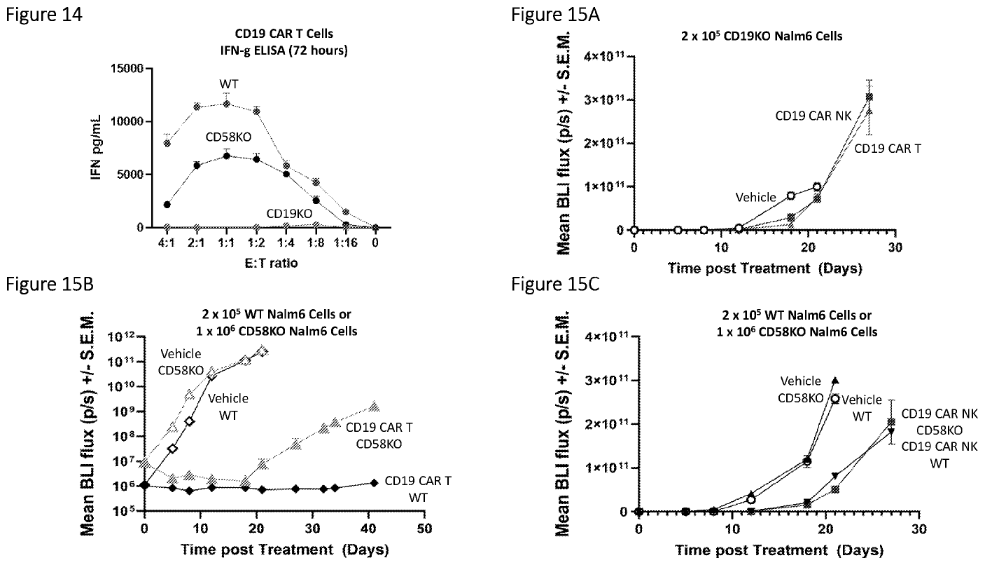

Incucyte® assays were performed to monitor target cell proliferation over time by co-culturing 20,000 target cells with CD19 CAR T cells or CD19 CAR NK cells for 72 hours at various E:T ratios. After 72 hours, an additional 10,000 target cells were added to co-culture to rechallenge the CD19 CAR T cells or CD19 CAR NK cells. Target cell proliferation was monitored for an additional 72 hours after rechallenge. As in the BrightGlo® assays, CD19 CAR T cells demonstrated substantial decreases in potency against CD19KO target cells, as well as decreased potency against CD58KO target cells at higher E:T ratios (e.g., 1:16), whereas the potency of CD19 CAR NK cells was only observed to be decreased against CD19KO cells (FIGS. 13A-F). Additional analyses confirmed that the sensitivity of target cells to CD19 CAR NK cells was not significantly affected by CD58 knockout at the time of rechallenge or 72 hours after rechallenge, whereas the sensitivity of target cells to CD19 CAR T cells was significantly decreased by CD58 knockout at both time points (data not shown). Supernatants from the co-culture of CD19 CAR T cells and target cells were taken at the time of rechallenge and T cell production of interferon-gamma was assessed by ELISA. Across various E:T ratios, the CD19 CAR T cells produced significantly less interferon-gamma in the presence of CD58KO target cells, and a negligible amount of interferon-gamma in the presence of CD19 KO cells (FIG. 14).

C. In Vivo Cytotoxicity of CD19 CAR NK and CD19 CAR T Cells against CD19 and CD58 Knockout Tumor Cells

Efficacy of the CD19 CAR-expressing NK and T cell populations described in this Example was measured against a murine Nalm6 tumor model. On Day-1, NOD scid gamma mice (NSG) mice were injected with 2×10⁵ WT or CD19KO luciferase-labeled Nalm6 cells or 1×10⁶ CD58KO luciferase-labeled Nalm6 cells. Mice were subsequently injected with a single dose of 1×10⁹ CD19 CAR NK cells or 1.2×10⁶ CD19 CAR T cells (or vehicle, as a control) on Day 0. Tumor volume was monitored for 28 days by bioluminescence imaging (BLI).

Substantial CD19KO tumor cell growth was observed in mice treated with CD19 CAR NK or CD19 CAR T cells (FIG. 15A). Whereas CD19 CAR T cells exhibited decreased tumor control against CD58KO tumor cells compared to WT tumor cells (FIG. 15B), tumor control by CD19 CAR NK cells was not decreased against CD58KO tumor cells (FIG. 15C).

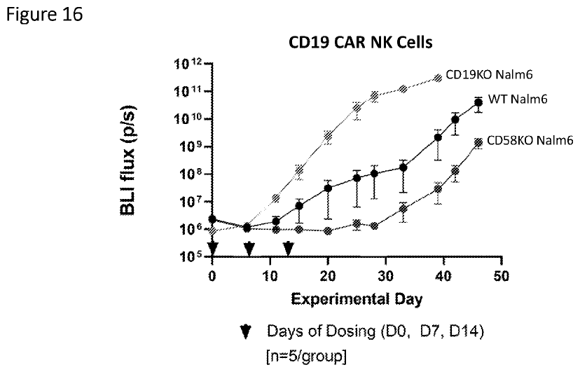

In a further in vivo experiment, NSG mice were injected with 2×10⁵ WT or CD19KO luciferase-labeled Nalm6 cells or 1×10⁶ CD58KO luciferase-labeled Nalm6 cells. Mice were subsequently injected with a single dose of 1×10⁹ CD19 CAR NK cells or vehicle on each of Days 0, 7, and 14. Tumor volume was monitored for 61 days by BLI. Consistent with other findings described herein, CD19 CAR NK cells were observed to have decreased tumor control against CD19KO tumor cells compared to WT tumor cells (FIG. 16). Notably, in this longer-term experiment, CD19 CAR NK cells were observed to have significantly greater tumor control against CD58KO tumor cells compared to WT tumor cells (FIG. 16).

Without wishing to be bound by theory, these data are consistent with a finding that the activation and antitumor activity of CD19 CAR NK cells is not compromised by CD58 loss or mutation in tumor cells.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 90%” includes “90%.” In some embodiments, at least 95% sequence identity or homology includes 96%, 97%, 98%, 99%, and 100% sequence identity or homology to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence. Any titles or subheadings used herein are for organization purposes and should not be used to limit the scope of embodiments disclosed herein.

Articles such as “a”, “an”, “the” and the like, may mean one or more than one unless indicated to the contrary or otherwise evident from the context. The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. Embodiments are provided in which exactly one member of the group is present, employed in, or otherwise relevant to a given product or process. Embodiments are provided in which more than one, or all of the group members are present, employed in, or otherwise relevant to a given product or process. Any one or more claims may be amended to explicitly exclude any embodiment, aspect, feature, element, or characteristic, or any combination thereof. Any one or more claims may be amended to exclude any agent, composition, amount, dose, administration route, cell type, target, cellular marker, antigen, targeting moiety, or combination thereof.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

Sequence

| SEQ ID | | |
|---|---|---|
| NO | Sequence | Description |
| 1 | atggccttaccagtgaccgccttgctcctgccgctggccttgctgctccacg | CD19 CAR (nt) |
| | ccgccaggccggatattcagatgacccagagcccgagcagcctgagcgcgag | |
| | cgtgggcgatcgcgtgaccattacctgccgcgcgagccaggatattagcaaa | |
| | tatctgaactggtatcagcagaaaccgggcggcaccgtgaaactgctgattt | |
| | atcataccagccgcctgcatagcggcgtgccgagccgctttagcggcagcgg | |
| | cagcggcaccgattttaccctgaccattagcagcctgcagccggaagatatt | |
| | gcgacctatttttgccagcagggcaacaccctgccgtatacctttggcggcg | |
| | gcaccaaactggaaattaccggtggcggtggctcgggcggtggtgggtcggg | |
| | tggcggcggatctcaggtgcagctgcaggaaagcggcccgggcctggtgaaa | |
| | ccgagccagaccctgagcctgacctgcaccgtgagcggcgtgagcctgccgg | |
| | attatggcgtgagctggattcgccagccgccgggcaaaggcctggaatggat | |
| | tggcgtgatttggggcagcgaaaccacctattataacagcgcgctgaaaagc | |
| | cgcctgaccattagcaaagataacagcaaaaaccaggtgagcctgaaactga | |
| | gcagcgtgaccgcggcggataccgcggtgtattattgcgcgaaacattatta | |
| | ttatggcggcagctatgcgatggattattggggccagggcaccagcgtgacc | |
| | gtgagcagcaccacgacgccagcgccgcgaccaccaacaccggcgcccacca | |
| | tcgcgtcgcagcccctgtccctgcgcccagaggcgtgccggccagcggcggg | |
| | gggcgcagtgcacacgagggggctggacttcgcctgtgatatctacatctgg | |
| | gcgcccttggccgggacttgtggggtccttctcctgtcactggttatcaccc | |
| | tttactgccggagggaccagaggctgccccccgatgcccacaagccccctgg | |
| | gggaggcagtttccggacccccatccaagaggagcaggccgacgcccactcc | |
| | accctggccaagatcagagtgaagttcagcaggagcgcagacgcccccgcgt | |
| | accagcagggccagaaccagctctataacgagctcaatctaggacgaagaga | |
| | ggagtacgatgttttggacaagagacgtggccgggaccctgagatgggggga | |
| | aagccgagaaggaagaaccctcaggaaggcctgtacaatgaactgcagaaag | |
| | ataagatggcggaggcctacagtgagattgggatgaaaggcgagcgccggag | |
| | gggcaaggggcacgatggcctttaccagggtctcagtacagccaccaaggac | |
| | acctacgacgcccttcacatgcaggccctgccccctcgcggctctggcgagg | |
| | gaaggggttccctgcttacttgcggcgacgtcgaagagaatcccggtccgat | |
| | ggccctcccagtaactgccctccttttgcccctcgcactccttcttcatgcc | |
| | gctcgccccaactgggtcaacgtgattagcgatttgaagaaaatcgaggacc | |
| | ttatacagtctatgcatattgacgctacactgtatactgagagtgatgtaca | |
| | cccgtcctgtaaggtaacggccatgaaatgctttcttctggagctccaggtc | |
| | atcagcttggagtctggggacgcaagcatccacgatacggttgaaaacctca | |
| | tcatccttgcgaacaactctctctcatctaatggaaacgttacagagagtgg | |
| | gtgtaaggagtgcgaagagttggaagaaaaaaacatcaaagaatttcttcaa | |
| | tccttcgttcacatagtgcaaatgttcattaacacgtccactaccacacccg | |
| | ccccgaggccacctacgccggcaccgactatcgccagtcaacccctctctct | |
| | gcgccccgaggcttgccggcctgcggctggtggggcggtccacacccggggc | |
| | ctggattttgcgtgcgatatatacatctgggcacctcttgccggcacctgcg | |
| | gagtgctgcttctctcactcgttattacgctgtactgctaagcggccgcgtc | |
| | gac | |
| 2 | MALPVTALLLPLALLLHAARPDIQMTQSPSSLSASVGDRVTITCRASQDISK | CD19 CAR (aa) |
| | YLNWYQQKPGGTVKLLIYHTSRLHSGVPSRFSGSGSGTDFTLTISSLQPEDI | |
| | ATYFCQQGNTLPYTFGGGTKLEITGGGGSGGGGSGGGGSQVQLQESGPGLVK | |
| | PSQTLSLTCTVSGVSLPDYGVSWIRQPPGKGLEWIGVIWGSETTYYNSALKS | |
| | RLTISKDNSKNQVSLKLSSVTAADTAVYYCAKHYYYGGSYAMDYWGQGTSVT | |
| | VSSTTTPAPRPPTPAPTIASQPLSLRPEACRPAAGGAVHTRGLDFACDIYIW | |
| | APLAGTCGVLLLSLVITLYCRRDQRLPPDAHKPPGGGSFRTPIQEEQADAHS | |
| | TLAKIRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTATKD | |
| | TYDALHMQALPPRGSGEGRGSLLTCGDVEENPGPMALPVTALLLPLALLLHA | |
| | ARPNWVNVISDLKKIEDLIQSMHIDATLYTESDVHPSCKVTAMKCFLLELQV | |
| | ISLESGDASIHDTVENLIILANNSLSSNGNVTESGCKECEELEEKNIKEFLO | |
| | SFVHIVQMFINTSTTTPAPRPPTPAPTIASQPLSLRPEACRPAAGGAVHTRG | |
| | LDFACDIYIWAPLAGTCGVLLLSLVITLYC | |
| 3 | atggccttaccagtgaccgccttgctcctgccgctggccttgctgctccacg | CD19 CAR (nt) |
| | ccgccaggccggatattcagatgacccagagcccgagcagcctgagcgcgag | |
| | cgtgggcgatcgcgtgaccattacctgccgcgcgagccaggatattagcaaa | |
| | tatctgaactggtatcagcagaaaccgggcggcaccgtgaaactgctgattt | |
| | atcataccagccgcctgcatagcggcgtgccgagccgctttagcggcagcgg | |
| | cagcggcaccgattttaccctgaccattagcagcctgcagccggaagatatt | |
| | gcgacctatttttgccagcagggcaacaccctgccgtatacctttggcggcg | |
| | gcaccaaactggaaattaccggtggcggtggctcgggcggtggtgggtcggg | |
| | tggcggcggatctcaggtgcagctgcaggaaagcggcccgggcctggtgaaa | |
| | ccgagccagaccctgagcctgacctgcaccgtgagcggcgtgagcctgccgg | |
| | attatggcgtgagctggattcgccagccgccgggcaaaggcctggaatggat | |
| | tggcgtgatttggggcagcgaaaccacctattataacagcgcgctgaaaagc | |
| | cgcctgaccattagcaaagataacagcaaaaaccaggtgagcctgaaactga | |
| | gcagcgtgaccgcggcggataccgcggtgtattattgcgcgaaacattatta | |
| | ttatggcggcagctatgcgatggattattggggccagggcaccagcgtgacc | |
| | gtgagcagcaccacgacgccagcgccgcgaccaccaacaccggcgcccacca | |
| | tcgcgtcgcagcccctgtccctgcgcccagaggcgtgccggccagcggcggg | |
| | gggcgcagtgcacacgagggggctggacttcgcctgtgatatctacatctgg | |
| | gcgcccttggccgggacttgtggggtccttctcctgtcactggttatcaccc | |
| | tttactgccggagggaccagaggctgccccccgatgcccacaagccccctgg | |
| | gggaggcagtttccggacccccatccaagaggagcaggccgacgcccactcc | |
| | accctggccaagatcagagtgaagttcagcaggagcgcagacgcccccgcgt | |
| | accagcagggccagaaccagctctataacgagctcaatctaggacgaagaga | |
| | ggagtacgatgttttggacaagagacgtggccgggaccctgagatgggggga | |
| | aagccgagaaggaagaaccctcaggaaggcctgtacaatgaactgcagaaag | |
| | ataagatggcggaggcctacagtgagattgggatgaaaggcgagcgccggag | |
| | gggcaaggggcacgatggcctttaccagggtctcagtacagccaccaaggac | |
| | acctacgacgcccttcacatgcaggccctgccccctcgc | |
| 4 | MALPVTALLLPLALLLHAARPDIQMTQSPSSLSASVGDRVTITCRASQDISK | CD19 CAR (aa) |
| | YLNWYQQKPGGTVKLLIYHTSRLHSGVPSRFSGSGSGTDFTLTISSLQPEDI | |
| | ATYFCQQGNTLPYTFGGGTKLEITGGGGSGGGGSGGGGSQVQLQESGPGLVK | |
| | PSQTLSLTCTVSGVSLPDYGVSWIRQPPGKGLEWIGVIWGSETTYYNSALKS | |
| | RLTISKDNSKNQVSLKLSSVTAADTAVYYCAKHYYYGGSYAMDYWGQGTSVT | |
| | VSSTTTPAPRPPTPAPTIASQPLSLRPEACRPAAGGAVHTRGLDFACDIYIW | |
| | APLAGTCGVLLLSLVITLYCRRDQRLPPDAHKPPGGGSFRTPIQEEQADAHS | |
| | TLAKIRVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTATKD | |
| | TYDALHMQALPPR | |
| 5 | gatattcagatgacccagagcccgagcagcctgagcgcgagcgtgggcgatc | CD19 scFv (nt) |
| | gcgtgaccattacctgccgcgcgagccaggatattagcaaatatctgaactg | |
| | gtatcagcagaaaccgggcggcaccgtgaaactgctgatttatcataccagc | |
| | cgcctgcatagcggcgtgccgagccgctttagcggcagcggcagcggcaccg | |
| | attttaccctgaccattagcagcctgcagccggaagatattgcgacctattt | |
| | ttgccagcagggcaacaccctgccgtatacctttggcggcggcaccaaactg | |
| | gaaattaccggtggcggtggctcgggcggtggtgggtcgggtggcggcggat | |
| | ctcaggtgcagctgcaggaaagcggcccgggcctggtgaaaccgagccagac | |
| | cctgagcctgacctgcaccgtgagcggcgtgagcctgccggattatggcgtg | |
| | agctggattcgccagccgccgggcaaaggcctggaatggattggcgtgattt | |
| | ggggcagcgaaaccacctattataacagcgcgctgaaaagccgcctgaccat | |
| | tagcaaagataacagcaaaaaccaggtgagcctgaaactgagcagcgtgacc | |
| | gcggcggataccgcggtgtattattgcgcgaaacattattattatggcggca | |
| | gctatgcgatggattattggggccagggcaccagcgtgaccgtgagcagc | |
| 6 | DIQMTQSPSSLSASVGDRVTITCRASQDISKYLNWYQQKPGGTVKLLIYHTS | CD19 scFv [aa) |
| | RLHSGVPSRFSGSGSGTDFTLTISSLQPEDIATYFCQQGNTLPYTFGGGTKL | |
| | EITGGGGGGGGSGGGGSQVQLQESGPGLVKPSQTLSLTCTVSGVSLPDYGVS | |
| | WIRQPPGKGLEWIGVIWGSETTYYNSALKSRLTISKDNSKNQVSLKLSSVTA | |
| | ADTAVYYCAKHYYYGGSYAMDYWGQGTSVTVSS | |
| 7 | atggccttaccagtgaccgccttgctcctgccgctggccttgctgctccacg | CD8 signal peptide |
| | ccgccaggccg | (nt) |
| 8 | MALPVTALLLPLALLLHAARP | CD8 signal peptide |
| | | (aa) |
| 9 | gatattcagatgacccagagcccgagcagcctgagcgcgagcgtgggcgatc | CD19 VL (nt) |
| | gcgtgaccattacctgccgcgcgagccaggatattagcaaatatctgaactg | |
| | gtatcagcagaaaccgggcggcaccgtgaaactgctgatttatcataccagc | |
| | cgcctgcatagcggcgtgccgagccgctttagcggcagcggcagcggcaccg | |
| | attttaccctgaccattagcagcctgcagccggaagatattgcgacctattt | |
| | ttgccagcagggcaacaccctgccgtatacctttggcggcggcaccaaactg | |
| | gaaattacc | |
| 10 | cgcgcgagccaggatattagcaaatatctgaac | CDR-L1 (nt) |
| 11 | cataccagccgcctgcatagc | CDR-L2 (nt) |
| 12 | cagcagggcaacaccctgccgtatacc | CDR-L3 (nt) |
| 13 | DIQMTQSPSSLSASVGDRVTITCRASQDISKYLNWYQQKPGGTVKLLIYHTSR | CD19 VL (aa) |
| | LHSGVPSRFSGSGSGTDFTLTISSLQPEDIATYFCQQGNTLPYTFGGGTKLEI | |
| | T | |
| 14 | RASQDISKYLN | CDR-L1 (aa) |
| 15 | HTSRLHS | CDR-L2 (aa) |
| 16 | QQGNTLPYT | CDR-L3 (aa) |
| 17 | caggtgcagctgcaggaaagcggcccgggcctggtgaaaccgagccagaccc | CD19 VH (nt) |
| | tgagcctgacctgcaccgtgagcggcgtgagcctgccggattatggcgtgag | |
| | ctggattcgccagccgccgggcaaaggcctggaatggattggcgtgatttgg | |
| | ggcagcgaaaccacctattataacagcgcgctgaaaagccgcctgaccatta | |
| | gcaaagataacagcaaaaaccaggtgagcctgaaactgagcagcgtgaccgc | |
| | ggcggataccgcggtgtattattgcgcgaaacattattattatggcggcagc | |
| | tatgcgatggattattggggccagggcaccagcgtgaccgtgagcagc | |
| 18 | ggcgtgagcctgccggattatggcgtgagc | CDR-H1 (nt) |
| 19 | gtgatttggggcagcgaaaccacctattataacagcgcgctgaaaagc | CDR-H2 (nt) |
| 20 | cattattattatggcggcagctatgcgatggattat | CDR-H3 (nt) |
| 21 | QVQLQESGPGLVKPSQTLSLTCTVSGVSLPDYGVSWIRQPPGKGLEWIGVIWG | CD19 VH (aa) |
| | SETTYYNSALKSRLTISKDNSKNQVSLKLSSVTAADTAVYYCAKHYYYGGSYA | |
| | MDYWGQGTSVTVSS | |
| 22 | GVSLPDYGVS | CDR-H1 (aa) |
| 23 | VIWGSETTYYNSALKS | CDR-H2 (aa) |
| 24 | HYYYGGSYAMDY | CDR-H3 (a) |
| 25 | ggtggcggtggctcgggcggtggtgggtcgggtggcggcggatct | Linker (nt) |
| 26 | GGGGSGGGGSGGGGS | Linker (aa) |
| 27 | accacgacgccagcgccgcgaccaccaacaccggcgcccaccatcgcgtcgc | CD8 hinge (nt) |
| | agcccctgtccctgcgcccagaggcgtgccggccagcggcggggggcgcagt | |
| | gcacacgagggggctggacttcgcctgtgat | |
| 28 | TTTPAPRPPTPAPTIASQPLSLRPEACRPAAGGAVHTRGLDFACD | CD8 hinge (aa) |
| 29 | atatacatctgggcacctcttgccggcacctgcggagtgctgcttctctcac | CD8 TM region (nt) |
| | tcgttattacgctgtactgctaagc | |
| 30 | IYIWAPLAGTCGVLLLSLVITLYC | CD8 TM region (aa) |
| 31 | cggagggaccagaggctgccccccgatgcccacaagccccctgggggaggca | OX40 (nt) |
| | gtttccggacccccatccaagaggagcaggccgacgcccactccaccctggc | |
| | caagatc | |
| 32 | RRDQRLPPDAHKPPGGGSFRTPIQEEQADAHSTLAKI | OX40 (aa) |
| 33 | agagtgaagttcagcaggagcgcagacgcccccgcgtaccagcagggccaga | CD3 zeta (nt) |
| | accagctctataacgagctcaatctaggacgaagagaggagtacgatgtttt | |
| | ggacaagagacgtggccgggaccctgagatggggggaaagccgagaaggaag | |
| | aaccctcaggaaggcctgtacaatgaactgcagaaagataagatggcggagg | |
| | cctacagtgagattgggatgaaaggcgagcgccggaggggcaaggggcacga | |
| | tggcctttaccagggtctcagtacagccaccaaggacacctacgacgccctt | |
| | cacatgcaggccctgccccctcgc | |
| 34 | RVKFSRSADAPAYQQGQNQLYNELNLGRREEYDVLDKRRGRDPEMGGKPRRK | CD3 zeta (aa) |
| | NPQEGLYNELQKDKMAEAYSEIGMKGERRRGKGHDGLYQGLSTATKDTYDAL | |
| | HMQALPPR | |
| 35 | ggctctggcgagggaaggggttccctgcttacttgcggcgacgtcgaagaga | T2A (nt) |
| | atcccggtccg | |
| 36 | GSGEGRGSLLTCGDVEENPGP | T2A (aa) |
| 37 | aactgggtcaacgtgattagcgatttgaagaaaatcgaggaccttatacagt | IL15 (nt) |
| | ctatgcatattgacgctacactgtatactgagagtgatgtacacccgtcctg | |
| | taaggtaacggccatgaaatgctttcttctggagctccaggtcatcagcttg | |
| | gagtctggggacgcaagcatccacgatacggttgaaaacctcatcatccttg | |
| | cgaacaactctctctcatctaatggaaacgttacagagagtgggtgtaagga | |
| | gtgcgaagagttggaagaaaaaaacatcaaagaatttcttcaatccttcgtt | |
| | cacatagtgcaaatgttcattaacacgtcc | |
| 38 | NWVNVISDLKKIEDLIQSMHIDATLYTESDVHPSCKVTAMKCFLLELQVISL | IL15 (aa) |
| | ESGDASIHDTVENLIILANNSLSSNGNVTESGCKECEELEEKNIKEFLQSFV | |
| | HIVQMFINTS | |
| 39 | atggccctcccagtaactgccctccttttgcccctcgcactccttcttcatg | mbIL15 (nt) |
| | ccgctcgccccaactgggtcaacgtgattagcgatttgaagaaaatcgagga | |
| | ccttatacagtctatgcatattgacgctacactgtatactgagagtgatgta | |
| | cacccgtcctgtaaggtaacggccatgaaatgctttcttctggagctccagg | |
| | tcatcagcttggagtctggggacgcaagcatccacgatacggttgaaaacct | |
| | catcatccttgcgaacaactctctctcatctaatggaaacgttacagagagt | |
| | gggtgtaaggagtgcgaagagttggaagaaaaaaacatcaaagaatttcttc | |
| | aatccttcgttcacatagtgcaaatgttcattaacacgtccactaccacacc | |
| | cgccccgaggccacctacgccggcaccgactatcgccagtcaacccctctct | |
| | ctgcgccccgaggcttgccggcctgcggctggtggggcggtccacacccggg | |
| | gcctggattttgcgtgcgatatatacatctgggcacctcttgccggcacctg | |
| | cggagtgctgcttctctcactcgttattacgctgtactgctaagc | |
| 40 | MALPVTALLLPLALLLHAARPNWVNVISDLKKIEDLIQSMHIDATLYTESDV | mbIL15 (aa) |
| | HPSCKVTAMKCFLLELQVISLESGDASIHDTVENLIILANNSLSSNGNVTES | |
| | GCKECEELEEKNIKEFLQSFVHIVQMFINTSTTTPAPRPPTPAPTIASQPLS | |
| | LRPEACRPAAGGAVHTRGLDFACDIYIWAPLAGTCGVLLLSLVITLYC | |
| 41 | atggccttaccagtgaccgccttgctcctgccgctggccttgctgctccacg | CD19 CAR (nt) |
| | ccgccaggccggatattcagatgacccagagcccgagcagcctgagcgcgag | |
| | cgtgggcgatcgcgtgaccattacctgccgcgcgagccaggatattagcaaa | |
| | tatctgaactggtatcagcagaaaccgggcggcaccgtgaaactgctgattt | |
| | atcataccagccgcctgcatagcggcgtgccgagccgctttagcggcagcgg | |
| | cagcggcaccgattttaccctgaccattagcagcctgcagccggaagatatt | |
| | gcgacctatttttgccagcagggcaacaccctgccgtatacctttggcggcg | |
| | gcaccaaactggaaattaccggtggcggtggctcgggcggtggtgggtcggg | |
| | tggcggcggatctcaggtgcagctgcaggaaagcggcccgggcctggtgaaa | |
| | ccgagccagaccctgagcctgacctgcaccgtgagcggcgtgagcctgccgg | |
| | attatggcgtgagctggattcgccagccgccgggcaaaggcctggaatggat | |
| | tggcgtgatttggggcagcgaaaccacctattataacagcgcgctgaaaagc | |
| | cgcctgaccattagcaaagataacagcaaaaaccaggtgagcctgaaactga | |
| | gcagcgtgaccgcggcggataccgcggtgtattattgcgcgaaacattatta | |
| | ttatggcggcagctatgcgatggattattggggccagggcaccagcgtgacc | |
| | gtgagcagcaccacgacgccagcgccgcgaccaccaacaccggcgcccacca | |
| | tcgcgtcgcagcccctgtccctgcgcccagaggcgtgccggccagcggcggg | |
| | gggcgcagtgcacacgagggggctggacttcgcctgtgatatctacatctgg | |
| | gcgcccttggccgggacttgtggggtccttctcctgtcactggttatcaccc | |
| | tttactgccggagggaccagaggctgccccccgatgcccacaagccccctgg | |
| | gggaggcagtttccggacccccatccaagaggagcaggccgacgcccactcc | |
| | accctggccaagatcagagtgaagttcagcaggagcgcagacgcccccgcgt | |
| | accagcagggccagaaccagctctataacgagctcaatctaggacgaagaga | |
| | ggagtacgatgttttggacaagagacgtggccgggaccctgagatgggggga | |
| | aagccgagaaggaagaaccctcaggaaggcctgtacaatgaactgcagaaag | |
| | ataagatggcggaggcctacagtgagattgggatgaaaggcgagcgccggag | |
| | gggcaaggggcacgatggcctttaccagggtctcagtacagccaccaaggac | |
| | acctacgacgcccttcacatgcaggccctgccccctcgcggctctggcgagg | |
| | gaaggggttccctgcttacttgcggcgacgtcgaagagaatcccggtccgat | |
| | ggccctcccagtaactgccctccttttgcccctcgcactccttcttcatgcc | |
| | gctcgccccaactgggtcaacgtgattagcgatttgaagaaaatcgaggacc | |
| | ttatacagtctatgcatattgacgctacactgtatactgagagtgatgtaca | |
| | cccgtcctgtaaggtaacggccatgaaatgctttcttctggagctccaggtc | |
| | atcagcttggagtctggggacgcaagcatccacgatacggttgaaaacctca | |
| | tcatccttgcgaacaactctctctcatctaatggaaacgttacagagagtgg | |
| | gtgtaaggagtgcgaagagttggaagaaaaaaacatcaaagaatttcttcaa | |
| | tccttcgttcacatagtgcaaatgttcattaacacgtccactaccacacccg | |
| | ccccgaggccacctacgccggcaccgactatcgccagtcaacccctctctct | |
| | gcgccccgaggcttgccggcctgcggctggtggggcggtccacacccggggc | |
| | ctggattttgcgtgcgatatatacatctgggcacctcttgccggcacctgcg | |
| | gagtgctgcttctctcactcgttattacgctgtactgctaagc | |
| 42 | DIQMTQSPSSLSASVGDRVTITCRASQDISKYLNWYQQKPGGTVKLLIYHTS | CD19 CAR (aa) |
| | RLHSGVPSRFSGSGSGTDFTLTISSLQPEDIATYFCQQGNTLPYTFGGGTKL | |
| | EITGGGGSGGGGSGGGGSQVQLQESGPGLVKPSQTLSLTCTVSGVSLPDYGV | |
| | SWIRQPPGKGLEWIGVIWGSETTYYNSALKSRLTISKDNSKNQVSLKLSSVT | |
| | AADTAVYYCAKHYYYGGSYAMDYWGQGTSVTVSSTTTPAPRPPTPAPTIASQ | |
| | PLSLRPEACRPAAGGAVHTRGLDFACDIYIWAPLAGTCGVLLLSLVITLYCR | |
| | RDQRLPPDAHKPPGGGSFRTPIQEEQADAHSTLAKIRVKFSRSADAPAYQQG | |
| | QNQLYNELNLGRREEYDVLDKRRGRDPEMGGKPRRKNPQEGLYNELQKDKMA | |
| | EAYSEIGMKGERRRGKGHDGLYQGLSTATKDTYDALHMQALPPRGSGEGRGS | |
| | LLTCGDVEENPGPMALPVTALLLPLALLLHAARPNWVNVISDLKKIEDLIQS | |
| | MHIDATLYTESDVHPSCKVTAMKCFLLELQVISLESGDASIHDTVENLIILA | |
| | NNSLSSNGNVTESGCKECEELEEKNIKEFLQSFVHIVQMFINTSTTTPAPRP | |
| | PTPAPTIASQPLSLRPEACRPAAGGAVHTRGLDFACDIYIWAPLAGTCGVLL | |
| | LSLVITLYC | |
| 43 | DIQMTQSPSSLSASVGDRVTITCRASQDISKYLNWYQQKPGGTVKLLIYHTS | CD19 CAR (aa) |
| | RLHSGVPSRFSGSGSGTDFTLTISSLQPEDIATYFCQQGNTLPYTFGGGTKL | |
| | EITGGGGSGGGGSGGGGSQVQLQESGPGLVKPSQTLSLTCTVSGVSLPDYGV | |
| | SWIRQPPGKGLEWIGVIWGSETTYYNSALKSRLTISKDNSKNQVSLKLSSVT | |
| | AADTAVYYCAKHYYYGGSYAMDYWGQGTSVTVSSTTTPAPRPPTPAPTIASQ | |
| | PLSLRPEACRPAAGGAVHTRGLDFACDIYIWAPLAGTCGVLLLSLVITLYCR | |
| | RDQRLPPDAHKPPGGGSFRTPIQEEQADAHSTLAKIRVKFSRSADAPAYQQG | |
| | QNQLYNELNLGRREEYDVLDKRRGRDPEMGGKPRRKNPQEGLYNELQKDKMA | |
| | EAYSEIGMKGERRRGKGHDGLYQGLSTATKDTYDALHMQALPPR | |
| 44 | NWVNVISDLKKIEDLIQSMHIDATLYTESDVHPSCKVTAMKCFLLELQVIS | mbIL15 (aa) |
| | LESGDASIHDTVENLIILANNSLSSNGNVTESGCKECEELEEKNIKEFLQS | |
| | FVHIVQMFINTSTTTPAPRPPTPAPTIASQPLSLRPEACRPAAGGAVHTRG | |
| | LDFACDIYIWAPLAGTCGVLLLSLVITLYC | |
| 45 | GVSLPDYG | CDR-H1 (aa) |
| 46 | IWGSETT | CDR-H2 (aa) |
| 47 | AKHYYYGGSYAMDY | CDR-H3 (aa) |
| 48 | QDISKY | CRD-L1 (aa) |
| 49 | HT | CDR-L2 (aa) |
| 50 | DYGVS | CDR-H1 (aa) |
| 51 | TTTPAPRPPTPAPTIASQPLSLRPEACRPAAGGAVHTRGLDFACDIYIWAPL | CD8 TM domain (aa) |
| | AGTCGVLLLSLVIT | |
| 52 | TTTPAPRPPTPAPTIASQPLSLRPEACRPAAGGAVHTRGLDFACDIYIWAPL | CD8 TM domain (aa) |
| | AGTCGVLLLSLVITLYC | |
| 53 | atggccttaccagtgaccgccttgctcctgccgctggccttgctgctccacg | CD19 CAR (nt) |
| | ccgccaggccggatattcagatgacccagagcccgagcagcctgagcgcgag | |
| | cgtgggcgatcgcgtgaccattacctgccgcgcgagccaggatattagcaaa | |
| | tatctgaactggtatcagcagaaaccgggcggcaccgtgaaactgctgattt | |
| | atcataccagccgcctgcatagcggcgtgccgagccgctttagcggcagcgg | |
| | cagcggcaccgattttaccctgaccattagcagcctgcagccggaagatatt | |
| | gcgacctatttttgccagcagggcaacaccctgccgtatacctttggcggcg | |
| | gcaccaaactggaaattaccggtggcggtggctcgggcggtggtgggtcggg | |
| | tggcggcggatctcaggtgcagctgcaggaaagcggcccgggcctggtgaaa | |
| | ccgagccagaccctgagcctgacctgcaccgtgagcggcgtgagcctgccgg | |
| | attatggcgtgagctggattcgccagccgccgggcaaaggcctggaatggat | |
| | tggcgtgatttggggcagcgaaaccacctattataacagcgcgctgaaaagc | |
| | cgcctgaccattagcaaagataacagcaaaaaccaggtgagcctgaaactga | |
| | gcagcgtgaccgcggcggataccgcggtgtattattgcgcgaaacattatta | |
| | ttatggcggcagctatgcgatggattattggggccagggcaccagcgtgacc | |
| | gtgagcagcaccacgacgccagcgccgcgaccaccaacaccggcgcccacca | |
| | tcgcgtcgcagcccctgtccctgcgcccagaggcgtgccggccagcggcggg | |
| | gggcgcagtgcacacgagggggctggacttcgcctgtgatatctacatctgg | |
| | gcgcccttggccgggacttgtggggtccttctcctgtcactggttatcaccc | |
| | tttactgccggagggaccagaggctgccccccgatgcccacaagccccctgg | |
| | gggaggcagtttccggacccccatccaagaggagcaggccgacgcccactcc | |
| | accctggccaagatcagagtgaagttcagcaggagcgcagacgcccccgcgt | |
| | accagcagggccagaaccagctctataacgagctcaatctaggacgaagaga | |
| | ggagtacgatgttttggacaagagacgtggccgggaccctgagatgggggga | |
| | aagccgagaaggaagaaccctcaggaaggcctgtacaatgaactgcagaaag | |
| | ataagatggcggaggcctacagtgagattgggatgaaaggcgagcgccggag | |
| | gggcaaggggcacgatggcctttaccagggtctcagtacagccaccaaggac | |
| | acctacgacgcccttcacatgcaggccctgccccctcgcggctctggcgagg | |
| | gaaggggttccctgcttacttgcggcgacgtcgaagagaatcccggtccgat | |
| | ggccctcccagtaactgccctccttttgcccctcgcactccttcttcatgcc | |
| | gctcgccccaactgggtcaacgtgattagcgatttgaagaaaatcgaggacc | |
| | ttatacagtctatgcatattgacgctacactgtatactgagagtgatgtaca | |
| | cccgtcctgtaaggtaacggccatgaaatgctttcttctggagctccaggtc | |
| | atcagcttggagtctggggacgcaagcatccacgatacggttgaaaacctca | |
| | tcatccttgcgaacaactctctctcatctaatggaaacgttacagagagtgg | |
| | gtgtaaggagtgcgaagagttggaagaaaaaaacatcaaagaatttcttcaa | |
| | tccttcgttcacatagtgcaaatgttcattaacacgtccactaccacacccg | |
| | ccccgaggccacctacgccggcaccgactatcgccagtcaacccctctctct | |
| | gcgccccgaggcttgccggcctgcggctggtggggcggtccacacccggggc | |
| | ctggattttgcgtgcgatatatacatctgggcacctcttgccggcacctgcg | |
| | gagtgctgcttctctcactcgttattacgctgtactgc | |

In several embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, codon optimization, or other types of modifications.

In accordance with some embodiments described herein, any of the sequences may be used, or a truncated or mutated form of any of the sequences disclosed herein (and/or included in the accompanying sequence listing) may be used and in any combination.

A Sequence Listing in electronic format may be submitted herewith. Some of the sequences provided in the Sequence Listing may be designated as Artificial Sequences by virtue of being non-naturally occurring fragments or portions of other sequences, including naturally occurring sequences. Some of the sequences provided in the Sequence Listing may be designated as Artificial Sequences by virtue of being combinations of sequences from different origins, such as humanized antibody sequences. Appendix A is attached and incorporated by reference herein and provides sequence information for nucleic acid and amino acid sequences provided for herein.

1. A method of treatment comprising administering genetically engineered natural killer (NK) cells to a subject having a disease or condition associated with CD19 expression, wherein the method comprises
administering at least a first dosing cycle to the subject, wherein the first dosing cycle comprises a first dose of genetically engineered natural killer (NK) cells administered to the subject at a first time point, a second dose of genetically engineered NK cells administered to the subject between 5-10 days after the first time point, and a third dose of genetically engineered NK cells administered to the subject between 5-10 days after the second dose, wherein:
each of the first, second and third doses comprise at least about 1×10⁹ NK cells, at least a portion of the genetically engineered NK cells is engineered to express a chimeric antigen receptor (CAR) that binds to CD19, and
first dosing cycle is initiated after the subject has undergone a lymphodepletion process that reduces native immune cell numbers.

2. The method of claim 1, wherein the first dosing cycle is followed by one or more additional dosing cycle.

3. The method of claim 1, wherein, if the subject exhibits a clinical response, following the first dosing cycle, the dosing regimen comprises an additional dosing cycle.

4-6. (canceled)

7. The method of claim 1, wherein each dosing cycle is between about 14 days and about 35 days.

8-15. (canceled)

16. The method of claim 1, further comprising administering a therapeutic agent that targets CD20 to the subject.

17. (canceled)

18. The method of claim 16, wherein the therapeutic agent is an anti-CD20 monoclonal antibody.

19-26. (canceled)

27. The method of claim 1, wherein the disease or condition is a cancer.

28. The method of claim 1, wherein the disease or condition is a B cell cancer.

29-44. (canceled)

45. The method of claim 1, wherein the CAR comprises:
(a) an extracellular antigen-binding moiety that binds to CD19;
(b) a transmembrane domain; and
(c) an intracellular signaling domain comprising an OX40 domain and a CD3zeta domain.

46. The method of claim 45, wherein the antigen-binding moiety comprises a heavy chain variable region (VH) and a light chain variable region (VL), wherein
the VH comprises a CDR-1, a CDR-2, and a CDR-3 comprising the amino acid sequences set forth in SEQ ID NOS: 45, 46, and 47, respectively; and the VL comprises a CDR-1, a CDR-2, and a CDR-3 comprising the amino acid sequences set forth in SEQ ID NOS: 48, 49, and 16, respectively.

47-49. (canceled)

50. The method of claim 1, wherein the genetically engineered NK cells are allogeneic with respect to the subject.

51-70. (canceled)

71. A method of treatment comprising administering genetically engineered natural killer (NK) cells expressing a chimeric antigen receptor (CAR) that binds to CD19 to a subject having a cancer, wherein the cancer does not express CD58 or expresses a mutated form of CD58.

72. The method of claim 71, further comprising:
determining that the cancer that does not express CD58 or expresses a mutated form of CD58; or
identifying the subject as having a cancer that does not express CD58 or expresses a mutated form of CD58.

73. The method of claim 71, wherein the subject was previously treated with genetically engineered T cells expressing a CAR that binds to CD19.

74. The method of claim 73, wherein the subject is relapsed and/or refractory to the genetically engineered T cells.

75-86. (canceled)

87. The method of claim 71, wherein the cancer is a B cell cancer.

88-93. (canceled)

94. The method of claim 71, wherein the CAR comprises:
(a) an extracellular antigen-binding moiety that binds to CD19;
(b) a transmembrane domain; and
(c) an intracellular signaling domain comprising an OX40 domain and a CD3zeta domain.

95. The method of claim 94, wherein the antigen-binding moiety comprises a heavy chain variable region (VH) and a light chain variable region (VL), wherein
the VH comprises a CDR-1, a CDR-2, and a CDR-3 comprising the amino acid sequences set forth in SEQ ID NOS: 45, 46, and 47, respectively; and the VL comprises a CDR-1, a CDR-2, and a CDR-3 comprising the amino acid sequences set forth in SEQ ID NOS: 48, 49, and 16, respectively.

96-106. (canceled)

107. The method of claim 46, wherein the genetically engineered NK cells are allogeneic with respect to the subject.

108. The method of claim 71, wherein the genetically engineered NK cells are allogeneic with respect to the subject.


SEQUENCE LISTING (53 sequences)

SEQ ID NO: 1 - DNA, 2135 bp, Synthetic construct
  source 1..2135: mol_type = other DNA
atggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 2 - protein, 706 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 3 - DNA, 1443 bp, Synthetic construct
  source 1..1443: mol_type = other DNA
atggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 4 - protein, 481 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 726 bp, Synthetic construct
  source 1..726: mol_type = other DNA
gatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagc

SEQ ID NO: 6 - protein, 242 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSS

SEQ ID NO: 7 - DNA, 63 bp, Synthetic construct
  source 1..63: mol_type = other DNA
atggccttaccagtgaccgccttgctcctgccgctggccttgctgctccacgccgccaggccg

SEQ ID NO: 8 - protein, 21 aa, Synthetic construct
MALPVTALLLPLALLLHAARP

SEQ ID NO: 9 - DNA, 321 bp, Synthetic construct
  source 1..321: mol_type = other DNA
gatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattacc

SEQ ID NO: 10 - DNA, 33 bp, Synthetic construct
  source 1..33: mol_type = other DNA
cgcgcgagccaggatattagcaaatatctgaac

SEQ ID NO: 11 - DNA, 21 bp, Synthetic construct
  source 1..21: mol_type = other DNA
cataccagccgcctgcatagc

SEQ ID NO: 12 - DNA, 27 bp, Synthetic construct
  source 1..27: mol_type = other DNA
cagcagggcaacaccctgccgtatacc

SEQ ID NO: 13 - protein, 107 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEIT

SEQ ID NO: 14 - protein, 11 aa, Synthetic construct
RASQDISKYLN

SEQ ID NO: 15 - protein, 7 aa, Synthetic construct
HTSRLHS

SEQ ID NO: 16 - protein, 9 aa, Synthetic construct
QQGNTLPYT

SEQ ID NO: 17 - DNA, 360 bp, Synthetic construct
  source 1..360: mol_type = other DNA
caggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagc

SEQ ID NO: 18 - DNA, 30 bp, Synthetic construct
  source 1..30: mol_type = other DNA
ggcgtgagcctgccggattatggcgtgagc

SEQ ID NO: 19 - DNA, 48 bp, Synthetic construct
  source 1..48: mol_type = other DNA
gtgatttggggcagcgaaaccacctattataacagcgcgctgaaaagc

SEQ ID NO: 20 - DNA, 36 bp, Synthetic construct
  source 1..36: mol_type = other DNA
cattattattatggcggcagctatgcgatggattat

SEQ ID NO: 21 - protein, 120 aa, Synthetic construct
QVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSS

SEQ ID NO: 22 - protein, 10 aa, Synthetic construct
GVSLPDYGVS

SEQ ID NO: 23 - protein, 16 aa, Synthetic construct
VIWGSETTYYNSALKS

SEQ ID NO: 24 - protein, 12 aa, Synthetic construct
HYYYGGSYAMDY

SEQ ID NO: 25 - DNA, 45 bp, Synthetic construct
  source 1..45: mol_type = other DNA
ggtggcggtggctcgggcggtggtgggtcgggtggcggcggatct

SEQ ID NO: 26 - protein, 15 aa, Synthetic construct
GGGGSGGGGSGGGGS

SEQ ID NO: 27 - DNA, 135 bp, Synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 28 - protein, 45 aa, Synthetic construct
TTTPAPRPPTPAPTIASQPLSLRPEACRPAAGGAVHTRGLDFACD

SEQ ID NO: 29 - DNA, 77 bp, Synthetic construct
  source 1..77: mol_type = other DNA
atatacatctgggcacctcttgccggcacctgcggagtgctgcttctctcactcgttattacgctgtactgctaagc

SEQ ID NO: 30 - protein, 24 aa, Synthetic construct
IYIWAPLAGTCGVLLLSLVITLYC

SEQ ID NO: 31 - DNA, 111 bp, Synthetic construct
  source 1..111: mol_type = other DNA
cggagggaccagaggctgccccccgatgcccacaagccccctgggggaggcagtttccggacccccatccaagaggagcaggccgacgcccactccaccctggccaagatc

SEQ ID NO: 32 - protein, 37 aa, Synthetic construct
RRDQRLPPDAHKPPGGGSFRTPIQEEQADAHSTLAKI

SEQ ID NO: 33 - DNA, 336 bp, Synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 34 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 63 bp, Synthetic construct
  source 1..63: mol_type = other DNA
ggctctggcgagggaaggggttccctgcttacttgcggcgacgtcgaagagaatcccggtccg

SEQ ID NO: 36 - protein, 21 aa, Synthetic construct
GSGEGRGSLLTCGDVEENPGP

SEQ ID NO: 37 - DNA, 342 bp, Homo sapiens
  source 1..342: mol_type = other DNA
aactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtcc

SEQ ID NO: 38 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 39 - DNA, 617 bp, Synthetic construct
  source 1..617: mol_type = other DNA
atggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagc

SEQ ID NO: 40 - protein, 204 aa, Synthetic construct
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 41 - DNA, 2123 bp, Synthetic construct
  source 1..2123: mol_type = other DNA
atggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagc

SEQ ID NO: 42 - protein, 685 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 43 - protein, 460 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 183 aa, Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 45 - protein, 8 aa, synthetic construct
GVSLPDYG

SEQ ID NO: 46 - protein, 7 aa, synthetic construct
IWGSETT

SEQ ID NO: 47 - protein, 14 aa, synthetic construct
AKHYYYGGSYAMDY

SEQ ID NO: 48 - protein, 6 aa, synthetic construct
QDISKY

SEQ ID NO: 49-50 - skipped (2 entries)

SEQ ID NO: 51 - protein, 66 aa, Homo sapiens
TTTPAPRPPTPAPTIASQPLSLRPEACRPAAGGAVHTRGLDFACDIYIWAPLAGTCGVLLLSLVIT

SEQ ID NO: 52 - protein, 69 aa, Homo sapiens
TTTPAPRPPTPAPTIASQPLSLRPEACRPAAGGAVHTRGLDFACDIYIWAPLAGTCGVLLLSLVITLYC

SEQ ID NO: 53 - DNA, 2118 bp, synthetic construct
  source 1..2118: mol_type = other DNA
atggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc